(19) United States
(12) Patent Application Publication
(10) Pub. No.: US 2026/0209747 A1
(43) Pub. Date: Jul. 23, 2026

(54) DIRECTED EVOLUTION OF ENGINEERED VIRUS-LIKE PARTICLES (EVLPS)

(71) Applicants: The Broad Institute, Inc., Cambridge, MA (US); President and Fellows of Harvard College, Cambridge, MA (US)

(72) Inventors: David R. Liu, Cambridge, MA (US); Aditya Raguram, Cambridge, MA (US)

(73) Assignees: The Broad Institute, Inc., Cambridge, MA (US); President and Fellows of Harvard College, Cambridge, MA (US)

(21) Appl. No.: 19/354,048

(22) Filed: Oct. 9, 2025

Related U.S. Application Data
(63) Continuation of application No. PCT/US2024/023695, filed on Apr. 9, 2024.
(60) Provisional application No. 63/458,310, filed on Apr. 10, 2023.
(60) Provisional application No. 63/578,190, filed on Aug. 23, 2023.

(51) Int. Cl. C12N 15/10; C07K 14/005; C12N 15/86

(52) U.S. Cl. CPC C12N 15/1058; C07K 14/005; C12N 15/1065; C12N 15/86; C12N 2740/13022; C12N 2740/15023; C12N 2740/15043

(57) ABSTRACT

The present disclosure provides methods, compositions, and systems for evolving virus-like particles (VLPs) having one or more desired properties such as increased production levels, increased cargo packaging efficiency, and/or increased transduction of particular target cell types of interest. The present disclosure also provides libraries for use in such methods, and methods for producing the libraries. Group specific antigen (gag) proteins comprising nucleocapsid protein variants evolved using the methods described herein are also provided herein. The present disclosure also provides VLPs comprising such gag proteins comprising nucleocapsid protein variants. Polynucleotides, vectors, cells, and kits useful for performing the methods described herein are also provided.

### RELATED APPLICATIONS

This application claims priority under 35 U.S.C. §§ 120 and 365(c) to International PCT Application, PCT/US2024/023695, filed Apr. 9, 2024, which claims priority under 35 U.S.C. § 119(e) to U.S. Provisional Application, U.S. Ser. No. 63/458,310, filed Apr. 10, 2023, and U.S. Provisional Application, U.S. Ser. No. 63/578,190, filed Aug. 23, 2023, each of which is incorporated herein by reference.

### GOVERNMENT SUPPORT

This invention was made with government support under Grant Nos. UG3AI150551, U01AI142756, R35GM118062, RM1HG009490, and R01EY009339, awarded by the National Institutes of Health. The government has certain rights in the invention.

### REFERENCE TO AN ELECTRONIC SEQUENCE LISTING

The contents of the electronic sequence listing (B119570182US02-SEQ-TNG.xml; Size: 103,363 bytes; and Date of Creation: Oct. 2, 2025) are herein incorporated by reference in their entirety.

### BACKGROUND OF THE INVENTION

Recently developed gene editing agents, such as base editors and prime editors, enable the precise manipulation of genomic DNA in living organisms and raise the possibility of treating the root cause of many genetic diseases. The broad therapeutic application of in vivo gene editing requires safe and efficient methods for delivering gene editing agents to multiple tissues and organs. Many of the most robust approaches for delivering gene editing agents in vivo reported to date involve the use of viruses, such as adeno-associated viruses (AAVs) or lentiviruses (LV), to deliver gene editing agent-encoding DNA to target tissues. However, viral delivery of DNA encoding editing agents leads to prolonged expression in transduced cells, which increases the occurrence of off-target editing. In addition, viral delivery of DNA raises the possibility of viral vector integration into the genome of transduced cells, both of which can promote oncogenesis or other adverse effects. Further, in spite of the constant evolution of transfection methods and the performance of viral delivery vectors (e.g., AAV or LV), the efficiency of these approaches can vary dramatically, especially in primary cells that are highly sensitive to modifications of their environment and may be altered in response to transfection agents and/or vectors.

More recently, virus-like particles (VLPs) have been engineered to deliver, for example, Cas9 proteins, base editors, and prime editors. See, for example, International Patent Application Nos. PCT/US2022/080834, PCT/US2022/080836, and PCT/US2022/080856, each of which was filed on Dec. 2, 2022, and each of which is incorporated herein by reference. These VLPs allow the direct delivery of ribonucleoproteins (RNPs) (e.g., a gene editing protein agent complexed with a guide RNA) instead of DNA to target cells. The short lifespan of RNPs in cells limits the opportunity for off-target editing. These VLPs, however, utilize wild-type viral proteins, which have been optimized by natural evolution to package viral genomes rather than desired cargo molecules (e.g., gene editing agents such as nucleic acid-programmable DNA-binding proteins (napDNAbps), base editors, and prime editors). Accordingly, there is a need for VLPs that have been optimized specifically to facilitate packaging and delivery of desired cargo molecules, such as gene editing agents.

### SUMMARY OF THE INVENTION

The present disclosure describes the development of a platform/system for the directed evolution of virus-like particles (VLPs) with specific desired properties (e.g., increased production levels, increased cargo packaging efficiency, and/or increased transduction of particular target cell types of interest). This platform was used to evolve several next-generation VLP variants with such desired properties. Because VLPs lack genetic material, traditional directed evolution schemes (such as those commonly used for evolving viruses with desired properties) cannot be used. The platform described herein allows the wild type VLP components, such as the viral nucleocapsid protein, to be evolved to more efficiently package alternative cargo molecules, such as gene editing agents.

As described herein, the inventors discovered that VLP libraries comprising variants of at least one component of the VLP, such as the nucleocapsid protein portion of the gag protein, could be produced in such a way that each VLP variant packages a guide RNA (gRNA) containing a barcode sequence that encodes the identity of that particular VLP variant. In this barcoded VLP library approach, each variant (e.g., nucleocapsid protein variant) is paired with a unique barcode sequence (e.g., as part of a gRNA) on a polynucleotide. The unique barcode sequence (on a gRNA or other nucleic acid molecule) is packaged into the VLP comprising the nucleocapsid protein as it is produced. These barcoded vectors are then used to produce barcoded VLPs in a pooled fashion, ensuring that each VLP variant only packages its cognate barcode, maintaining the specified variant:barcode linkage. After applying a selection for one or more desired properties, the barcoded molecules (e.g., barcoded gRNAs) are sequenced, and the enriched barcodes are quantified (e.g., by sequencing the barcodes and comparing the relative abundance of each), enabling identification of VLPs that possess the desired properties.

The VLPs evolved using the methods provided herein typically comprise a supra-molecular assembly comprising: (a) an envelope comprising (i) a lipid membrane (e.g., single-layer or bi-layer membrane), and a (ii) viral envelope glycoprotein; and (b) a multi-protein core region comprising (ii) a Gag protein, (ii) a first fusion protein comprising a Gag protein and Pro-Pol, and (iii) a second fusion protein comprising a Gag protein fused to a cargo via a protease-cleavable linker.

Thus, in one aspect, the present disclosure provides methods for generating a library of cells capable of producing VLPs comprising transfecting a plurality of polynucleotides encoding VLPs (e.g., the gag-cargo fusion, the gag-pro polyprotein, the envelope glycoprotein, and a barcoded nucleic acid molecule as described herein) into producer cells, wherein each polynucleotide encodes a variant of at least one component of the VLP or its cargo, wherein the variant comprises at least one amino acid substitution relative to a VLP that does not comprise a variant of at least one component of the VLP or its cargo; and wherein each polynucleotide encoding a VLP component or cargo variant is associated with a unique nucleic acid barcode sequence encoded by the polynucleotide (e.g., on a nucleic acid molecule, such as a gRNA, that is packaged into the VLP comprising that particular variant).

In another aspect, the present disclosure provides methods for generating a library of VLPs comprising: (i) transfecting a plurality of polynucleotides encoding VLPs (e.g., the gag-cargo fusion, the gag-pro polyprotein, the envelope glycoprotein, and a barcoded nucleic acid molecule as described herein) into producer cells, wherein each polynucleotide encodes a variant of at least one component of the VLP or its cargo, wherein the variant comprises at least one amino acid substitution relative to a VLP that does not comprise a variant of at least one component of the VLP or its cargo (e.g., that comprises wild type viral proteins); and wherein each polynucleotide encoding a VLP component or cargo variant is associated with a unique nucleic acid barcode sequence encoded by the polynucleotide (e.g., on a nucleic acid molecule, such as a gRNA, that is packaged into the VLP comprising that particular variant); (ii) producing VLPs from the producer cells; and (iii) isolating the library of VLPs.

In another aspect, the present disclosure provides methods for evolving VLPs comprising: (i) transfecting a plurality of polynucleotides encoding VLPs (e.g., the gag-cargo fusion, the gag-pro polyprotein, the envelope glycoprotein, and a barcoded nucleic acid molecule as described herein) into producer cells (e.g., mammalian cells, including human cells, such as Gesicle 293T producer cells), wherein each polynucleotide encodes a variant of at least one component of the VLP or its cargo, wherein the variant comprises at least one amino acid substitution relative to a VLP that does not comprise a variant of at least one component of the VLP or its cargo; and wherein each polynucleotide encoding a VLP component or cargo variant is associated with a unique nucleic acid barcode sequence encoded by the polynucleotide (e.g., on a nucleic acid molecule, such as a gRNA, that is packaged into the VLP comprising that particular variant); (ii) producing VLPs from the producer cells; and (iii) selecting VLPs having at least one improved property by determining the abundance of each barcode sequence relative to the abundance of a barcode sequence associated with a VLP that does not comprise a variant of at least one component of the VLP or its cargo; wherein VLPs comprising variants associated with barcode sequences that are present at higher abundance relative to the barcode sequence associated with the VLP that does not comprise a variant of at least one component of the VLP or its cargo have at least one improved property. In some embodiments, the method is performed one or more, two or more, three or more, four or more, or five or more times to further evolve the VLPs.

In some embodiments, the VLPs evolved in the methods and systems provided herein comprise a variant of a viral nucleocapsid protein, a variant of a viral envelope glycoprotein, and/or a variant of a VLP cargo. In some embodiments, the VLPs evolved using the methods and systems provided herein have higher cargo packaging efficiency. In some embodiments, the VLPs evolved using the methods and systems provided herein are produced at higher levels in producer cells. In some embodiments, the VLPs evolved using the methods and systems provided herein have improved transduction efficiency into target cells.

Once polynucleotides encoding the VLPs have been transfected or transduced into producer cells, it is important that each producer cell is only capable of producing a single VLP library member (e.g., a VLP comprising a particular nucleocapsid protein variant). This ensures that the nucleocapsid protein variant and the barcode will remain associated with one another throughout the selection process since they are expressed from the same polynucleotide. Thus, in some embodiments, a low multiplicity of infection is used when transfecting or transducing the polynucleotides encoding VLPs (e.g., the gag-cargo fusion, the gag-pro polyprotein, the envelope glycoprotein, and a barcoded nucleic acid molecule as described herein) into producer cells, ensuring that producer cells will be capable of encoding either zero or only one VLP variant. In some embodiments, the methods provided herein further comprise a step of selecting for producer cells that contain polynucleotides encoding a VLP. In certain embodiments, such a selection comprises an antibiotic selection.

In some embodiments, the barcode sequence is included on a nucleic acid cargo molecule that is packaged into the VLP. In some embodiments, the nucleic acid cargo molecule is a gRNA. In some embodiments, a barcode sequence on a gRNA is associated with a particular variant of, for example, a viral nucleocapsid protein in a VLP. Such barcode sequences may be included on a plasmid or other vector along with a variant of a VLP component (such as a nucleocapsid protein variant) in the libraries provided herein. Each barcode sequence is thus associated with a particular variant, and as long as the association is maintained throughout the selection process, the barcode sequence will remain associated with a particular variant and allow identification of that variant (e.g., by sequencing) following selection. Such barcodes can be packaged into a VLP as it is produced and then sequenced and used to determine, for example, which VLPs comprise variants of a nucleocapsid protein or other component that confer one or more improved properties relative to a VLP that comprises a wild type nucleocapsid protein or other component of the VLP.

In another aspect, the present disclosure provides libraries of polynucleotides (e.g., plasmids or other vectors) encoding VLPs, wherein each library member comprises a polynucleotide encoding a variant of at least one component of the VLP or its cargo comprising at least one amino acid substitution relative to a VLP that does not comprise a variant of at least one component of the VLP or its cargo, and wherein each polynucleotide encoding a VLP component or cargo variant is associated with a unique nucleic acid barcode sequence encoded by the polynucleotide (e.g., on a nucleic acid molecule, such as a gRNA, that is packaged into the VLP comprising that particular variant). Such libraries may be useful for performing the methods for evolving VLPs described herein.

In another aspect, the present disclosure provides libraries of cells capable of producing virus-like particles (VLPs), wherein each producer cell comprises a polynucleotide encoding a variant of at least one component of a VLP or its cargo comprising at least one amino acid substitution relative to a VLP that does not comprise a variant of at least one component of the VLP or its cargo, and wherein each polynucleotide encoding a VLP component or cargo variant is associated with a unique nucleic acid barcode sequence encoded by the polynucleotide (e.g., on a nucleic acid molecule, such as a gRNA, that is packaged into the VLP comprising that particular variant). Such libraries may be useful for performing the methods for evolving VLPs described herein. In some embodiments, the cells are human cells. In some embodiments, the cells are mammalian cells. In certain embodiments, the cells are Gesicle 293T producer cells.

In some aspects, the present disclosure also provides gag proteins comprising a nucleocapsid protein variant. In some aspects, the present disclosure provides VLPs evolved using the methods provided herein.

In one aspect, the present disclosure provides group specific antigen (gag) proteins comprising viral nucleocapsid protein variants evolved using the methods described herein.

In some embodiments, the gag protein (which comprises the viral nucleocapsid protein) is a variant that is at least 70%, at least 75%, at least 80%, at least 85%, at least 90%, at least 95%, at least 96%, at least 97%, at least 98%, or at least 99% identical to the amino acid sequence of a gag protein of SEQ ID NO: 100 and comprises one or more substitutions at positions selected from the group consisting of 215, 216, 218, 219, 220, 221, 222, 223, 224, 225, 226, 227, 228, 229, 230, 231, 232, 233, 234, 235, 236, 237, 238, 239, 240, 241, 242, 244, 245, 246, 249, 250, 253, 254, 255, 256, 257, 258, 259, 260, 261, 262, 263, 264, 265, 266, 269, 271, 272, 274, 275, 276, 277, 279, 280, 281, 282, 283, 285, 286, 288, 289, 291, 292, 293, 294, 295, 296, 297, 298, 299, 300, 301, 302, 303, 304, 305, 307, 308, 310, 311, 418, 420, 421, 424, 427, 430, 432, 433, 435, 436, 437, 438, 440, 441, 443, 444, 446, 447, 448, 449, 452, 455, 458, 460, 463, 464, 466, 467, 468, 469, 470, 471, 472, 473, 474, 475, 477, 478, 479, 480, 481, 482, 483, 485, 486, 487, 488, 489, 490, 491, 492, 495, 496, 497, 498, 499, 500, 501, 502, 503, 504, 505, 506, 507, 508, 509, 510, 511, and 512 relative to the amino acid sequence provided in SEQ ID NO: 100, or at corresponding positions in a homologous viral nucleocapsid protein.

In certain embodiments, the amino acid sequence of the gag protein comprising a viral nucleocapsid protein variant comprises one or more substitutions selected from the group consisting of F215L, F215G, F215M, P216K, P216I, R218I, R218G, R218H, A219C, A219K, A219N, A219G, G220C, G221W, G221M, N222H, N2221, N222M, N222Y, N222V, G223K, G223A, G223C, G223D, Q224L, Q224R, Q224F, Q2241, L225Q, Q226P, Y227S, W228N, W228F, P229D, P229L, F230C, F230E, S231Y, S23iF, S232L, S232A, S233K, S233I, S233R, D234E, D234A, L235C, L235M, Y236E, Y236M, N237D, W238D, K239A, K239T, K239H, N240A, N240E, N240L, N240S, N240I, N241K, N241V, N242T, S244Y, S244M, S244T, F245H, F245R, F245W, S246L, S246H, S246F, S246V, S246Y, P249S, P249F, P249K, G250C, G250D, G250L, G250R, T253F, A254W, L255H, L255V, I256V, I256W, E257A, E257C, S258W, S258V, V259R, L260M, L260W, L2601, 1261K, I261W, I261Q, T262N, T262W, T262F, T262Q, H263I, Q264S, Q264T, P265G, P265F, T266C, D269G, Q27iF, Q271A, Q271D, Q272P, Q272G, L274W, G275W, T276R, T276V, T276M, L277W, T279G, G280W, G280D, G280Y, E281T, E281N, E281C, E282S, E282H, E282Y, K283F, R285V, V286H, L288D, L288K, L288A, E289C, R291K, K292L, A293H, A293Y, V294Q, R295M, G296D, D297N, D297A, D297M, D297P, D297W, D298Y, G299M, G299R, G299Y, R300L, P301S, P301L, T302V, Q303S, L304N, P305R, P305G, P305M, E307N, V308R, V308I, A310R, A311F, A311E, A311M, K418Y, L420C, G421D, G421A, G421R, V424Y, A427G, I430W, I430H, N432Y, K433P, E435A, T436N, P437K, E438H, R440C, R440P, E441M, R443P, I444E, R446G, R446I, E447H, E447S, T448M, T448W, T448V, E449D, E452S, R455Q, E458F, E460W, E463H, K464L, R466Q, R466A, D467E, R468Q, R468W, R468I, R468S, R469Q, R470A, R470M, R470W, H471M, H471D, H471G, H47iN, R472E, R472D, E473N, M474W, M474C, M474I, S475G, L477W, L477C, L477E, L478K, L478V, L478P, A479Q, A479L, A479K, A479S, A479Y, T480H, T480W, T480E, V48iT, V482L, V482M, S483W, S483L, S483R, Q485K, Q485G, Q485L, Q485W, K486L, K486I, K486V, K486E, K486F, Q487P, Q487E, D488Q, D488I, R489A, R489K, R489M, R489F, Q490T, G491C, G491Q, G491H, G492Q, G492W, G492H, R495Y, R495K, R495F, R495I, R496L, R496N, R496F, R496A, R496K, S497C, S497P, S497Q, S497T, S497N, Q498K, Q498V, L499Y, L499F, L499G, L499T, D500Q, D500G, D500M, D500I, R501D, R501I, R501L, D502Q, D502A, D502P, Q503T, C504N, C504S, A505M, A505Y, A505W, A505I, Y506L, Y506M, C507F, C507V, C507G, C507W, K508D, K508N, E509G, E509A, E509M, K510M, K510P, K510R, G511L, G511K, G511P, H512Q, H512S, and H512M relative to the amino acid sequence provided in SEQ ID NO: 100, or corresponding substitutions in a homologous viral nucleocapsid protein.

In some embodiments, the amino acid sequence of the gag protein comprising a viral nucleocapsid protein variant comprises one or more substitutions at positions which result in a VLP comprising the variant being produced at higher levels and/or packaging a cargo molecule more efficiently, as compared to a VLP comprising a nucleocapsid protein that does not comprise these mutations. In some embodiments, the nucleocapsid protein variant comprises one or more substitutions at positions selected from the group consisting of 222, 226, 227, 228, 229, 230, 235, 239, 240, 244, 245, 246, 253, 254, 256, 260, 261, 272, 276, 277, 279, 292, 293, 297, 305, 308, 418, 427, 440, 443, 463, 466, 467, 470, 471, 472, 473, 477, 478, 479, 482, 483, 485, 496, 497, 499, 500, 505, 506, and 507 relative to the amino acid sequence provided in SEQ ID NO: 100, or at corresponding positions in a homologous viral nucleocapsid protein. In certain embodiments, the nucleocapsid protein variant comprises one or more substitutions selected from the group consisting of N222H, N222I, Q226P, Y227S, W228N, P229D, F230C, L235C, K239A, N240A, S244Y, F245H, F245R, S246L, S246H, T253F, A254W, 1256V, L260M, 1261K, Q272P, T276R, L277W, T279G, K292L, A293H, D297N, D297A, P305R, V308R, K418Y, A427G, R440P, R443P, E463H, R466Q, D467E, R470A, H471M, R472E, E473N, L477W, L478K, A479Q, A479L, V482M, S483W, Q485K, R496L, S497C, L499Y, L499F, D500Q, A505M, Y506L, and C507F relative to the amino acid sequence provided in SEQ ID NO: 100, or corresponding substitutions in a homologous viral nucleocapsid protein. When a gag protein comprising any of these amino acid substitutions is included in a VLP, the VLP may be produced at higher levels and/or package a cargo molecule more efficiently as compared to a VLP comprising a gag protein that does not comprise these amino acid substitutions.

In some embodiments, the amino acid sequence of the gag protein comprising a viral nucleocapsid protein variant comprises one or more substitutions at positions which result in a VLP comprising the variant having a higher transduction efficiency into target cells, as compared to a VLP comprising a nucleocapsid protein that does not comprise these mutations. In some embodiments, the nucleocapsid protein variant comprises one or more substitutions at positions selected from the group consisting of 215, 226, 257, 293, 467, 482, 485, and 507 relative to the amino acid sequence provided in SEQ ID NO: 100, or at corresponding positions in a homologous viral nucleocapsid protein. In certain embodiments, the nucleocapsid protein variant comprises one or more substitutions selected from the group consisting of F215G, Q226P, E257C, A293Y, D467E, V482M, Q485K, and C507V relative to the amino acid sequence provided in SEQ ID NO: 100, or corresponding substitutions in a homologous viral nucleocapsid protein. In some embodiments, the nucleocapsid protein variant comprises one or more substitutions at positions selected from the group consisting of 215, 219, 226, 261, 272, 280, 283, 288, 310, 469, 471, 472, 478, 479, 480, 485, 490, 492, 496, 497, 500, 501, 502, 504, 505, 507, and 508 relative to the amino acid sequence provided in SEQ ID NO: 100, or at corresponding positions in a homologous viral nucleocapsid protein. In certain embodiments, the nucleocapsid protein variant comprises one or more substitutions selected from the group consisting of F215G, A219C, Q226P, I261W, Q272G, G280W, K283F, L288A, A310R, R469Q, H471D, H471M, R472E, L478K, A479K, T480H, Q485L, Q490T, G492W, R496F, S497Q, D500Q, R501D, R501I, D502Q, C504N, A505M, A505W, C507F, C507V, and K508D relative to the amino acid sequence provided in SEQ ID NO: 100, or corresponding substitutions in a homologous viral nucleocapsid protein. When a gag protein comprising any of these amino acid substitutions is included in a VLP, the VLP may have a higher transduction efficiency into target cells as compared to a VLP comprising a gag protein that does not comprise these amino acid substitutions.

In some embodiments, the present disclosure comprises gag proteins comprising one or more, two or more, three or more, four or more, or five or more substitutions at positions selected from the group consisting of 215, 219, 226, 233, 255, 256, 260, 261, 272, 280, 283, 288, 310, 440, 443, 444, 469, 471, 472, 478, 479, 480, 481, 485, 490, 492, 496, 497, 500, 501, 502, 505, and 507, optionally wherein when incorporated into a VLP the gag protein facilitates higher packaging of one or more cargo molecules in the VLP and/or increased production of the VLP in a producer cell, and/or optionally wherein when incorporated into a VLP the gag protein facilitates improved transduction efficiency of the VLP into a target cell type. In certain embodiments, the gag protein comprises one or more, two or more, three or more, four or more, or five or more of the amino acid substitutions F215G, A219C, Q226P, S233K, L255V, I256W, L2601, 1261W, Q272G, G280W, K283F, L288A, A310R, R440P, R443P, I444E, R469Q, H471D, H471M, R472E, L478K, A479K, T480H, V48iT, Q485L, Q490T, G492W, R496F, S497Q, D500Q, R501I, D502Q, A505M, A505W, C507F, and C507V, optionally wherein when incorporated into a VLP the gag protein facilitates higher packaging of one or more cargo molecules in the VLP and/or increased production of the VLP in a producer cell, and/or optionally wherein when incorporated into a VLP the gag protein facilitates improved transduction efficiency of the VLP into a target cell type. In certain embodiments, the gag protein comprises one or more, two or more, three or more, four or more, or five or more of the amino acid substitutions F215G, Q226P, L260I, G280W, A310R, 1444E, L478K, A479K, T480H, V481T, Q490T, G492W, A505W, and C507V, wherein when incorporated into a VLP the gag protein facilitates higher packaging of one or more cargo molecules in the VLP and/or increased production of the VLP in a producer cell. In certain embodiments, the gag protein comprises one or more, two or more, three or more, four or more, or five or more of the amino acid substitutions L255V, I256W, L260I, L288A, R440P, and R443P, wherein when incorporated into a VLP the gag protein facilitates improved transduction efficiency of the VLP into a target cell type.

In another aspect, the present disclosure provides VLPs comprising any of the gag proteins comprising a nucleocapsid protein variant (or other VLP component variants) provided herein. In some embodiments, the nucleocapsid protein variant is incorporated into the gag protein of the gag-cargo fusion protein component of the VLP. In some embodiments, the nucleocapsid protein variant is incorporated into the gag protein of the gag-pro polyprotein component of the VLP. In certain embodiments, the gag protein of the gag-pro polyprotein of the VLP comprises an amino acid substitution at position 226 (e.g., Q226P) relative to the amino acid sequence provided in SEQ ID NO: 100, or at a corresponding position in a homologous viral nucleocapsid protein; and the gag protein of the gag fusion protein of the VLP comprises a substitution at amino acid position 501 (e.g., R501D) relative to the amino acid sequence provided in SEQ ID NO: 100, or at a corresponding position in a homologous viral nucleocapsid protein. In certain embodiments, the gag protein of the gag-pro polyprotein of the VLP comprises a substitution at amino acid position 501 (e.g., R501I) relative to the amino acid sequence provided in SEQ ID NO: 100, or at a corresponding position in a homologous viral nucleocapsid protein; and the gag protein of the gag fusion protein of the VLP comprises substitutions at amino acid positions 226 and 501 (e.g., Q226P and R501I) relative to the amino acid sequence provided in SEQ ID NO: 100, or at corresponding positions in a homologous viral nucleocapsid protein. In certain embodiments, the gag protein of the gag-pro polyprotein comprises a substitution at amino acid position 226 (e.g., Q226P) relative to the amino acid sequence provided in SEQ ID NO: 100, or at a corresponding position in a homologous viral nucleocapsid protein; and the gag protein of the gag fusion protein comprises a substitution at amino acid position 507 (e.g., C507V) relative to the amino acid sequence provided in SEQ ID NO: 100, or at a corresponding position in a homologous viral nucleocapsid protein.

In another aspect, the present disclosure provides polynucleotides encoding any of the gag protein variants (or other VLP component variants) or VLPs provided herein. In another aspect, the present disclosure provides vectors comprising any of the polynucleotides provided herein.

In another aspect, the present disclosure provides pharmaceutical compositions comprising any of the gag protein variants (or other VLP component variants), VLPs, polynucleotides, or vectors provided herein.

In another aspect, the present disclosure provides cells comprising any of the gag protein variants (or other VLP component variants), VLPs, polynucleotides, or vectors provided herein.

In another aspect, the present disclosure provides methods comprising transfecting or transducing a target cell with any of the VLPs provided herein.

In another aspect, the present disclosure provides for the use of any of the gag protein variants (or other VLP component variants), VLPs, polynucleotides, vectors, or cells provided herein in medicine.

In another aspect, the present disclosure provides for the use of any of the gag protein variants (or other VLP component variants), VLPs, polynucleotides, vectors, or cells provided herein in the manufacture of a medicament.

In another aspect, the present disclosure provides kits comprising any of the libraries, polynucleotides, compositions, cells, or VLPs provided herein.

In another aspect, the present disclosure provides systems of polynucleotides comprising (i) a first polynucleotide encoding a gag protein-cargo fusion and a nucleic acid molecule comprising a unique barcode sequence; (ii) a second polynucleotide encoding a viral envelope glycoprotein; and (iii) a third polynucleotide encoding a gag-pro polyprotein.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying Figures.

### BRIEF DESCRIPTION OF THE DRAWINGS

The following Figures form part of the present specification and are included to further demonstrate certain aspects of the present disclosure, which can be better understood by reference to one or more of these drawings in combination with the detailed description of specific embodiments presented herein.

FIG. 1 shows development of next-generation eVLPs through directed evolution.

FIG. 2 shows challenges associated with evolving eVLPs due to their lack of genetic material and a strategy for performing selections with barcoded eVLP libraries. Top, standard scheme for the directed evolution of viruses, in which the packaged viral genome encodes the viral variant. Bottom, new scheme for the directed evolution of eVLPs, in which the packaged sgRNA barcodes encode the eVLP variant.

FIG. 3 shows the use of barcoded sgRNAs to enable the identification of capsid variants. Each eVLP variant was linked to a unique barcoded sgRNA during library construction. This linkage was maintained during eVLP production so that the identity of a particular eVLP could be determined by sequencing the identity of the sgRNA barcodes packaged within that eVLP.

FIG. 4 shows key features of a barcoded eVLP evolution platform.

FIGS. 5A-5B show that eVLPs tolerated barcoded sgRNAs and were produced by both a four-plasmid system (FIG. 5A) and a three-plasmid system (FIG. 5B). Adenine base editing efficiencies in HEK293T cells transduced by different barcode-containing eVLP constructs are shown.

FIG. 6 shows that maintaining barcode linkage during eVLP production required only one library member per producer cell.

FIG. 7 provides an optimized scheme for maintaining barcode linkage during eVLP production. Plasmid libraries were used to generate lentiviral libraries, which were then used to generate an eVLP producer cell library such that each producer cell contained an integrated copy of a single eVLP variant. This producer cell library was then used to generate a barcoded eVLP library in which barcode/variant linkage was faithfully maintained.

FIG. 8 shows validation that the barcode linkage was maintained. A mock selection scheme with two barcoded variants and lentiviral library generation was provided.

FIGS. 9A-9B show further validation that the barcode linkage was maintained using an optimized protocol for sgRNA extraction from eVLPs, an optimized targeted reverse transcription reaction, and sequencing analysis to determine barcode enrichment frequencies. Observed barcode frequency in the input population and post-selection (in eVLPs) is shown (FIG. 9B). In FIG. 9A, sequences shown correspond (top-bottom) to SEQ ID NOs: 101-104.

FIG. 10 provides a schematic showing cloning of a barcoded capsid library. Top, barcode and capsid mutant correspondence was defined by synthesizing specific barcode/mutant pairs on the same oligonucleotide. Amplified oligonucleotide pools were cloned via Gibson assembly into an appropriate acceptor vector. Bottom, in a second step, the intervening gene and promoter sequences (which are constant for all library members) were inserted into the library vectors using Golden Gate assembly.

FIG. 11 shows library validation to ensure coverage of all barcodes. Frequency of barcode sequences observed in a representative 1,000-member sub-library using high-throughput sequencing is shown.

FIGS. 12A-12B provide schematics showing screening of a barcoded eVLP library for improved eVLP production efficiency. Barcode enrichment in eVLPs relative to producer cells reveals how different capsid mutants influence eVLP production.

FIG. 13 shows that the barcode distribution in producer cell genomic DNA indicates coverage of 99% of library members. Integrated barcode loci were amplified from producer cell genomic DNA and subjected to high-throughput sequencing.

FIGS. 14A-14B show that screening of barcoded eVLPs revealed capsid mutants with improved properties as described herein.

FIGS. 15A-15D show analysis of eVLP capsid mutants discovered from screening.

FIG. 15A shows barcode enrichment in the eVLP production screen. Barcode enrichment in eVLPs relative to producer cell gDNA was observed in two replicates of the production screen. The value corresponding to the wild-type v4 eVLP capsid is shown as a dot labeled “WT.” The box encompasses values corresponding to capsid mutants that enriched above wild-type levels in both replicates. FIG. 15B shows screening of eVLP capsid variants for improved transduction. Left, schematic of barcoded eVLP screen for improved transduction.

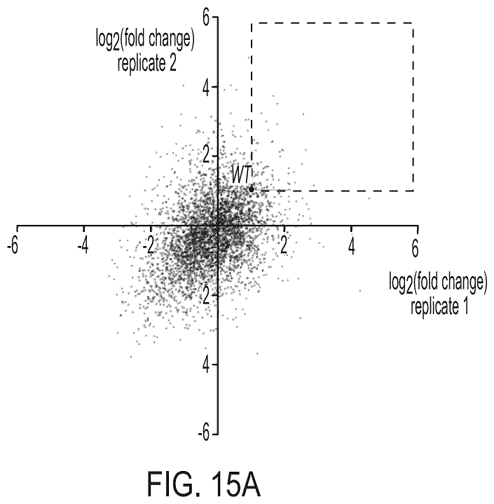

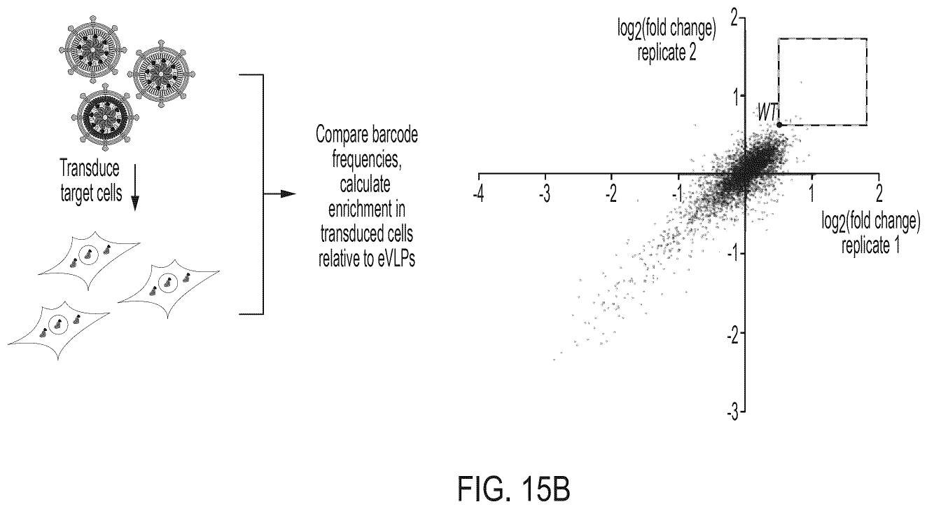

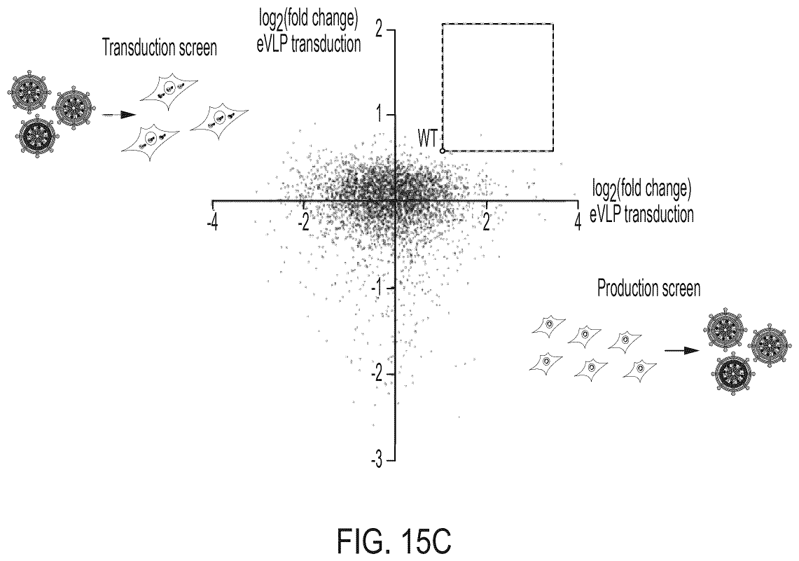

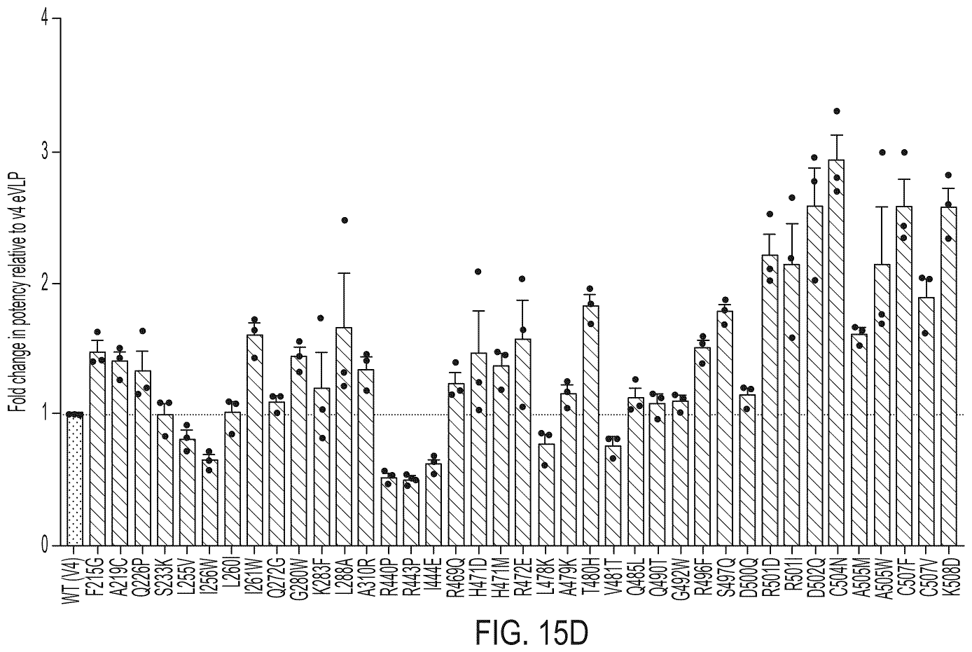

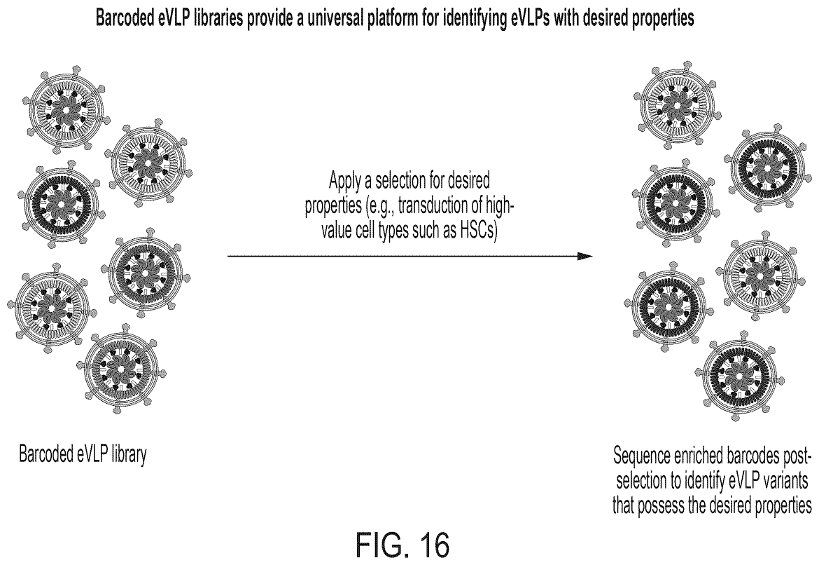

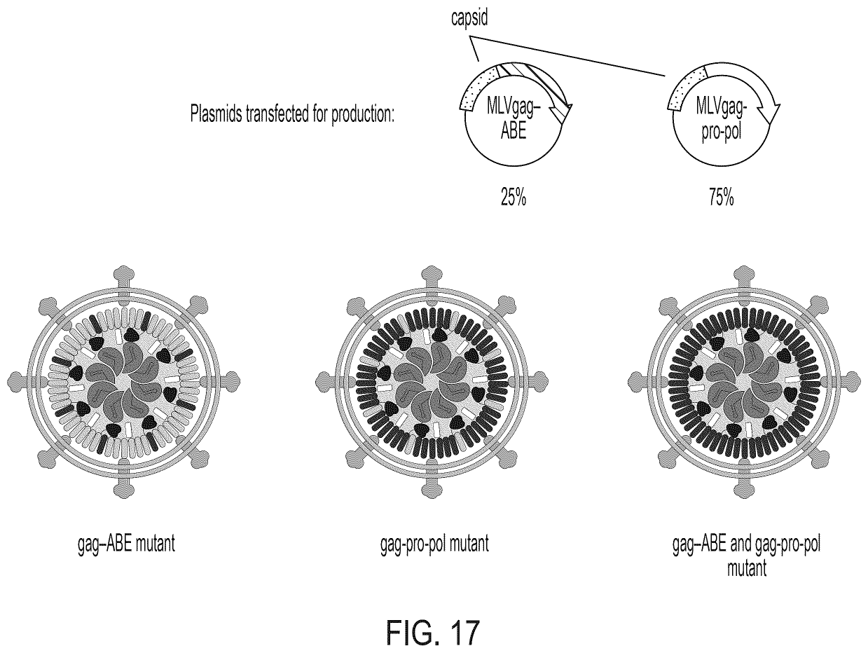

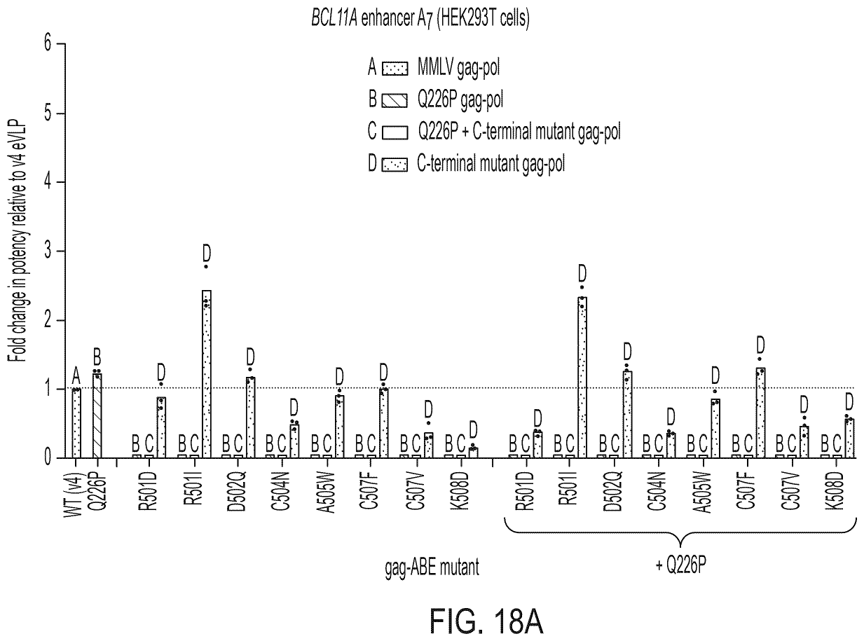

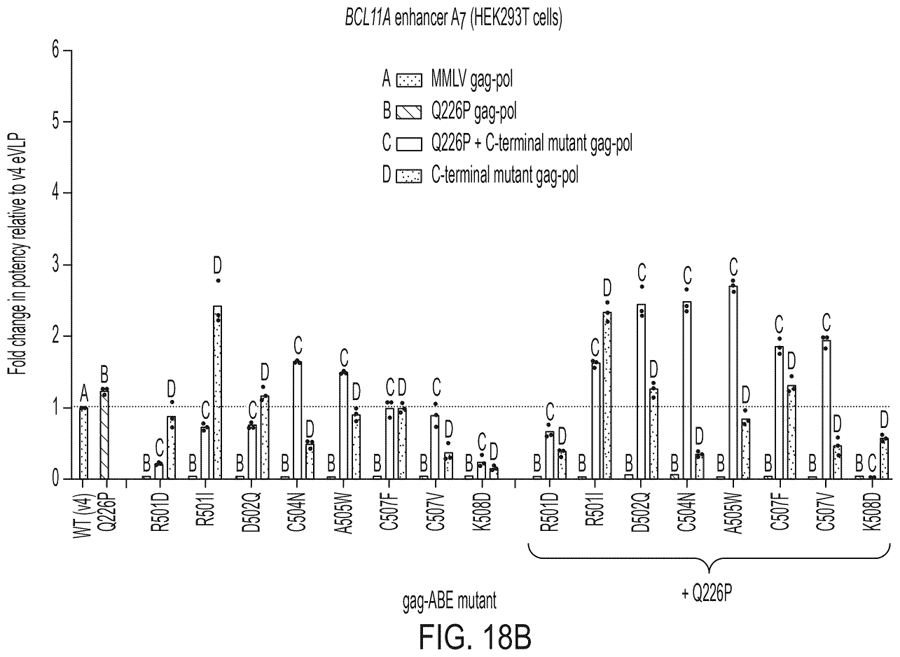

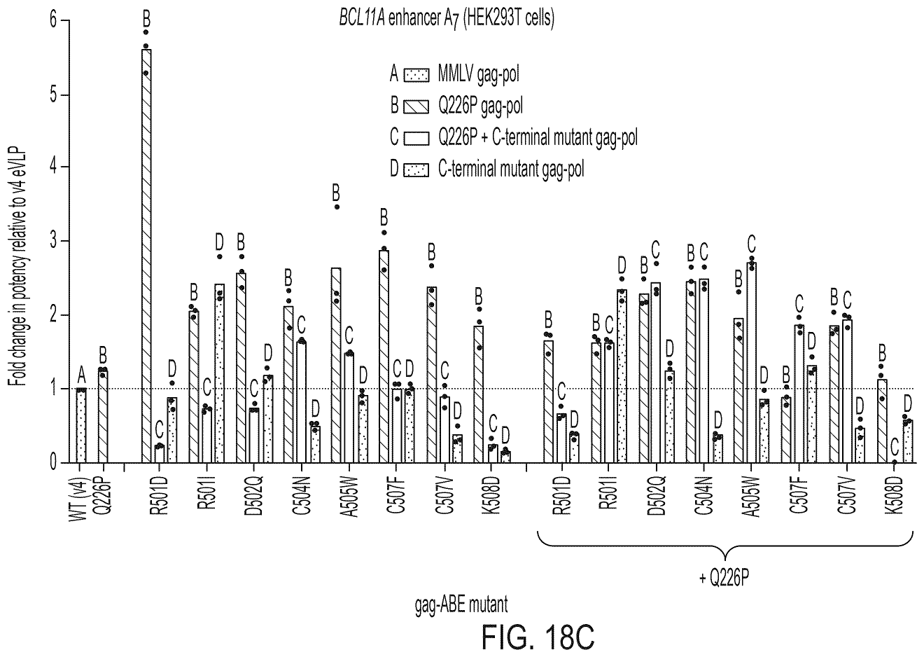

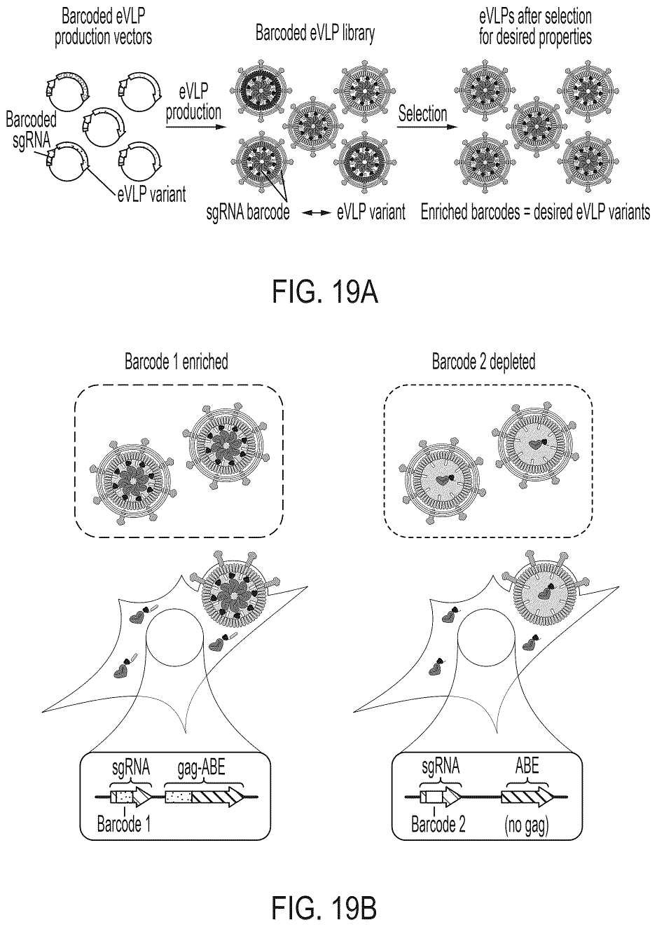

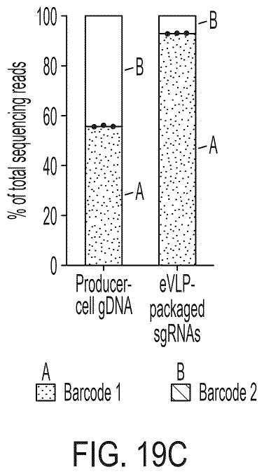

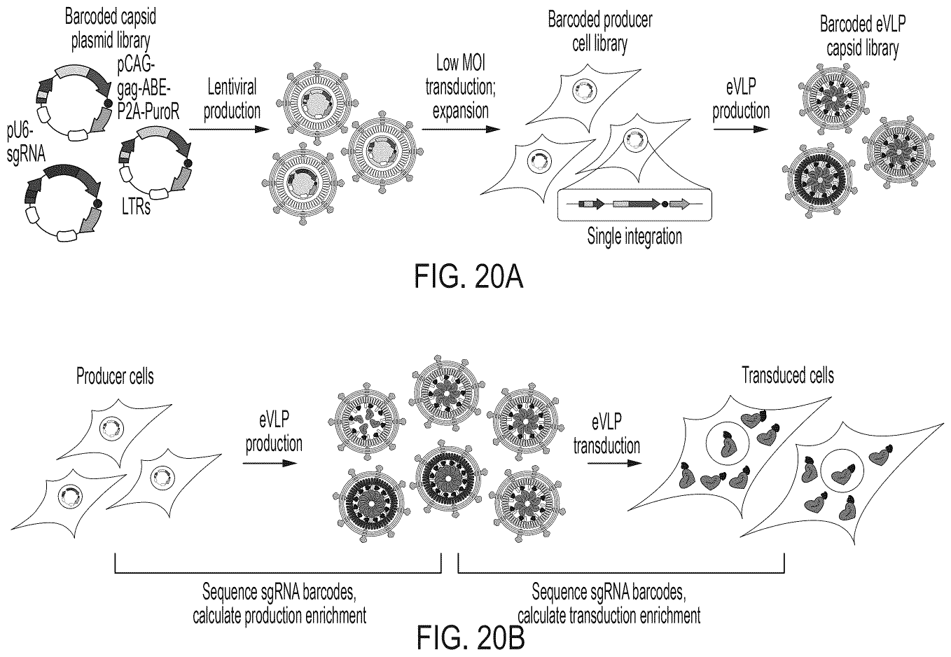

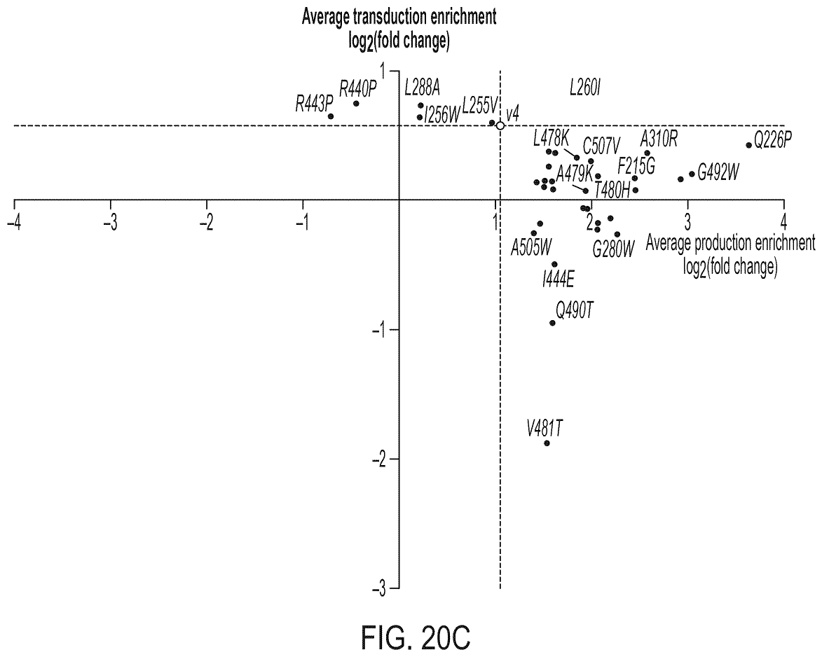

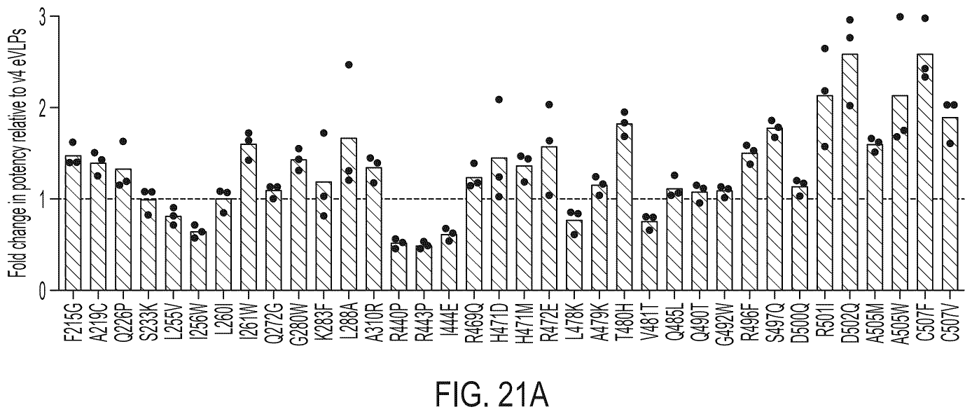

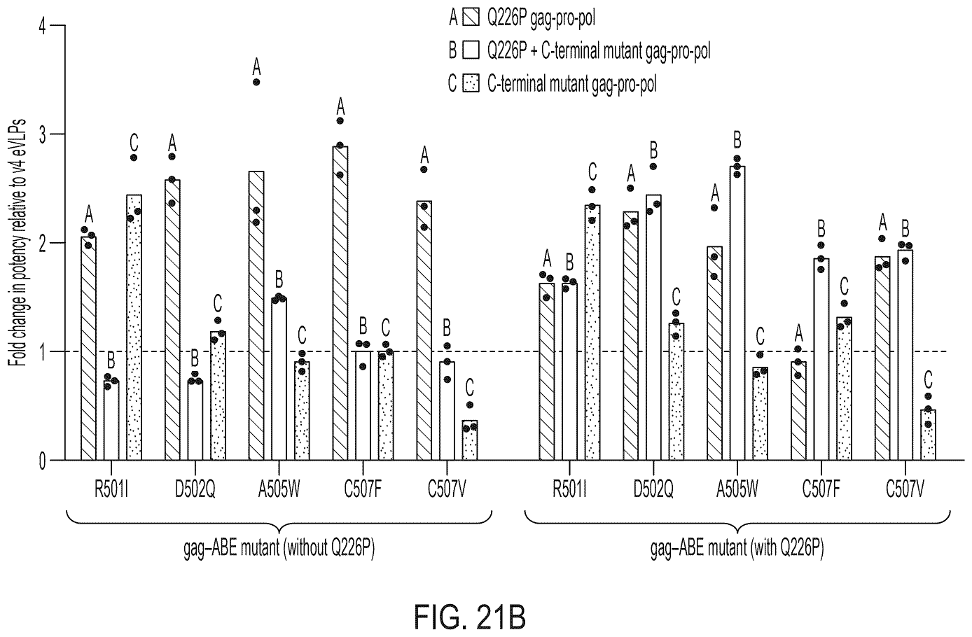

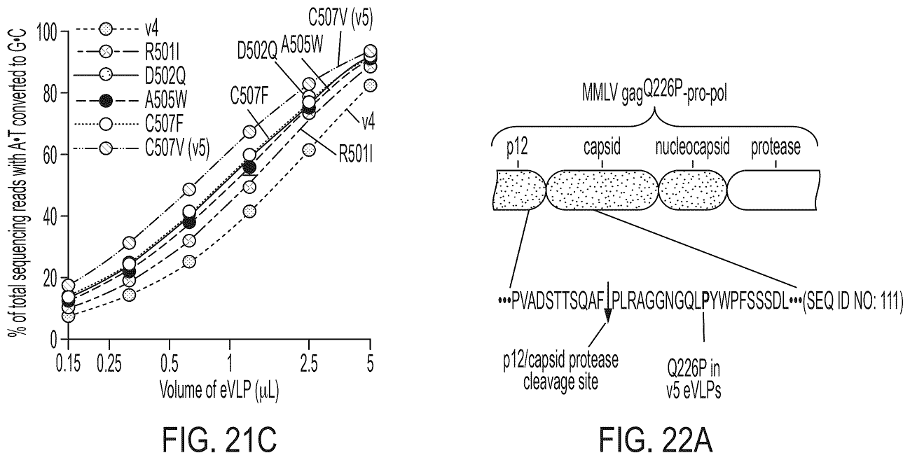

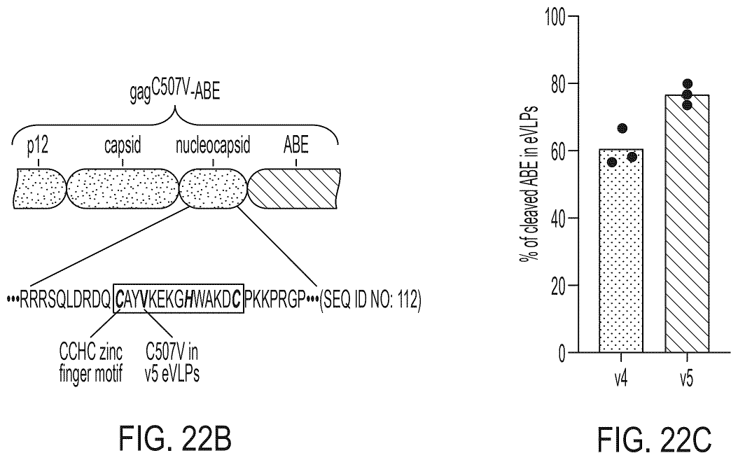

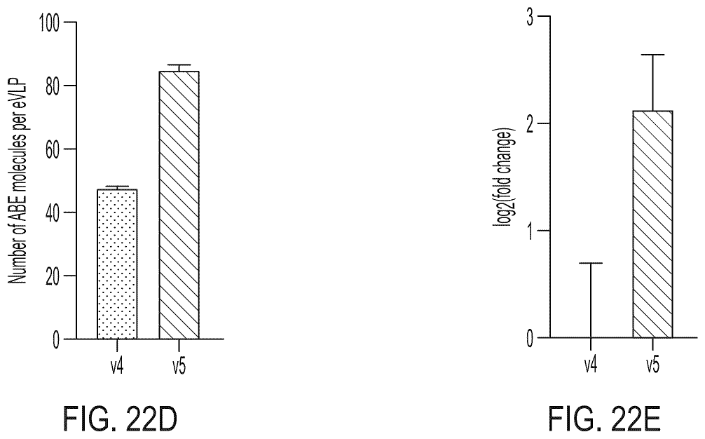

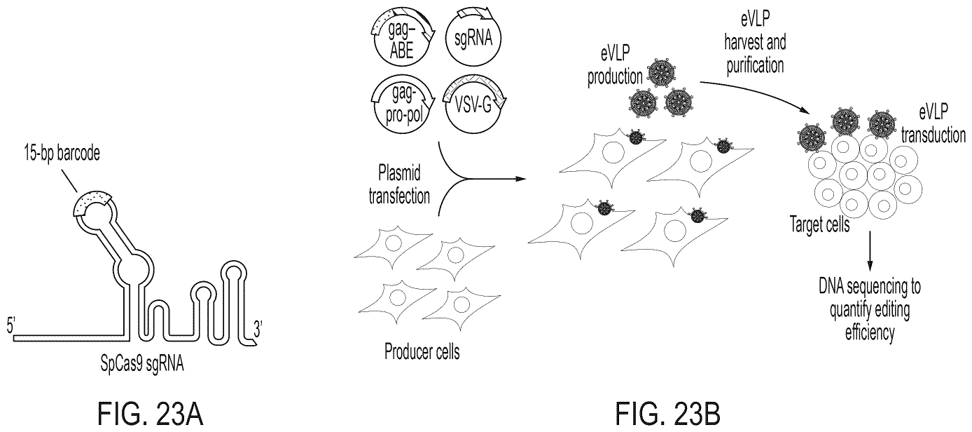

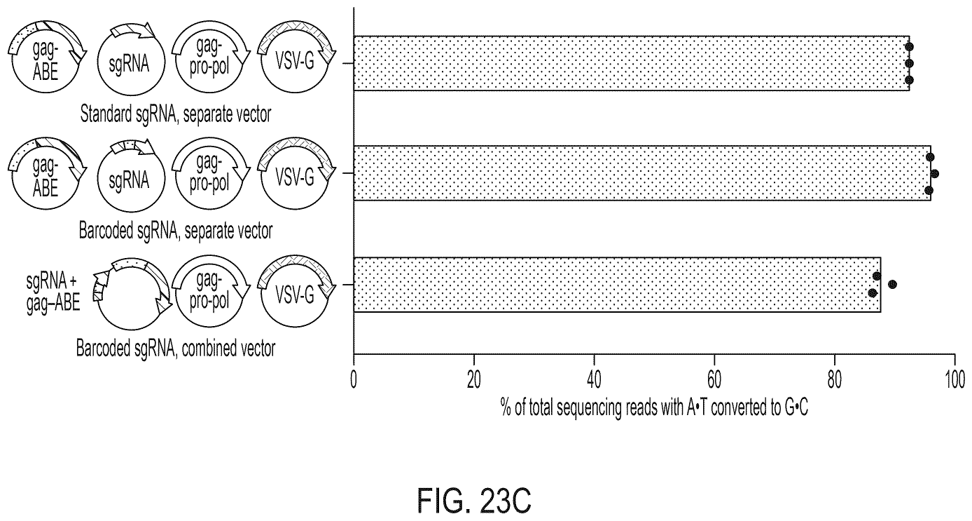

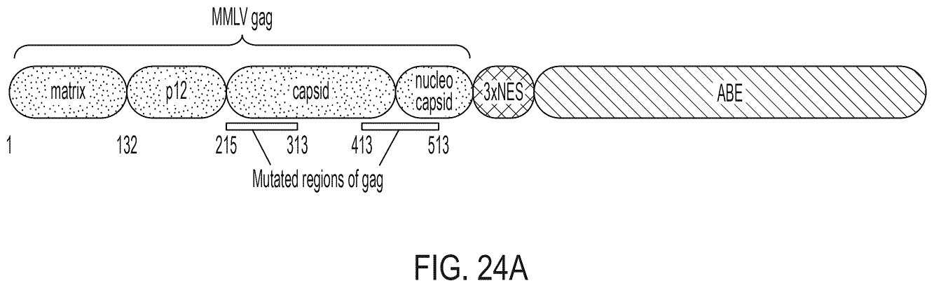

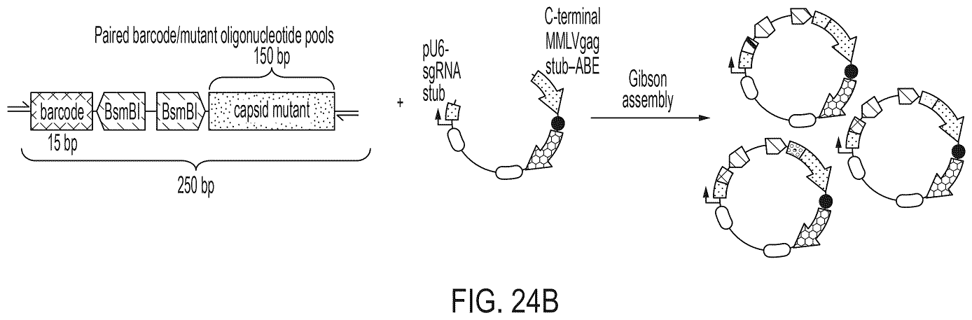

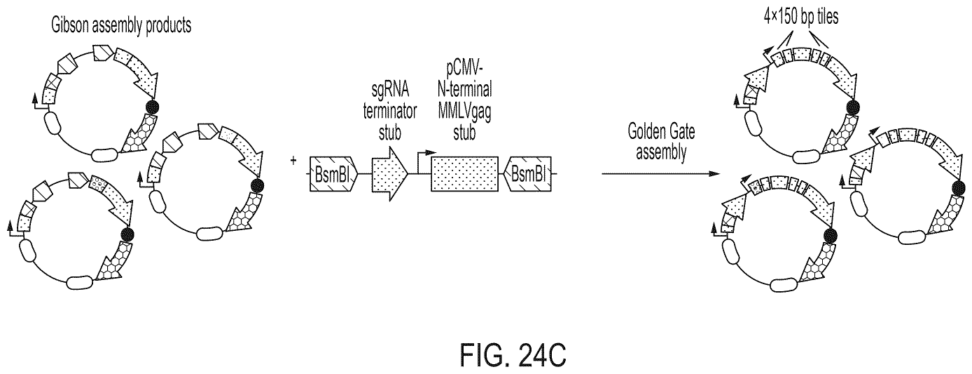

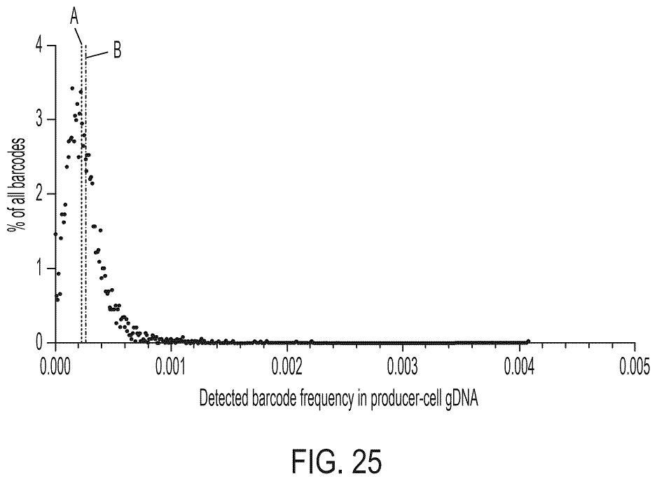

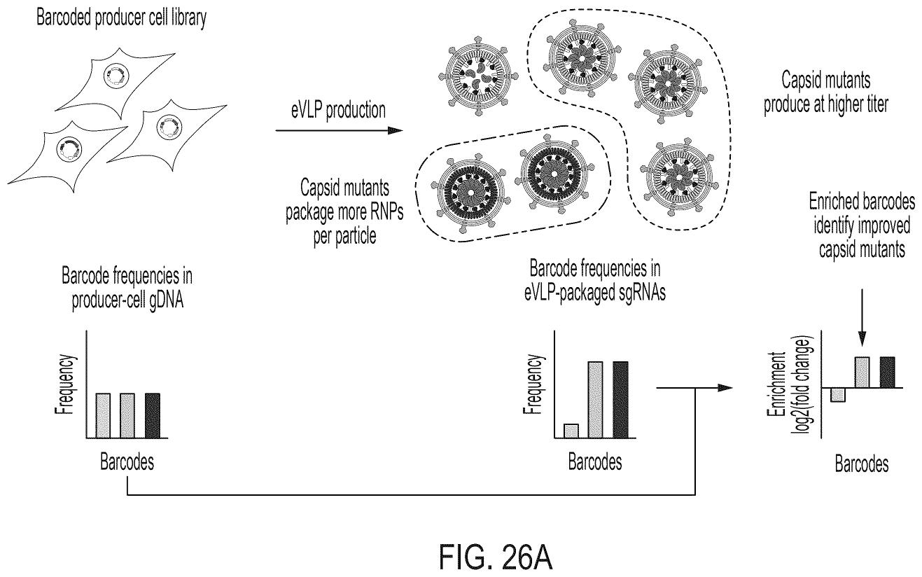

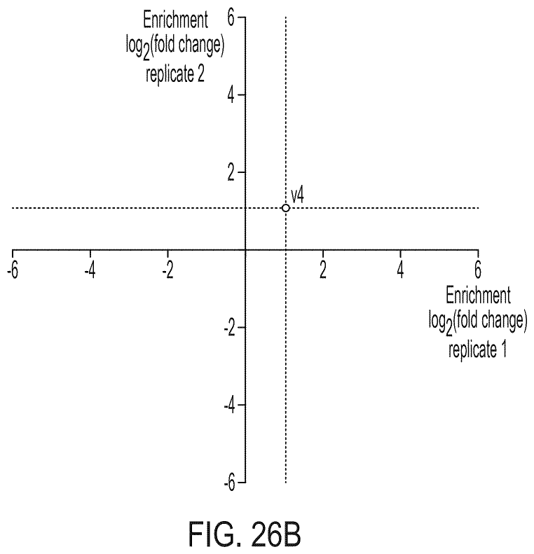

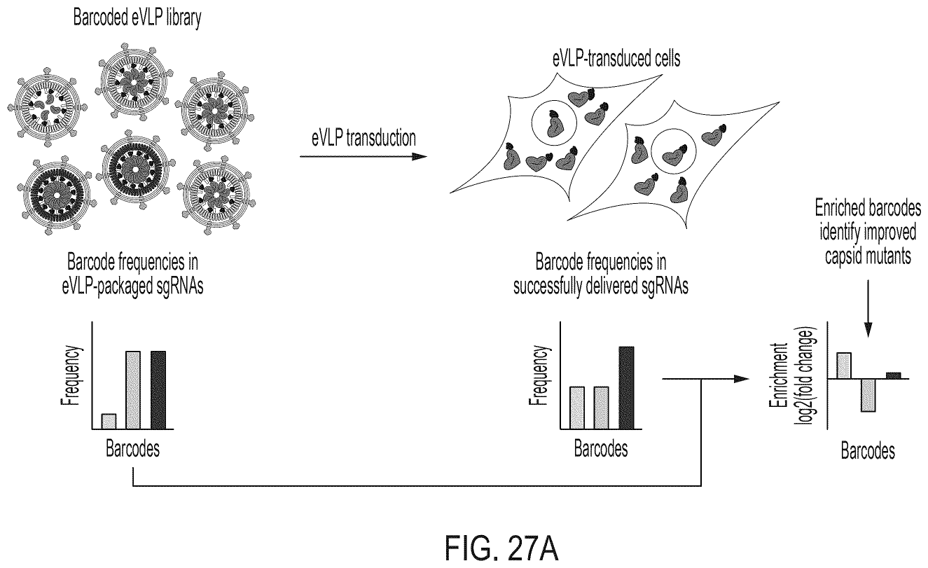

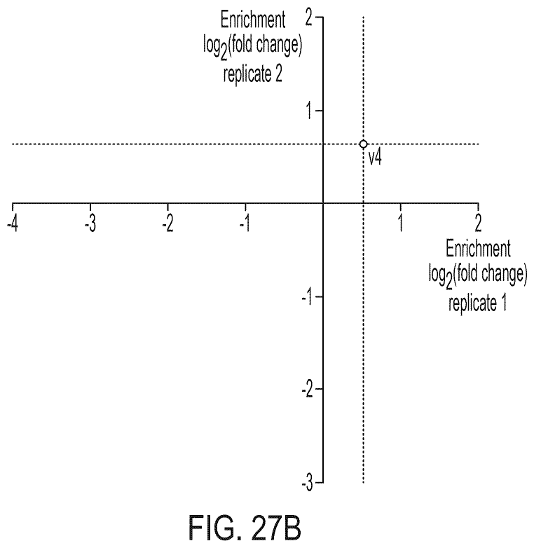

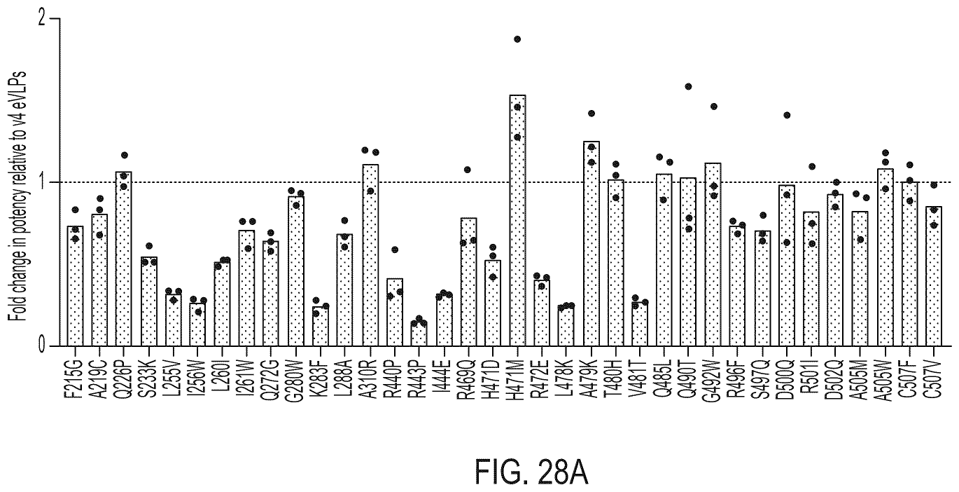

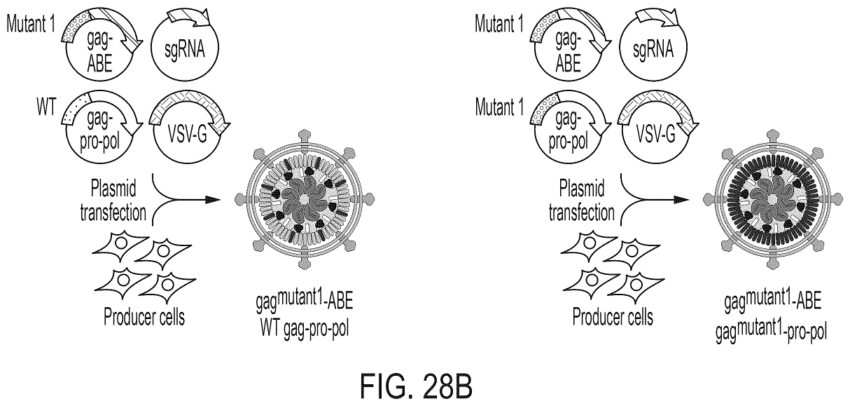

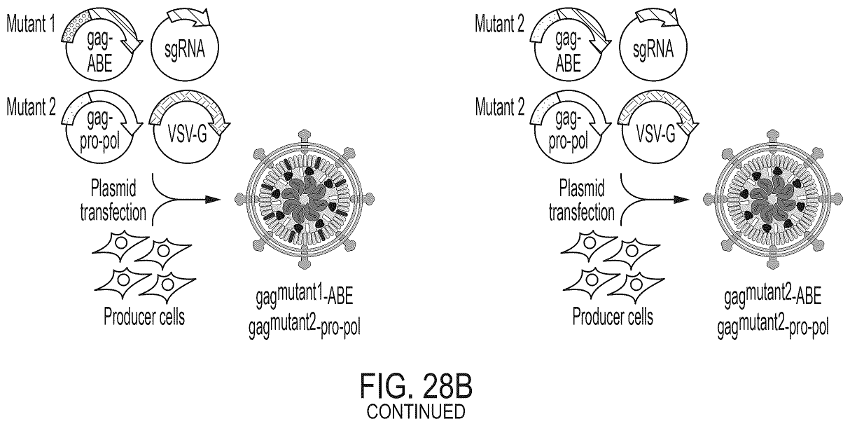

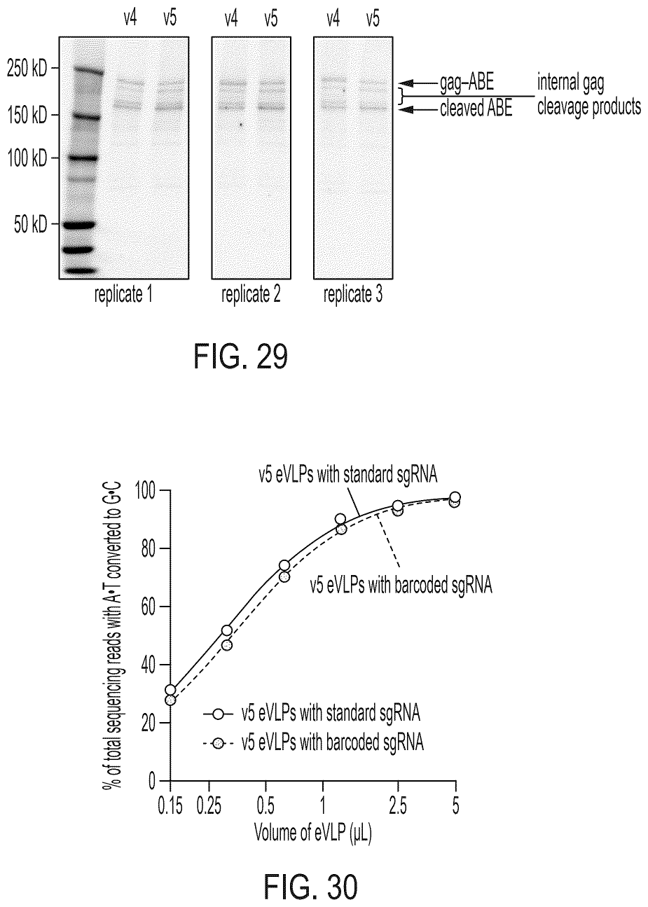

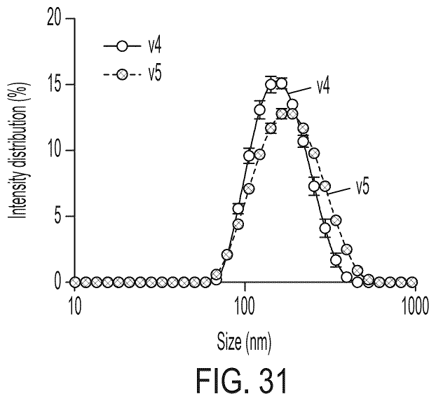

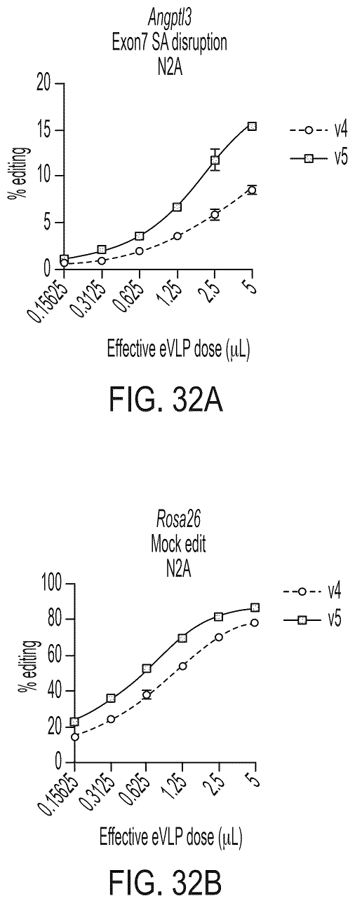

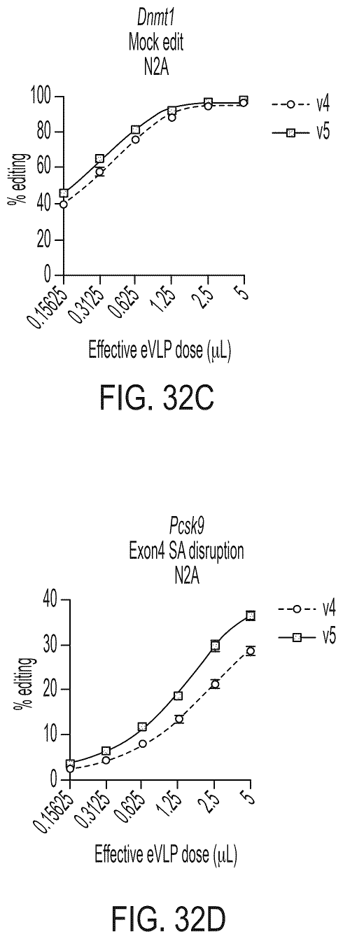

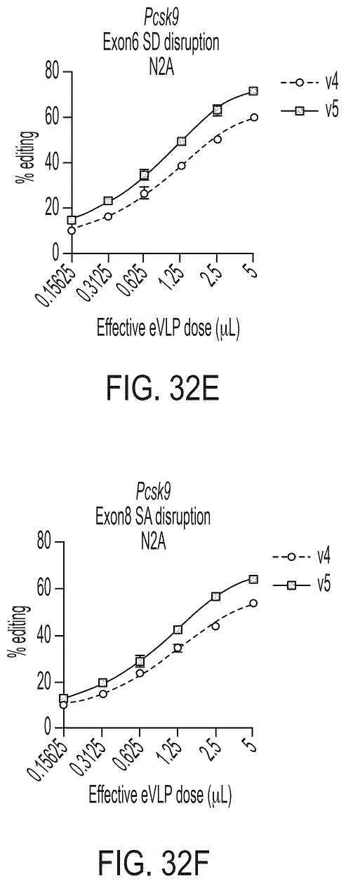

Right, barcode enrichment in transduced cells relative to eVLPs was observed in two replicates of the transduction screen. The value corresponding to the wild-type v4 eVLP capsid is shown as a dot labeled “WT.” The box encompasses values corresponding to capsid mutants that enriched above wild-type levels in both replicates. FIG. 15C shows barcode enrichment in production screens vs. transduction screens. Each capsid variant is plotted as a single dot. The x-coordinate corresponds to that capsid variant's average enrichment value in the production screen, and the y-coordinate corresponds to that capsid variant's average enrichment value in the transduction screen. The value corresponding to the wild-type v4 eVLP capsid is shown as a dot labeled “WT.” The box encompasses values corresponding to capsid mutants that enriched above wild-type levels in both the production and transduction screens. FIG. 15D shows potencies of screen-enriched capsid mutants. The potencies of capsid mutants that enriched in either production or transduction screens, assayed individually, are shown. Fold change in potency is defined as fold change in observed editing efficiency at the BCL11A enhancer locus.

FIG. 16 provides a schematic showing that barcoded eVLP libraries provide a universal platform for identifying eVLPs with desired properties.

FIG. 17 shows that the identified nucleocapsid mutants were incorporated into both the gag-pro-polyprotein and/or the gag-ABE fusion protein.

FIGS. 18A-18C show the potency of selected mutations incorporated into the gag-ABE fusion protein vs. the gag-pro-polyprotein.

FIGS. 19A-19C show validation of the barcoded eVLP evolution system. FIG. 19A shows an overview of the barcoded eVLP evolution system. Each unique eVLP variant was linked to a unique barcoded sgRNA on the same eVLP production vector. eVLP production maintained barcode/variant correspondence and resulted in a barcoded eVLP library in which each eVLP variant packaged RNPs containing barcoded sgRNAs that encoded the identity of that particular eVLP variant. Barcodes that were enriched following a selection for desired properties identified eVLP variants that possessed the desired properties. FIG. 19B shows a schematic of the mock selection experiment with barcode 1 linked to a functional gag-ABE construct and barcode 2 linked to a non-functional ABE only (no gag) construct. FIG. 19C shows the frequencies of barcodes 1 or 2 detected in either the producer-cell gDNA or the eVLP-packaged sgRNAs. Bars reflect the mean of n=3 biological replicates, and dots represent individual replicate values. ABE indicates adenine base editor, and gDNA indicates genomic DNA.

FIGS. 20A-20C show barcoded eVLP capsid evolution. FIG. 20A shows a schematic of the barcoded eVLP capsid library generation. Each unique capsid mutant was linked to a unique barcoded sgRNA on the same plasmid vector. These barcoded vectors were used to produce lentivirus, which was then used to generate a barcoded producer cell library in which each producer cell contained a single integration of a barcoded sgRNA and capsid mutant expression cassette. Following expansion of transduced cells, the barcoded producer cell library was transfected with the other plasmids for eVLP production to generate a barcoded eVLP capsid library. FIG. 20B shows a schematic of selections for improved eVLP production and improved eVLP transduction. Barcodes enriched in eVLP-packaged sgRNAs relative to producer cell gDNA identified capsid mutants that support improved eVLP production. Barcodes enriched in eVLP-transduced cells relative to eVLP-packaged sgRNAs identified capsid mutants that support improved eVLP transduction. FIG. 20C shows average barcode enrichment values for each capsid mutant in the production selection and transduction selection. Each capsid mutant is shown as a single dot whose x-coordinate reflects that capsid mutant's average production enrichment and y-coordinate reflects that capsid mutant's average transduction enrichment. The canonical capsid used in v4 eVLPs is shown as a dot labeled “v4,” and the corresponding enrichment values associated with this dot are shown as dotted lines. Capsid mutants selected for further characterization are shown as dots with labels indicating the amino acid substitutions. Production and transduction enrichment values were calculated as the average of n=2 replicates. See also FIGS. 26A-27B.

FIGS. 21A-21C show that evolved capsid mutations improved eVLP potency. FIG. 21A shows fold change in eVLP potency relative to v4 eVLPs of each evolved capsid mutant incorporated individually into the gag-ABE construct and paired with the evolved gag^{Q226P}-pro-pol. FIG. 21B shows fold change in eVLP potency relative to v4 eVLPs of each evolved C-terminal capsid mutant with or without the Q226P mutant incorporated into either the gag-ABE only, gag-pro-pol only, or both gag-ABE and gag-pro-pol. In both FIG. 21A and FIG. 21B, bars reflect the mean of n=3 biological replicates, and dots represent individual replicate values. FIG. 21C shows a comparison of v4 eVLPs and evolved gag-ABE mutants paired with the evolved gag^{Q226P}-pro-pol across a range of eVLP doses. Adenine base editing efficiencies at position A₇ of the BCL11A enhancer site in HEK293T cells are shown. eVLPs were produced at a concentration of approximately 5e8 eVLPs/μL. Dots and error bars represent mean±s.e.m. of n=3 biological replicates. Data were fit to four-parameter logistic curves using nonlinear regression.

FIGS. 22A-22E show characterization of evolved v5 eVLPs. FIG. 22A shows a schematic of the evolved Q226P mutant within the MMLV gag^{Q226P}-pro-pol in v5 eVLPs, located directly downstream of the internal p12/capsid protease cleavage site. FIG. 22B shows a schematic of the evolved C507V mutant within the gag^{C507V}-ABE in v5 eVLPs, located within the CCHC zinc finger motif in the nucleocapsid domain. FIG. 22C shows the percent of cleaved ABE cargo detected in v4 or v5 eVLPs. Bars reflect the mean of n=3 replicates, and dots represent individual replicate values. FIG. 22D shows quantification of ABE molecules per eVLP by anti-Cas9 and anti-MLV (p30) ELISA. FIG. 22E shows fold change in eVLP-packaged sgRNA abundance by RT-qPCR using sgRNA-specific primers, normalized relative to sgRNA abundance in v4 eVLPs. In FIG. 22D and FIG. 22E, bars reflect the mean±s.e.m. of n=3 replicates.

FIGS. 23A-23C show that barcoded sgRNAs are compatible with eVLPs. FIG. 23A shows a schematic of the 15-bp barcode sequence inserted into the tetraloop of the SpCas9 sgRNA scaffold. FIG. 23B shows an overview of the standard eVLP production workflow. FIG. 23C shows adenine base editing efficiencies at position A₇ of the BCL11A enhancer site in cells. eVLPs were produced using a four-plasmid transfection or three-plasmid (combined sgRNA/gag-ABE vector) transfection using either standard or barcoded sgRNAs as shown.

Bars reflect the mean of n=3 biological replicates, and dots represent individual replicate values.

FIGS. 24A-24C show barcoded eVLP capsid library construction. FIG. 24A shows a schematic of the region of the gag that was mutated in the eVLP capsid library. The mutated regions spanned 198 total residues of the capsid and nucleocapsid domains of gag. Each residue was mutated to each of the 19 possible non-wild-type residues at each position, resulting in a total library size of 3,762 single-residue mutants. FIG. 24B shows an overview of the first step of the barcoded capsid library cloning procedure. Each capsid mutant was synthesized with a 15-bp barcode on the same short oligonucleotide and cloned into an appropriate vector backbone by Gibson assembly. FIG. 24C shows an overview of the second step of the barcoded capsid library cloning procedure. The Gibson assembly products from the first cloning step were subjected to a Golden Gate assembly reaction to install the intervening promoters and gene sequences in between the barcoded sgRNA and capsid mutant sequences. These two cloning steps were repeated independently four times to generate four sub-libraries, with each sub-library containing all capsid mutants within a 150 bp region of gag.

FIG. 25 shows barcode frequency distribution in producer-cell gDNA. The theoretical median of this frequency distribution, assuming perfectly equal representation of all library members, is depicted by dotted line B at

$$x = \frac{1}{3763} \approx 0 \text{.000266} .$$

The observed median (0.000225) is depicted by dotted line A.

FIGS. 26A-26B show evolving eVLP capsid mutants with improved production. FIG. 26A shows a schematic of the production selection. The production enrichment value for each barcode was calculated by dividing the frequency of that barcode in the eVLP-packaged sgRNAs by the frequency of that barcode in the producer-cell gDNA. Enriched barcodes identified capsid mutants that support improved production. FIG. 26B shows production enrichment values for the assessed capsid mutant. The capsid mutant is shown as a single dot whose x-coordinate reflects the mutant's enrichment in production selection replicate 1 and y-coordinate reflects the mutant's enrichment in production selection replicate 2. The canonical capsid used in v4 eVLPs is shown as a dot labeled “v4,” and the corresponding enrichment values associated with this dot are shown as dotted lines.

FIGS. 27A-27B show evolving eVLP capsid mutants with improved transduction. FIG. 27A shows a schematic of the transduction selection. The transduction enrichment value for each barcode was calculated by dividing the frequency of that barcode in the successfully delivered sgRNAs by the frequency of that barcode in the eVLP-packaged sgRNAs. Enriched barcodes identified capsid mutants that supported improved transduction. FIG. 27B shows transduction enrichment values for the assessed capsid mutant. The capsid mutant is shown as a single dot whose x-coordinate reflects the mutant's enrichment in transduction selection replicate 1 and y-coordinate reflects the mutant's enrichment in transduction selection replicate 2. The canonical capsid used in v4 eVLPs is shown as a dot labeled “v4,” and the corresponding enrichment values associated with this dot are shown as dotted lines.

FIGS. 28A-28B show incorporating evolved capsid mutants into gag-ABE or gag-pro-pol. FIG. 28A shows fold change in eVLP potency relative to v4 eVLPs of each evolved capsid mutant incorporated individually into the gag-ABE construct and paired with the wild-type MMLV gag-pro-pol. Bars reflect the mean of n=3 biological replicates, and dots represent individual replicate values. FIG. 28B shows a schematic of incorporating different combinations of capsid mutations into either the gag-ABE or gag-pro-pol or both.

FIG. 29 shows an analysis of gag-ABE cleavage in v4 versus v5 eVLPs. Western blot analysis of lysed v4 and v5 eVLPs using an anti-Cas9 antibody is shown. The full-length non-cleaved gag-ABE fusion is ~247 kD and the cleaved ABE is ~184 kD. Three additional cleavage products, which were generated by protease cleavage at one of three cleavage sites internal to gag (see FIG. 24A) and still contained the ABE, were detected at intermediate molecular weights.

FIG. 30 shows v5 eVLP compatibility with barcoded versus standard sgRNAs. Comparison of the potency of v5 eVLPs with barcoded or standard sgRNAs across a range of eVLP doses is shown. Adenine base editing efficiencies at position A₇ of the BCL11A enhancer site in HEK293T cells are shown. eVLPs were produced at a concentration of approximately 5×10⁸ eVLPs/μL. Dots and error bars represent mean±s.e.m. of n=3 biological replicates. Data were fit to four-parameter logistic curves using nonlinear regression.

FIG. 31 shows DLS analysis of purified v4 and v5 eVLPs. Dots and error bars reflect the mean±s.e.m. of n=3 replicates. Smoothing spline curves were generated and fit to the data using GraphPad Prism 10.

FIGS. 32A-32F show that evolved v5 eVLPs outperform previously-disclosed v4 eVLPs at six different genomic loci in mouse N2A cells. Editing at Angptl3 (FIG. 32A), Rosa26 (FIG. 32B), Dnmt1 (FIG. 32C), Pcsk9 Exon 4 (FIG. 32D), Pcsk9 Exon 6 (FIG. 32E), and Pcsk9 Exon 8 (FIG. 32F) is shown.

### Definitions

Unless defined otherwise, all technical and scientific terms used herein have the meaning commonly understood by a person skilled in the art to which this invention belongs.

The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### Base Editors

The term “base editor (BE)” refers to an agent comprising a polypeptide that is capable of making a modification to a base (e.g., A, T, C, G, or U) within a nucleic acid sequence (e.g., DNA or RNA) that converts one base to another (e.g., A to G, A to C, A to T, C to T, C to G, C to A, G to A, G to C, G to T, T to A, T to C, or T to G). In some embodiments, the base editor is capable of deaminating a base within a nucleic acid such as a base within a DNA molecule. In the case of an adenosine base editor, the base editor is capable of deaminating an adenine (A) in DNA. Such base editors may include a nucleic acid programmable DNA binding protein (napDNAbp) fused to an adenosine deaminase. Some base editors include CRISPR-mediated fusion proteins that are utilized in the base editing methods described herein. In some embodiments, the base editor comprises a nuclease-inactive Cas9 (dCas9) fused to a deaminase which binds a nucleic acid in a guide RNA-programmed manner via the formation of an R-loop, but does not cleave the nucleic acid. For example, the dCas9 domain of the fusion protein may include a D10A and a H840A mutation (which renders Cas9 capable of cleaving only one strand of a nucleic acid duplex), as described in International PCT Application, PCT/US2016/058344, filed Oct. 22, 2016, which published as WO 2017/070632 on Apr. 27, 2017, and is incorporated herein by reference. The DNA cleavage domain of S. pyogenes Cas9 includes two subdomains, the HNH nuclease subdomain and the RuvC1 subdomain. The HNH subdomain cleaves the strand complementary to the gRNA (the “targeted strand,” or the strand in which editing or deamination occurs), whereas the RuvC1 subdomain cleaves the non-complementary strand containing the PAM sequence (the “non-edited strand”). The RuvC1 mutant D10A generates a nick in the targeted strand, while the HNH mutant H840A generates a nick on the non-edited strand (see Jinek et al., Science, 337:816-821(2012); Qi et al., Cell. 28; 152(5):1173-83 (2013)). In some embodiments, a base editor comprises a Cas9 nickase (nCas9) that comprises only one of the D10A or the H840A mutations.

In some embodiments, a base editor is a macromolecule or macromolecular complex that results primarily (e.g., more than 80%, more than 85%, more than 90%, more than 95%, more than 99%, more than 99.9%, or 100%) in the conversion of a base in a polynucleotide sequence into another base (i.e., a transition or transversion) using a combination of 1) a nucleotide-, nucleoside-, or base-modifying enzyme and 2) a nucleic acid binding protein that can be programmed to bind to a specific nucleic acid sequence.

In some embodiments, the base editor comprises a DNA binding domain (e.g., a programmable DNA binding domain such as a dCas9 or nCas9) that directs it to a target sequence. In some embodiments, the base editor comprises a base modification domain fused to a programmable DNA binding domain (e.g., dCas9 or nCas9). The terms “base modifying enzyme” and “base modification domain,” which are used interchangeably herein, refer to an enzyme that can modify a base and convert one base to another (e.g., a deaminase such as a cytidine deaminase or an adenosine deaminase). The base modifying enzyme of the base editor may target cytosine (C) bases in a nucleic acid sequence and convert the C to a thymine (T) base. In some embodiments, C to T editing is carried out by a deaminase, e.g., a cytidine deaminase. In some embodiments, A to G editing is carried out by a deaminase, e.g., an adenosine deaminase. Base editors that can carry out other types of base conversions (e.g., C to G) are also contemplated.

In some embodiments, a base editor converts a C to a T. In some embodiments, the base editor comprises a cytosine deaminase. A “cytosine deaminase”, or “cytidine deaminase,” refers to an enzyme that catalyzes the chemical reaction “cytosine+H₂O→uracil+NH₃” or “5-methyl-cytosine+H₂O→thymine+NH₃.” As may be apparent from the reaction formula, such chemical reactions result in a C to U/T base change. In the context of a gene, such a nucleotide change, or mutation, may in turn lead to an amino acid change in the protein, which may affect the protein's function, e.g., loss-of-function or gain-of-function. In some embodiments, the C to T base editor comprises a dCas9 or nCas9 fused to a cytidine deaminase. In some embodiments, the cytidine deaminase domain is fused to the N-terminus of the dCas9 or nCas9. In some embodiments, the base editor further comprises a domain that inhibits uracil glycosylase, and/or a nuclear localization signal. Such base editors have been described in the art, e.g., in Rees & Liu, Nat Rev Genet. 2018; 19(12):770-788 and Koblan et al., Nat Biotechnol. 2018; 36(9):843-846; as well as U.S. Patent Publication No. 2018/0073012, U.S. Patent Publication No. 2017/0121693, PCT Publication No. WO 2017/070633, U.S. Patent Publication No. 2015/0166980, U.S. Pat. Nos. 9,840,699, 10,077,453, PCT Publication No. WO 2019/023680, PCT Publication No. WO 2018/0176009, PCT Publication No. WO2019/226953, PCT Publication No. WO2020/041751, PCT Publication No. WO2020/051360, PCT Publication No. WO2020/214842, PCT Publication No. WO2020/102659, PCT Publication No. WO2020/086908, and PCT Publication No. WO2020/092453, each of which is incorporated herein by reference.

In some embodiments, a base editor converts an A to a G. In some embodiments, the base editor comprises an adenosine deaminase. An “adenosine deaminase” is an enzyme involved in purine metabolism. An adenosine deaminase catalyzes hydrolytic deamination of adenosine (forming inosine, which base pairs as G) in the context of DNA. There are no known natural adenosine deaminases that act on DNA. Instead, known adenosine deaminase enzymes only act on RNA (tRNA or mRNA). Evolved deoxyadenosine deaminase enzymes that accept DNA substrates and deaminate dA to deoxyinosine have been described, e.g., in PCT Application PCT/US2017/045381, filed Aug. 3, 2017, which published as WO 2018/027078, PCT Application No. PCT/US2019/033848, which published as WO 2019/226953, PCT Application No PCT/US2019/033848, filed May 23, 2019, and PCT Patent Application No. PCT/US2020/028568, filed Apr. 17, 2020; each of which is incorporated herein by reference.

Exemplary adenosine and cytidine base editors are also described in Rees & Liu, Base editing: precision chemistry on the genome and transcriptome of living cells, Nat. Rev. Genet. 2018; 19(12):770-788; as well as U.S. Patent Publication No. 2018/0073012, U.S. Patent Publication No. 2017/0121693, PCT Publication No. WO 2017/070633, U.S. Patent Publication No. 2015/0166980, U.S. Pat. Nos. 9,840,699, and 10,077,453, each of which is incorporated herein by reference.

### Cargo

As used herein, the term “cargo” refers to any molecule that is packaged into the VLPs described herein to be delivered to a target cell. Cargo can include protein (including fusion proteins), nucleic acids, and small molecules. In some embodiments, a cargo includes a gene editing agent as described herein. In some embodiments, a cargo includes a gRNA (for example, a barcoded gRNA as described herein. In some embodiments, a cargo comprises or is a napDNAbp. In some embodiments, a cargo is a base editor. In some embodiments, a cargo is a prime editor.

### Cas9

The term “Cas9” or “Cas9 nuclease” refers to an RNA-guided nuclease comprising a Cas9 domain, or a fragment thereof (e.g., a protein comprising an active or inactive DNA cleavage domain of Cas9, and/or the gRNA binding domain of Cas9). A “Cas9 domain,” as used herein, is a protein fragment comprising an active or inactive cleavage domain of Cas9 and/or the gRNA binding domain of Cas9. A “Cas9 protein” is a full length Cas9 protein. A Cas9 nuclease is also referred to sometimes as a casn1 nuclease or a CRISPR (Clustered Regularly Interspaced Short Palindromic Repeat)-associated nuclease. CRISPR is an adaptive immune system that provides protection against mobile genetic elements (viruses, transposable elements, and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc), and a Cas9 domain. The tracrRNA serves as a guide for ribonuclease 3-aided processing of pre-crRNA. Subsequently, Cas9/crRNA/tracrRNA endonucleolytically cleaves a linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), which is incorporated herein by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), each of which is incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; which is incorporated herein by reference. In some embodiments, a Cas9 nuclease comprises one or more mutations that partially impair or inactivate the DNA cleavage domain.

A nuclease-inactivated Cas9 domain may interchangeably be referred to as a “dCas9” protein (for nuclease-“dead” Cas9). Methods for generating a Cas9 domain (or a fragment thereof) having an inactive DNA cleavage domain are known (see, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)). In some embodiments, Cas9 nickases are provided, in which the nuclease activity of only one of the two nuclease domains is inactivated. In certain embodiments, a Cas9 nickase (nCas9) comprises only one of the D10A or the H840A mutations mentioned above. In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, at least about 99.8% identical, or at least about 99.9% identical to a wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9. In some embodiments, the Cas9 variant comprises a fragment of a Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of a wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

### Fusion Protein

The term “fusion protein” as used herein refers to a hybrid polypeptide that comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. Oher examples include fusion of a Cas9 or equivalent thereof to a deaminase (as in a base editor) or to a polymerase such as a reverse transcriptase (as in a prime editor). Any of the fusion proteins provided herein may be produced by any method known in the art. For example, the fusion proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), which is incorporated herein by reference. In some embodiments, a fusion protein is a base editor as described herein. In some embodiments, a fusion protein is a prime editor as described herein. In some embodiments, a fusion protein is another gene editing agent as described herein. In some embodiments, a fusion protein comprises a gag protein, one or more NES, and a cargo as described herein.

### Gene Editing Agent

A “gene editing agent,” as used herein, refers to any agent capable of changing one or more nucleotides in a nucleic acid molecules into one or more other nucleotides, or any agent capable of inserting or deleting one or more nucleotides into or from a nucleic acid molecule. Gene editing agents include, but are not limited to, nucleases such as CRISPR proteins, meganucleases, zinc finger nucleases, and TALENs. In some embodiments, a gene editing agent comprises a napDNAbp as described herein. In some embodiments, a gene editing agent comprises a fusion protein comprising a napDNAbp as described herein. In some embodiments, a gene editing agent is a napDNAbp as described herein. In some embodiments, a gene editing agent comprises a Cas protein as described herein. In some embodiments, a gene editing agent comprises a fusion protein comprising a Cas protein as described herein. In some embodiments, a gene editing agent is a Cas protein as described herein. In some embodiments, a gene editing agent comprises a Cas9 protein as described herein. In some embodiments, a gene editing agent comprises a fusion protein comprising a Cas9 protein as described herein. In some embodiments, a gene editing agent is a Cas9 protein as described herein. In some embodiments, a gene editing agent is a base editor as described herein. In some embodiments, a gene editing agent is a prime editor as described herein. In some embodiments, a gene editing agent comprises a recombinase. In some embodiments, a gene editing agent comprises a deaminase. In some embodiments, a gene editing agent comprises a polymerase. In some embodiments, a gene editing agent comprises a reverse transcriptase. In some embodiments, a gene editing agent comprises an epigenetic modifier.

### Group-Specific Antigen (Gag)

Without being limited by theory, and in the context of typical envelope virus lifecycle, Gag is the primary structural protein responsible for orchestrating the majority of steps in viral assembly, including budding out of fully-formed enveloped virions having an (i) envelope (comprising a lipid membrane formed from cell membrane during budding out, and one or more glycoproteins inserted therein), and (ii) a capsid, which is the internal protein shell. Most of these assembly steps occur via interactions with three Gag subdomains—matrix (MA), capsid (CA), and nucleocapsid. These three regions have a low level of sequence conservation among the different retroviral genera, which belies the observed high level of structural conservation. Outside of these three domains, Gag proteins can vary widely. For example, HIV-1 Gag additionally codes for a C-terminal p6 protein as well as two spacer proteins, SP1 and SP2, which demarcate the CA-NC and NC-p6 junctions, but HTLV-1 contains no additional sequences outside of MA, CA, and NC (Oroszlan and Copeland, Curr. Top. Microbiol. Immunol. 1985, 115, 221-233; Henderson et al., J. Virol. 1992, 66(4), 1856-1865).

Gag is also referred to as a “viral structural protein.” As used herein, the term “viral structural protein” refers to viral proteins that contribute to the overall structure of the capsid protein or of the protein core of a virus. The term “viral structural protein” further includes functional fragments or derivatives of such viral protein contributing to the structure of a capsid protein or of the protein core of a virus. An example of viral structural protein is MMLV Gag. The viral membrane fusion proteins are not considered as viral structural proteins. Typically, said viral structural proteins are localized inside the core of the virus.

In some embodiments, the gag protein used in the VLPs described herein (including the viral nucleocapsid portion) comprises the sequence of an MMLV gag protein of SEQ ID NO: 100, or a sequence at least 70%, at least 75%, at least 80%, at least 85%, at least 90%, at least 95%, at least 96%, at least 97%, at least 98%, or at least 99% identical to the sequence of SEQ ID NO: 100:

(SEQ ID NO: 100)
MGQTVTTPLSLTLGHWKDVERIAHNQSVDVKKRRWVTFCSAEWPT
FNVGWPRDGTFNRDLITQVKIKVFSPGPHGHPDQVPYIVTWEALA
FDPPPWVKPFVHPKPPPPLPPSAPSLPLEPPRSTPPRSSLYPALT
PSLGAKPKPQVLSDSGGPLIDLLTEDPPPYRDPRPPPSDRDGNGG
EATPAGEAPDPSPMASRLRGRREPPVADSTTSQAFPLRAGGNGQL
QYWPFSSSDLYNWKNNNPSFSEDPGKLTALIESVLITHQPTWDDC
QQLLGTLLTGEEKQRVLLEARKAVRGDDGRPTQLPNEVDAAFPLE
RPDWDYTTQAGRNHLVHYRQLLLAGLQNAGRSPTNLAKVKGITQG
PNESPSAFLERLKEAYRRYTPYDPEDPGQETNVSMSFIWQSAPDI
GRKLERLEDLKNKTLGDLVREAEKIFNKRETPEEREERIRRETEE
KEERRRTEDEQKEKERDRRRHREMSKLLATVVSGQKQDRQGGERR
RSQLDRDQCAYCKEKGHWAKDCPKKPRGPRGPRPQTSLLTLDD.

### Group-Specific Antigen (Gag) Nucleocapsid Protein

The term “group-specific antigen nucleocapsid protein” or “gag nucleocapsid protein” refers to a protein that makes up the core structural component of the inner shell of many viruses, including retroviruses. The gag nucleocapsid proteins used in the VLPs of the present disclosure may be an MMLV gag nucleocapsid protein, an FMLV gag nucleocapsid protein, or a nucleocapsid protein from any other virus that produces such proteins.

### Group-Specific Antigen (Gag) Protease (Pro) Polyprotein

A “group-specific antigen (gag) protease (pro) polyprotein” or “gag-pro polyprotein” refers to a gag nucleocapsid protein further comprising a viral protease linked thereto. Gag-pro polyproteins mediate proteolytic cleavage of gag and gag-pol polyproteins or nucleocapsid proteins during or shortly after the release of a virion from the plasma membrane. In the VLPs described herein, the protease of a gag-pro polyprotein is responsible for cleaving a cleavable linker in the fusion protein to release a cargo (e.g., a cargo protein such as a base editor or prime editor) following delivery of the VLP to a target cell. In some embodiments, a gag-pro polyprotein is an MMLV gag-pro polyprotein or an FMLV gag-pro polyprotein.

Guide RNA (“gRNA”)

As used herein, the term “guide RNA” is a particular type of guide nucleic acid which is mostly commonly associated with a Cas protein of a CRISPR-Cas9 and which associates with Cas9, directing the Cas9 protein to a specific sequence in a DNA molecule that includes complementarity to the protospacer sequence of the guide RNA. However, this term also embraces the equivalent guide nucleic acid molecules that associate with Cas9 equivalents, homologs, orthologs, or paralogs, whether naturally occurring or non-naturally occurring (e.g., engineered or recombinant), and which otherwise program the Cas9 equivalent to localize to a specific target nucleotide sequence. The Cas9 equivalents may include other napDNAbp from any type of CRISPR system (e.g., type II, V, VI), including Cpf1 (a type-V CRISPR-Cas system), C2c1 (a type V CRISPR-Cas system), C2c2 (a type VI CRISPR-Cas system), and C2c3 (a type V CRISPR-Cas system). Further Cas-equivalents are described in Makarova et al., “C2c2 is a single-component programmable RNA-guided RNA-targeting CRISPR effector,” Science 2016; 353(6299), which is incorporated herein by reference.

A guide RNA is a particular type of guide nucleic acid that is most commonly associated with a Cas protein of a CRISPR-Cas9 and which associates with Cas9, directing the Cas9 protein to a specific sequence in a DNA molecule that includes complementarity to the protospacer sequence for the guide RNA. Functionally, guide RNAs associate with Cas9, directing (or programming) the Cas9 protein to a specific sequence in a DNA molecule that includes a sequence complementary to the protospacer sequence for the guide RNA. A gRNA is a component of the CRISPR/Cas system. Typically, a guide RNA comprises a fusion of a CRISPR-targeting RNA (crRNA) and a trans-activation crRNA (tracrRNA), providing both targeting specificity and scaffolding/binding ability for Cas9 nuclease. A “crRNA” is a bacterial RNA that confers target specificity and requires tracrRNA to bind to Cas9. A “tracrRNA” is a bacterial RNA that links the crRNA to the Cas9 nuclease and typically can bind any crRNA. The sequence specificity of a Cas DNA-binding protein is determined by gRNAs, which have nucleotide base-pairing complementarity to target DNA sequences. The native gRNA comprises a 20 nucleotide (nt) Specificity Determining Sequence (SDS), or spacer, which specifies the DNA sequence to be targeted, and is immediately followed by an 80 nt scaffold sequence, which associates the gRNA with Cas9. In some embodiments, an SDS of the present disclosure has a length of 15 to 100 nucleotides, or more. For example, an SDS may have a length of 15 to 90, 15 to 85, 15 to 80, 15 to 75, 15 to 70, 15 to 65, 15 to 60, 15 to 55, 15 to 50, 15 to 45, 15 to 40, 15 to 35, 15 to 30, or 15 to 20 nucleotides. In some embodiments, the SDS is 20 nucleotides long. For example, the SDS may be 15, 16, 17, 18, 19, 20, 21, 22, 23, 24, or 25 nucleotides long. At least a portion of the target DNA sequence is complementary to the SDS of the gRNA. For Cas9 to successfully bind to the DNA target sequence, a region of the target sequence is complementary to the SDS of the gRNA sequence and is immediately followed by the correct protospacer adjacent motif (PAM) sequence (e.g., NGG for Cas9; and TTN, TTTN, or YTN for Cpf1). In some embodiments, an SDS is 100% complementary to its target sequence. In some embodiments, the SDS sequence is less than 100% complementary to its target sequence and is, thus, considered to be partially complementary to its target sequence. For example, a targeting sequence may be 99%, 98%, 97%, 96%, 95%, 94%, 93%, 92%, 91%, or 90% complementary to its target sequence. In some embodiments, the SDS of template DNA or target DNA may differ from a complementary region of a gRNA by 1, 2, 3, 4, or 5 nucleotides.

In some embodiments, the guide RNA is about 15-120 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 97, 98, 99, 100, 101, 102, 103, 104, 105, 106, 107, 108, 109, 110, 111, 112, 113, 114, 115, 116, 117, 118, 119, or 120 nucleotides long. In some embodiments, the guide RNA comprises a sequence of 10, 11, 12, 13, 14, 15, 16, 17, 18, 19, 20, or more contiguous nucleotides that is complementary to a target sequence. Sequence complementarity refers to distinct interactions between adenine and thymine (DNA) or uracil (RNA), and between guanine and cytosine.

In some embodiments, a guide RNA comprises a nucleic acid barcode sequence, for example, a barcode sequence associated with a particular variant of a component of a VLP as described herein. In some embodiments, a barcode sequence on a gRNA is associated with a particular variant of a viral nucleocapsid protein in a VLP. Such barcodes can be packaged into a VLP as it is produced and then sequenced and used, for example, to determine which VLPs comprise variants of a nucleocapsid protein or other component that confer one or more improved properties relative to a VLP that comprises a wild type nucleocapsid protein or other component of the VLP.

In some embodiments, a barcode sequence is inserted in a portion of a gRNA outside of the Cas9-gRNA ribonucleoprotein complex (i.e., in a portion of the gRNA to which Cas9 does not bind). In some embodiments, a barcode sequence is inserted into the P1 stem/tetraloop portion of a gRNA. In some embodiments, a barcode sequence is 5-25, 6-24, 7-23, 8-22, 9-21, 10-20, 11-19, 12-18, 13-17, or 14-16 nucleotides long. In some embodiments, a barcode sequence is 5, 6, 7, 8, 9, 10, 11, 12, 13, 14, 15, 16, 17, 18, 19, 20, 21, 22, 23, 24, or 25 nucleotides long. In certain embodiments, a barcode sequence is 15 nucleotides long. Such gRNAs comprising a barcode sequence may be included on a plasmid or other vector along with a variant of a VLP component (such as a nucleocapsid protein variant) in the libraries provided herein.

In some embodiments, a guide RNA is a prime editing guide RNA (PEgRNA). PEgRNAs may also comprise barcode sequences and be packaged into VLPs as described herein. As used herein, the terms “prime editing guide RNA” or “PEgRNA” or “extended guide RNA” refer to a specialized form of a guide RNA that has been modified to include one or more additional sequences for implementing the prime editing methods and compositions described herein. As described herein, the prime editing guide RNAs comprise one or more “extended regions” of nucleic acid sequence. The extended regions may comprise, but are not limited to, single-stranded RNA or DNA. Further, the extended regions may occur at the 3′ end of a traditional guide RNA. In other arrangements, the extended regions may occur at the 5′ end of a traditional guide RNA. In still other arrangements, the extended region may occur at an intramolecular region of the traditional guide RNA, for example, in the gRNA core region which associates and/or binds to the napDNAbp. The extended region comprises a “DNA synthesis template” which encodes (by the polymerase of the prime editor) a single-stranded DNA which, in turn, has been designed to be (a) homologous with the endogenous target DNA to be edited, and (b) which comprises at least one desired nucleotide change (e.g., a transition, a transversion, a deletion, or an insertion) to be introduced or integrated into the endogenous target DNA. The extended region may also comprise other functional sequence elements, such as, but not limited to, a “primer binding site” and a “spacer or linker” sequence, or other structural elements, such as, but not limited to aptamers, stem loops, hairpins, toe loops (e.g., a 3′ toeloop), or an RNA-protein recruitment domain (e.g., MS2 hairpin). As used herein, the “primer binding site” comprises a sequence that hybridizes to a single-strand DNA sequence having a 3′end generated from the nicked DNA of the R-loop.

In certain embodiments, the PEgRNAs have a 5′ extension arm, a spacer, and a gRNA core. The 5′ extension further comprises in the 5′ to 3′ direction a reverse transcriptase template, a primer binding site, and a linker. The reverse transcriptase template may also be referred to more broadly as the “DNA synthesis template” where the polymerase of a prime editor described herein is not an RT, but another type of polymerase.

In certain other embodiments, the PEgRNAs have a 5′ extension arm, a spacer, and a gRNA core. The 5′ extension further comprises in the 5′ to 3′ direction a reverse transcriptase template, a primer binding site, and a linker. The reverse transcriptase template may also be referred to more broadly as the “DNA synthesis template” where the polymerase of a prime editor described herein is not an RT, but another type of polymerase.

In still other embodiments, the PEgRNAs have in the 5′ to 3′ direction a spacer (1), a gRNA core (2), and an extension arm (3). The extension arm (3) is at the 3′ end of the PEgRNA. The extension arm (3) further comprises in the 5′ to 3′ direction a “primer binding site” (A), an “edit template” (B), and a “homology arm” (C). The extension arm (3) may also comprise an optional modifier region at the 3′ and 5′ ends, which may be the same sequences or different sequences. In addition, the 3′ end of the PEgRNA may comprise a transcriptional terminator sequence. These sequence elements of the PEgRNAs are further described and defined herein.

In still other embodiments, the PEgRNAs have in the 5′ to 3′ direction an extension arm (3), a spacer (1), and a gRNA core (2). The extension arm (3) is at the 5′ end of the PEgRNA. The extension arm (3) further comprises in the 3′ to 5′ direction a “primer binding site” (A), an “edit template” (B), and a “homology arm” (C). The extension arm (3) may also comprise an optional modifier region at the 3′ and 5′ ends, which may be the same sequences or different sequences. The PEgRNAs may also comprise a transcriptional terminator sequence at the 3′ end. These sequence elements of the PEgRNAs are further described and defined herein.

### Linkers

The term “linker,” as used herein, refers to a molecule linking two other molecules or moieties. The linker can be an amino acid sequence in the case of a linker joining two fusion proteins. For example, a Cas9 can be fused to a deaminase (e.g., an adenosine deaminase or a cytosine deaminase) by an amino acid linker sequence. A Cas9 can be fused to a reverse transcriptase by an amino acid linker sequence. The linker can also be a nucleotide sequence in the case of joining two nucleotide sequences together (e.g., in a gRNA). In other embodiments, the linker is an organic molecule, group, polymer, or chemical moiety. In some embodiments, the linker is 5-200 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

A “cleavable linker” refers to a linker that can be split or cut by any means. The linker can be an amino acid sequence. In some embodiments, the linker between the NES and the napDNAbp of the VLPs described herein comprises a cleavable linker. A cleavable linker may comprise a self-cleaving peptide (e.g., a 2A peptide such as EGRGSLLTCGDVEENPGP (SEQ ID NO: 9), ATNFSLLKQAGDVEENPGP (SEQ ID NO: 10), QCTNYALLKLAGDVESNPGP (SEQ ID NO: 11), or VKQTLNFDLLKLAGDVESNPGP (SEQ ID NO: 12)). In some embodiments, a cleavable linker comprises a protease cleavage site that is cut after being contacted by a protease. For example, the present disclosure contemplates that use of cleavable linkers comprising a protease cleavage site of amino acid sequences TSTLLMENSS (SEQ ID NO: 1), PRSSLYPALTP (SEQ ID NO: 2), VQALVLTQ (SEQ ID NO: 3), PLQVLTLNIERR (SEQ ID NO: 4), or an amino acid sequence at least 90% identical to any one of SEQ ID NOs: 1-4. In certain embodiments, a cleavable linker comprises an MMLV protease cleavage site of an FMLV protease cleavage site.

The protease cleavage site may be any known in the art, or any sequence yet to be discovered, so long as the corresponding protease may be co-packaged in the VLPs to allow for post-maturation cleavage within the mature VLP particles. Such cleavage sites and their corresponding proteases include, but are not limited to: (a) granzyme A, which recognizes and cleaves a sequence comprising ASPRAGGK (SEQ ID NO: 5), (b) granzyme B, which recognizes and cleaves a sequence comprising YEADSLEE (SEQ ID NO: 6), (c) granzyme K, which recognizes and cleaves a sequence comprising YQYRAL (SEQ ID NO: 7), and (d) Cathepsin D, which recognizes and cleaves a sequence comprising LGVLIV (SEQ ID NO: 8). Many other combinations of specific proteases and protease cleavage sites may be used in connection with the present disclosure by co-packing a specific protease during the VLP manufacture process. Such proteases can include, without limitation, Arg-C proteinase, Asp-N Endopeptidase, Caspase 1, Caspase 2, Caspase 3, Caspase 4, Caspase 5, Caspase 7, Caspase 8, Caspase 9, Caspase 10, Chymotrypsin, Clostripain, Enterokinase, Factor Xa, Glutamyl endopeptidase, Granzyme B, Neutrophil elastase, Pepsin, Prolyl-endopeptidase, Proteinase K, Staphylococcal peptidase I, Thermolysin, Thrombin, and Trypsin. Any protease paired with its cognate recognition sequence may be used in the present disclosure protease-sensitive linkers, including any serine protease, cysteine protease, aspartic protease, threonine protease, glutamic protease, metalloprotease, or asparagine peptide lyase (which constitute major classifications of known proteases). The specific protease cleavage sites for said enzymes are well-known in the art and may be utilized in the linkers herein to provide protease-susceptible linkers.

napDNAbp

As used herein, the term “nucleic acid programmable DNA binding protein” or “napDNAbp,” of which Cas9 is an example, refers to a protein that uses RNA:DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9 or equivalent) to localize and bind to a complementary sequence.

Without being bound by theory, the binding mechanism of a napDNAbp - guide RNA complex, in general, includes the step of forming an R-loop whereby the napDNAbp induces the unwinding of a double-strand DNA target, thereby separating the strands in the region bound by the napDNAbp. The guide RNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA, leaving various types of lesions. For example, the napDNAbp may comprise a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”). Exemplary sequences for these and other napDNAbp are provided herein.

### Nuclear Export Sequence (NES)

The term “nuclear export sequence” or “NES” refers to an amino acid sequence that promotes transport of a protein out of the nucleus of the cell to the cytoplasm, for example, through the nuclear pore complex by nuclear transport. Nuclear export sequences are known in the art and would be apparent to the skilled artisan. For example, NES sequences are described in Xu, D. et al. Sequence and structural analyses of nuclear export signals in the NESdb database. Mol Biol. Cell. 2012, 23(18) 3677-3693, the contents of which are incorporated herein by reference. Exemplary NES include, but are not limited to, the following:

| Sequence | SEQ ID NO |
|---|---|
| MEELSQALASSFSV | 22 |
| PLQLPPLERLTL | 23 |
| NELALKLAGLDI | 24 |
| ERFEMFRELNEALEL | 25 |
| DHAEKVAEKLEALSV | 26 |
| QLVEELLKIICAFQL | 27 |
| TNLEALQKKLEELEL | 28 |
| DVKEEMTSALATMRV | 29 |
| STNGSLAAEFRHLQL | 30 |
| PSVQELTEQIHRLLM | 31 |
| MNFKELKDFLKELNI | 32 |
| ENFEILMKLKESLEL | 33 |
| FETVYELTKMCTIR | 34 |
| SGKASSSLGLQDFDL | 35 |
| PKYSDIDVDGLCSEL | 36 |
| VDLACTPTDVRDVDI | 37 |
| YGEKTTQRDLTELEI | 38 |
| RRIYDITNVLEGIGL | 39 |
| AKIIPYSGLLLVITV | 40 |
| LRSEEVHWLHVDMGV | 41 |
| LQSEEVHWLHLDMGV | 42 |
| LQVRKYSLDLASLIL | 43 |
| AGVEAIIRILQQLLF | 44 |
| TGVEALIRILQQLLF | 45 |
| IVLNQLCVRFFGLDL | 46 |
| SLGGFEITPPVVLRL | 47 |
| EAIQDLCLAVEEVSL | 48 |
| DELLQVLRMMVGVNI | 49 |
| SVMLAVQEGIDLLTF | 50 |
| LSSHFQELSI | 51 |
| QSTHVDIRTLEDLLM | 52 |
| ESSAEDLRTLQQLFL | 53 |
| EFSLPTHHTVRLIRV | 54 |
| MSSGYYLGEILRLAL | 55 |
| DTVLDILRDFFELRL | 56 |
| NSVNEILSEFYYVRL | 57 |
| CAFLSVKKQFEELTL | 58 |
| ISPEHVIQALESLGF | 59 |
| AHWMRQLVSFQKLKL | 60 |
| ATRELDELMASLSDF | 61 |
| YQNIELITFINALKL | 62 |
| FNATAVVRHMRKLQL | 63 |
| SGIFGLVTNLEELEV | 64 |
| EESYTLNSDLARLGV | 65 |
| EESYDLTSHLARLGV | 66 |
| GIQQAHAEQLANMRI | 67 |
| DVKEEMTSALATMRV | 29 |
| AAEPVILDLRDLFQL | 68 |
| MEGCVSNLMV | 69 |
| EGCVSNLMV | 70 |
| DMDFLRNLFSQTLSL | 71 |
| EQLLEIVHDLENLSL | 72 |
| NVMKYFTDLFDYLPL | 73 |
| KVYPIILRLGSNLSL | 74 |
| YAGFSLPHAILRIDL | 75 |
| EIVRDIKEKLCYVAL | 76 |
| EAINKLESNLRELQI | 77 |
| EAINKLENNLRELQI | 78 |
| SDQKQEQLLLKKMYL | 79 |
| KQVLWDRTFSLFQQL | 80 |
| AQLQNLTKRIDSLPL | 81 |
| NDENEHQLSLRTVSL | 82 |
| ISFTEFVKVLEKVDV | 83 |
| MESAITLWQFLLQL | 84 |
| VPKELMQQIENFEKI | 85 |
| QARFILEKIDGKIII | 86 |
| QVKFIKMIIEKELTV | 87 |
| NHRMKNLREISQLGI | 88 |
| NHRVKKLNEISKLGI | 89 |
| TEKHLQKYLRQDLRL | 90 |
| RQERKRPLLDLHIEL | 91 |
| ANMRIQDLKVSLKPL | 92 |
| ATMRVDYEQIKIKKI | 93 |
| LQGEEFVCLKSIILL | 94 |
| THYGQKAILFLPLPV | 95 |
| PSAHEITGLADSLQL | 96 |
| VRLHDVLHSDKKLTL | 97 |
| LINRNGELKLANFGL | 98 |
| LEPLKKLECLKSLDL | 99 |

The NES examples above are not limiting. The fusion proteins delivered by the presently described VLPs may comprise any known NES sequence, including any of those described in Xu, D. et al., Sequence and structural analyses of nuclear export signals in the NESdb database. Mol. Biol. Cell. 2012, 23(18), 3677-3693; Fung, H. Y. J. et al., Structural determinants of nuclear export signal orientation in binding to exportin CRM1. eLife. 2015, 4:e10034; and Kosugi, S. et al., Nuclear Export Signal Consensus Sequences Defined Using a Localization-based Yeast Selection System. Traffic. 2008, 9(12), 2053-2062, each of which is incorporated herein by reference.

In various embodiments, the fusion proteins, constructs encoding the fusion proteins, and VLPs disclosed herein further comprise one or more, preferably, at least three nuclear export sequences. In certain embodiments, the fusion proteins comprise at least three NESs. In embodiments with at least three NESs, the NESs can be the same NESs, or they can be different NESs. In certain other embodiments, the fusion proteins, constructs encoding the fusion proteins, and VLPs may comprise at least one, at least two, at least three, at least four, at least five, at least six, at least seven, at least eight, at least nine, at least ten, or more NES. In certain embodiments, the VLPs comprise three NES. In general, the one or more NESs are of sufficient strength to drive accumulation of the VLP proteins (e.g., the Gag-cargo) in a detectable amount in the cytoplasm of a producer cell.

The location of the NES fusion can be at the N-terminus or the C-terminus, or within a sequence of a fusion protein (e.g., inserted between the cargo component and the gag nucleocapsid protein). In certain preferred embodiments, the NES (or multiple NESs, e.g., three NESs) are positioned between the napDNAbp and the gag nucleocapsid protein such that they can be cleaved from the napDNAbp upon delivery of the fusion protein to a target cell. NES sequences may preferably be joined to a fusion protein via a cleavable linker, such as protease-cleavable linker (e.g., the Gag-Pro-Pol). In this way, the NES may be removed from the cargo after VLP maturation so that the cargo may be free to translocate to the nucleus once delivered to a target cell.

The NESs may be any known NES in the art. The NES may also be any NES for nuclear export discovered in the future. The NESs also may be any naturally-occurring NES, or any non-naturally occurring NES (e.g., an NES with one or more desired mutations). In some embodiments, the NES is any of those provided in the table above and comprising one or more, two or more, of three or more mutations.

In one aspect of the disclosure, a base editor or other fusion protein may be modified with one or more nuclear export sequences (NES), preferably at least three NESs. In certain embodiments, the fusion proteins are modified with two or more, three or more, four or more, five or more, six or more, seven or more, eight or more, nine or more, or ten or more NESs. The disclosure contemplates the use of any nuclear export sequence known in the art at the time of the disclosure, or any nuclear export sequence that is identified or otherwise made available in the art after the time of the instant filing. A representative nuclear export sequence is a peptide sequence that directs the protein out of the nucleus of the cell in which the sequence is expressed. NESs commonly contain hydrophobic amino acid residues in the sequence LXXXLXXLXL, where L is a hydrophobic residue (frequently leucine), and X represents any amino acid. Nuclear export sequences often comprise leucine residues.

The fusion proteins delivered by the VLPs described herein may also comprise nuclear export sequences that are linked through one or more linkers, e.g., a polymeric, amino acid, nucleic acid, polysaccharide, or chemical linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and can be joined to cargo (e.g., a base editor or prime editor) by any suitable strategy that effectuates forming a bond (e.g., covalent linkage, hydrogen bonding) between the cargo and the one or more NESs. In some embodiments, the linker joining one or more NES and a cargo is a cleavable linker, as described further herein, so that the one or more NES can be cleaved from the cargo, e.g., upon delivery of the cargo to a target cell.

### Nuclear Localization Sequence (NLS)

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT Application, PCT/EP2000/011690, filed Nov. 23, 2000, published as WO 2001/038547 on May 31, 2001, the contents of which are incorporated herein by reference for its disclosure of exemplary nuclear localization sequences. In some embodiments, an NLS comprises the amino acid sequence PKKKRKV (SEQ ID NO: 13), MDSLLMNRRKFLYQFKNVRWAKGRRETYLC (SEQ ID NO: 14), KRTADGSEFESPKKKRKV (SEQ ID NO: 16), KRTADGSEFEPKKKRKV (SEQ ID NO: 17), NLSKRPAAIKKAGQAKKKK (SEQ ID NO: 18), PAAKRVKLD (SEQ ID NO: 15), RQRRNELKRSF (SEQ ID NO: 19), or NQSSNFGPMKGGNFGGRSSGPYGGGGQYFAKPRNQGGY (SEQ ID NO: 20).

In one aspect of the disclosure, a cargo such as a base editor, prime editor, or other fusion protein may be modified with one or more nuclear localization sequences (NLS), preferably at least two NLSs. In certain embodiments, the cargo is modified with two or more NLSs. The disclosure contemplates the use of any nuclear localization sequence known in the art at the time of the disclosure, or any nuclear localization sequence that is identified or otherwise made available in the art after the time of the instant filing. A representative nuclear localization sequence is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization sequences often comprise proline residues. A variety of nuclear localization sequences have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett. 461:229-34, which is incorporated herein by reference. Translocation is currently thought to involve nuclear pore proteins.

Most NLSs can be classified in three general groups: (i) a monopartite NLS exemplified by the SV40 large T antigen NLS (PKKKRKV (SEQ ID NO: 13)); (ii) a bipartite motif consisting of two basic domains separated by a variable number of spacer amino acids and exemplified by the Xenopus nucleoplasmin NLS (KRXXXXXXXXXXKKKL (SEQ ID NO: 21)); and (iii) noncanonical sequences such as M9 of the hnRNP Al protein, the influenza virus nucleoprotein NLS, and the yeast Gal4 protein NLS (Dingwall and Laskey 1991).

Nuclear localization sequences appear at various points in the amino acid sequences of proteins. NLS have been identified at the N-terminus, the C-terminus, and in the central region of proteins. Thus, the disclosure provides fusion proteins that may be modified with one or more NLSs at the C-terminus and/or the N-terminus, as well as at internal regions of the fusion protein. The residues of a longer sequence that do not function as component NLS residues should be selected so as not to interfere, for example, tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a fusion protein to include one or more NLSs. In one aspect, the fusion proteins may be engineered to express a fusion protein that is translationally fused at its N-terminus or its C-terminus (or both) to one or more NLSs, i.e., to form a base editor or prime editor-NLS fusion construct. In other embodiments, a fusion protein-encoding nucleotide sequence may be genetically modified to incorporate a reading frame that encodes one or more NLSs in an internal region of the encoded base editor or prime editor. In addition, the NLSs may include various amino acid linkers or spacer regions encoded between the base editor and the N-terminally, C-terminally, or internally-attached NLS amino acid sequence, e.g., and in the central region of proteins. Thus, the present disclosure also provides for nucleotide constructs, vectors, and host cells for expressing fusion proteins that comprise a base editor or prime editor and one or more NLSs, among other components.

The fusion proteins delivered by the VLPs described herein may also comprise nuclear localization sequences that are linked to the fusion protein through one or more linkers, e.g., a polymeric, amino acid, nucleic acid, polysaccharide, or chemical linker element. The linkers within the contemplated scope of the disclosure are not intended to have any limitations and can be any suitable type of molecule (e.g., polymer, amino acid, polysaccharide, nucleic acid, lipid, or any synthetic chemical linker domain) and can be joined to the fusion protein by any suitable strategy that effectuates forming a bond (e.g., covalent linkage) between the fusion protein and the one or more NLSs.

### Nucleic Acid Molecule

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5 bromouridine, C5 fluorouridine, C5 iodouridine, C5 propynyl uridine, C5 propynyl cytidine, C5 methylcytidine, 7 deazaadenosine, 7 deazaguanosine, 8 oxoadenosine, 8 oxoguanosine, O(6) methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′ N phosphoramidite linkages).

### Nucleocapsid Protein

The term “nucleocapsid protein” refers to a protein that makes up the core structural component of the inner shell of many viruses, including retroviruses. A nucleocapsid protein is typically part of the gag protein. The nucleocapsid proteins used in the VLPs of the present disclosure may be an MMLV gag nucleocapsid protein, an FMLV gag nucleocapsid protein, or a nucleocapsid protein from any other virus that produces such proteins.

### Prime Editor

The term “prime editor” refers to fusion constructs comprising a napDNAbp (e.g., Cas9 nickase) and a reverse transcriptase that are capable of carrying out prime editing on a target nucleotide sequence in the presence of a PEgRNA (or “extended guide RNA”). The term “prime editor” may refer to the fusion protein or to the fusion protein complexed with a PEgRNA, and/or further complexed with a second-strand nicking sgRNA. In some embodiments, the prime editor may also refer to the complex comprising a fusion protein (reverse transcriptase fused to a napDNAbp) and a PEgRNA.

Prime editors may be used to carry out prime editing, which is an approach for gene editing using napDNAbps, a polymerase (e.g., a reverse transcriptase), and specialized guide RNAs that include a primer binding site and a DNA synthesis template for encoding desired new genetic information (or deleting genetic information) that is then incorporated into a target DNA sequence. Prime editing is described in Anzalone, A. V. et al., Search-and-replace genome editing without double-strand breaks or donor DNA. Nature 576, 149-157 (2019), which is incorporated herein by reference. See also International PCT Application, PCT/US2020/023721, filed Mar. 19, 2020, and published as WO 2020/191239, which is incorporated herein by reference.

Prime editing represents a platform for genome editing that is a versatile and precise method to directly write new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand (or is homologous to it) immediately downstream of the nick site of the target site to be edited (with the exception that it includes the desired edit). Through DNA repair and/or replication machinery, the endogenous strand downstream of the nick site is replaced by the newly synthesized replacement strand containing the desired edit. Cas protein-reverse transcriptase fusions or related systems are used to target a specific DNA sequence with a guide RNA, generate a single strand nick at the target site, and use the nicked DNA as a primer for reverse transcription of an engineered DNA synthesis template that is integrated with the guide RNA. However, while the concept begins with prime editors that use reverse transcriptase as the DNA polymerase component, the prime editors described herein are not limited to reverse transcriptases but may include the use of virtually any DNA polymerase. Indeed, while the application throughout may refer to prime editors with “reverse transcriptases,” it is set forth here that reverse transcriptases are only one type of DNA polymerase that may work with prime editing. Thus, wherever the specification mentions a “reverse transcriptase,” the person having ordinary skill in the art should appreciate that any suitable DNA polymerase may be used in place of the reverse transcriptase. Thus, in one aspect, the prime editors may comprise Cas9 (or an equivalent napDNAbp), which is programmed to target a DNA sequence by associating it with a specialized guide RNA (i.e., PEgRNA) containing a spacer sequence that anneals to a complementary sequence (the complementary sequence to an endogenous protospacer sequence) in the target DNA. The PEgRNA also contains new genetic information in the form of an extension that encodes a replacement strand of DNA containing a desired nucleotide change which is used to replace a corresponding endogenous DNA strand at the target site. To transfer information from the PEgRNA to the target DNA, the mechanism of prime editing involves nicking the target site in one strand of the DNA to expose a 3′-hydroxyl group. The exposed 3′-hydroxyl group can then be used to prime the DNA polymerization of the edit-encoding extension on PEgRNA directly into the target site. In various embodiments, the extension—which provides the template for polymerization of the replacement strand containing the edit—can be formed from RNA or DNA. In the case of an RNA extension, the polymerase of the prime editor can be an RNA-dependent DNA polymerase (such as a reverse transcriptase). In the case of a DNA extension, the polymerase of the prime editor may be a DNA-dependent DNA polymerase. The newly synthesized strand (i.e., the replacement DNA strand containing the desired nucleotide edit) that is formed by the prime editor would be homologous to the genomic target sequence (i.e., have the same sequence as), except for the inclusion of one or more desired nucleotide changes (e.g., a single nucleotide substitution, a deletion, or an insertion, or a combination thereof). The newly synthesized (or replacement) strand of DNA may also be referred to as a single strand DNA flap, which would compete for hybridization with the complementary homologous endogenous DNA strand, thereby displacing the corresponding endogenous strand. Resolution of the hybridized intermediate (also referred to as a heteroduplex, comprising the single strand DNA flap synthesized by the reverse transcriptase hybridized to the endogenous DNA strand with the exception of mismatches at positions where desired nucleotide edits are installed in the edit strand) can include removal of the resulting displaced flap of endogenous DNA (e.g., with a 5′ end DNA flap endonuclease, FEN1), ligation of the synthesized single strand DNA flap to the target DNA, and assimilation of the desired nucleotide changes as a result of cellular DNA repair and/or replication processes.

### Producer Cell

The term “producer cell” refers to any cell type that can be used to make the VLPs described herein. One or more polynucleotides encoding the components of a VLP are transfected, transduced, electroporated, or otherwise inserted into a producer cell. In some embodiments, a single vector comprises polynucleotides encoding all components of the VLP, and in other embodiments, polynucleotides encoding each component of the VLP are split over two, three, or four different vectors. Once the producer cell expresses the polynucleotides, the various components of the VLPs self-assemble spontaneously within the producer cell. Assembly of the VLPs relies on multimerization of the gag polyproteins encoded on the polynucleotides as described above. The gag polyproteins (some of which are fused to a cargo molecule, such as a protein) multimerize at the cell membrane of a producer cell and are subsequently released into the producer cell supernatant spontaneously. In some embodiments, a producer cell is a human cell. In some embodiments, a producer cell is a mammalian cell. Producer cell lines include, but are not limited to, Gesicle 293T cells.

### Protease Cleavage Site

The term “protease cleavage site,” as used herein, refers to an amino acid sequence that is recognized and cleaved by a protease, i.e., an enzyme that catalyzes proteolysis and breaks down proteins into smaller polypeptides, or single amino acids. In some embodiments, a protease cleavage site is included in a cleavable linker in a fusion protein, as described herein. In certain embodiments, a protease cleavage site is cleaved by the protease of a gag-pro polyprotein. In some embodiments, a protease cleavage site comprises an MMLV protease cleavage site or an FMLV protease cleavage site. In certain embodiments, a protease cleavage site comprises one of the amino acid sequences TSTLLMENSS (SEQ ID NO: 1), PRSSLYPALTP (SEQ ID NO: 2), VQALVLTQ (SEQ ID NO: 3), PLQVLTLNIERR (SEQ ID NO: 4), or an amino acid sequence at least 90% identical to any one of SEQ ID NOs: 1-4. In some embodiments, a protease cleavage site comprises an amino acid sequence of any one of SEQ ID NOs: 1-8, or an amino acid sequence at least 90% identical to any one of SEQ ID NOs: 1-8.

### Protein, Peptide, and Polypeptide

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the contents of which are incorporated herein by reference.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cow, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

### Substitution

The term “substitution,” as used herein, refers to replacement of a residue within a sequence, e.g., a nucleic acid or amino acid sequence, with another residue, or a deletion or insertion of one or more residues within a sequence. The term “mutation” may also be used throughout the present disclosure to refer to a substitution (i.e., a “nucleic acid mutation” or an “amino acid mutation”). Substitutions are typically described herein by identifying the original residue followed by the position of the residue within the sequence and the identity of the newly mutated/substituted residue. Various methods for making the amino acid substitutions provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). In some embodiments, a substitution is in a viral nucleocapsid protein, which may be part of a VLP as described herein.

### Target Cell

A “target cell” refers to any cell type to which a VLP is delivered. In some embodiments, a VLP is used to deliver a particular cargo to a target cell. For example, a VLP may be used to deliver a cargo (e.g., a gene editing agent such as a napDNAbp (e.g., a Cas9 protein), a base editor, or a prime editor) to a target cell. Once the VLP enters the target cell, the cargo is released and may perform its function, for example, by editing the genome of the target cell. Target cell types include any cell to which a person of ordinary skill in the art may want to deliver a VLP as described herein. In some embodiments, VLPs are delivered into human embryonic kidney (HEK) cells (e.g., HEK 293 or HEK 293T cells). In some embodiments, VLPs are delivered into stem cells (e.g., human stem cells) such as, for example, pluripotent stem cells (e.g., human pluripotent stem cells, including human induced pluripotent stem cells (hiPSCs)).

Target cell types contemplated by the present disclosure include, but are not limited to, stem and progenitor cells (e.g., embryonic stem cells, hematopoietic stem cells, mesenchymal stem cells, neural crest cells, etc.), endothelial cells, muscle cells, myocardial cells, smooth and skeletal muscle cells, mesenchymal cells, epithelial cells, hematopoietic cells, lymphocytes such as T-cells (e.g., Thl T cells, Th2 T cells, ThO T cells, cytotoxic T cells) and B cells (e.g., pre-B cells), monocytes, dendritic cells, neutrophils, macrophages, natural killer cells, mast cells, adipocytes, immune cells, neurons, hepatocytes, and cells involved with particular organs (e.g., thymus, endocrine glands, pancreas, brain, neurons, glia, astrocytes, dendrocytes, and genetically modified cells thereof). In some embodiments, a target cell is a cancer cell. In some embodiments, a cell is a liver cell. In some embodiments, a cell is a blood cell. In some embodiments, a cell is a central nervous system cell.

In some embodiments, VLPs are delivered into a cell line such as, but not limited to, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepalclc7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3, . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1, or YAR cells.

### Treatment

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms, and/or in light of genetic or other susceptibility factors). Treatment may also be continued after symptoms have resolved, for example, to prevent or delay their recurrence.

### Variant

As used herein, the term “variant” should be taken to mean the exhibition of qualities that have a pattern that deviates from what occurs in nature, e.g., a variant nucleocapsid protein is a nucleocapsid protein comprising one or more changes in amino acid residues as compared to a wild type nucleocapsid protein amino acid sequence. The term “variant” encompasses homologous proteins having at least 75%, at least 80%, at least 85%, at least 90%, at least 95%, or at least 99% identity with a reference sequence and having the same or substantially the same functional activity or activities as the reference sequence. The term also encompasses mutants, truncations, or domains of a reference sequence that display the same or substantially the same functional activity or activities as the reference sequence.

### Vector

The term “vector,” as used herein, refers to a nucleic acid that can be modified to encode a gene of interest and that is able to enter a host cell, mutate, and replicate within the host cell, and then transfer a replicated form of the vector into another host cell. Exemplary suitable vectors include viral vectors, such as retroviral vectors or bacteriophages and filamentous phage, and conjugative plasmids. Additional suitable vectors will be apparent to those of skill in the art based on the instant disclosure.

### Viral Envelope Glycoprotein

The term “viral envelope glycoprotein” refers to oligosaccharide-containing proteins that form a part of the viral envelope, i.e., the outermost layer of many types of viruses, often comprising lipids, that protects the viral genetic materials when traveling between host cells. Glycoproteins may assist with identification and binding to receptors on a target cell membrane so that the viral envelope fuses with the membrane, allowing the contents of the viral particle (which may comprise, e.g., a cargo as described herein) to enter the host cell. This property may also be referred to as “tropism.” The viral envelope glycoproteins used in the VLPs of the present disclosure may comprise any glycoprotein from an enveloped virus. In some embodiments, a viral envelope glycoprotein is an adenoviral envelope glycoprotein, an adeno-associated viral envelope glycoprotein, a retroviral envelope glycoprotein, or a lentiviral envelope glycoprotein. In certain embodiments, a viral envelope glycoprotein is a vesicular stomatitis virus G protein (VSV-G), a baboon retroviral envelope glycoprotein (BaEVRless), a FuG-B2 envelope glycoprotein, an HIV-1 envelope glycoprotein, or an ecotropic murine leukemia virus (MLV) envelope glycoprotein.

### Virus-Like Particles (VLPs)

As used herein, a “virus-like particle (VLP)” (or “engineered virus-like particle (eVLP),” which is used interchangeably with the term “VLP” herein) consists of a supra-molecular assembly comprising: (a) an envelope comprising (i) a lipid membrane (e.g., single-layer or bi-layer membrane), and a (ii) viral envelope glycoprotein; and (b) a multi-protein core region comprising (ii) a Gag protein, (ii) a first fusion protein comprising a Gag protein and Pro-Pol, and (iii) a second fusion protein comprising a Gag protein fused to a cargo via a protease-cleavable linker. In some embodiments, the gag protein comprises a nucleocapsid protein variant as described herein. In various embodiments, the cargo is a napDNAbp (e.g., Cas9). In other embodiments, the cargo is a base editor. In other embodiments, the cargo is a prime editor. In various other embodiments, the multi-protein core region of the VLPs further comprises one or more guide RNA molecules which are complexed with a napDNAbp, base editor, or prime editor to form a ribonucleoprotein (RNP). In some embodiments, the guide RNA molecule comprises a barcode sequence as described herein (e.g., a barcode sequence associated with a nucleocapsid protein variant that forms part of the gag protein). In various embodiments, the VLPs are prepared in a producer cell that is transiently transformed with plasmid DNA that encodes that various protein and nucleic acid (sgRNA) components of the VLPs. The components self-assemble at the cell membrane and bud out in accordance with the naturally occurring mechanism of retroviral budding in order to release from the cell fully-matured VLPs. Once formed, the Pol-Pro cleaves the protease-sensitive linker joining the Gag-cargo linker (e.g., the linker joining a Gag to a napDNAbp RNP) to release the cargo within the VLP. Once the VLP is administered to a recipient target cell and taken up by said target cell, the contents of the VLP are released, including cargo (e.g., a napDNAbp, base editor, or prime editor). Once in the cell, the cargo may translocate to the nuclease of the cell (in particular, where NLSs are associated with the cargo), where DNA editing may occur at target sites specified by the guide RNA.

In some embodiments, the protease-cleavable linker is optimized to improve cleavage efficiency after VLP maturation. In some embodiments, the Gag-cargo fusion (e.g., Gag::BE) further comprises one or more nuclear export signals at one or more locations along the length of the fusion polypeptide protein which may be joined by a cleavable linker such that during VLP assembly in the producer cell, the Gag-cargo fusions (due to presence of competing NLS signals) do not accumulate in the nucleus of the producer cells but instead are available in the cytoplasm to undergo the VLP assembly process at the cell membrane. Once inside the matured VLPs following release from the producer cell, the NES may be cleaved by Pro-Pol, thereby separating the cargo (e.g., napDNAbp, base editor, prime editor, or other gene editing agent fusion protein) from the NES. Upon delivery to a target cell, therefore, the cargo (e.g., napDNAbp, base editor, or prime editor, typically flanked with one or more NLS elements) will not comprise an NES element, which may otherwise prohibit the transport of the cargo into the nucleus and hinder gene editing activity.

In some embodiments, an optimized stoichiometry ratio of Gag-cargo fusion to Gag-Pro-Pol fusion protein is used, which balances the amount of Gag-cargo available to be packaged into VLPs with the amount of retrovirus protease (the “Pro” in the Gag-Pro-Pol fusion) required for VLP maturation. In some embodiments, the optimized ratio of Gag-cargo fusion to Gag-Pro-Pol fusion protein is achieved by the appropriate ratio of plasmids encoding each component which are transiently delivered to the producer cells. In some embodiments, the gag-cargo:gag-pro-pol stoichiometry is 25% gag-cargo:75% gag-pro-pol.

In some embodiments, a VLP comprises additional agents for targeting the VLP for delivery to particular cell types. For example, such additional targeting agents may be incorporated into the outer lipid membrane encapsulation layer of the VLP. In some embodiments, the additional targeting agent is a protein. In certain embodiments, the additional targeting agent is an antibody or fragment thereof. In certain embodiments, the additional targeting is a ligand (e.g., a receptor ligand). In certain embodiments, the additional targeting agent is a receptor or a fragment thereof. In certain embodiments, the addition targeting agent is an aptamer or a fragment thereof.

Thus, as used herein, a virus-derived particle comprises a virus-like particle formed by one or more virus-derived protein(s), which virus-derived particle is substantially devoid of a viral genome such that the VLP is replication-incompetent when delivered to a target cell.

### Wild Type

As used herein the term “wild type” is a term of the art understood by skilled persons and means the typical form of an organism, strain, gene or characteristic as it occurs in nature as distinguished from mutant or variant forms.

### DETAILED DESCRIPTION OF CERTAIN EMBODIMENTS

The present disclosure describes the development of a platform/system for the directed evolution of virus-like particles (VLPs) with specific desired properties (e.g., increased production levels, increased cargo packaging efficiency, increased transduction of particular target cells of interest, increased infectivity, increased stability, and/or decreased immunogenicity). This platform was used to evolve several next-generation VLP variants with such desired properties, in particular, with increased production levels, increased cargo packaging efficiency, and/or increased transduction of particular target cells of interest. VLPs have been engineered previously, for example, to deliver Cas9, base editors, and prime editors. See, for example, International Patent Application Nos. PCT/US2022/080834, PCT/US2022/080836, and PCT/US2022/080856, each of which was filed on Dec. 2, 2022. These VLPs, however, utilize wild type viral gag proteins (including wild type nucleocapsid proteins) and wild type envelope glycoproteins, which have been optimized by natural evolution to package viral genomes rather than desired cargo molecules (e.g., gene editing agents such as napDNAbp, base editors, and prime editors). Because VLPs lack genetic material, traditional directed evolution schemes (such as those commonly used for evolving viruses with desired properties) cannot be used. The platform described herein allows the wild type VLP components, such as the viral nucleocapsid protein, to be evolved to more efficiently package alternative cargos.

Accordingly, the present disclosure provides methods for evolving VLPs having one or more desired properties. The present disclosure also provides libraries of polynucleotides and libraries of cells for use in such methods, and methods for producing the libraries. Group specific antigen (gag) proteins comprising nucleocapsid protein variants evolved using the methods described herein are also provided herein. The present disclosure also provides VLPs comprising such gag proteins comprising nucleocapsid protein variants. Polynucleotides, vectors, cells, and kits useful for performing the methods and/or encoding the gag protein variants described herein are also provided.

### Libraries and Methods for Evolving VLPs

In some aspects, the present disclosure provides methods for evolving VLPs, libraries for use in such methods, and methods of producing such libraries. As described herein, the inventors discovered that VLP libraries comprising variants of at least one component of the VLP, such as the nucleocapsid protein portion of the gag protein, could be produced in such a way that each VLP variant packages a guide RNA (gRNA) containing a barcode sequence that encodes the identity of that particular VLP variant. In this barcoded VLP library approach, each variant (e.g., nucleocapsid protein variant) is paired with a unique barcode sequence on a vector that is packaged into the VLP. These barcoded vectors are then used to produce barcoded VLPs in a pooled fashion, ensuring that each VLP variant only packages its cognate barcode, maintaining the specified variant:barcode association. After applying a selection for the desired properties, the barcoded molecules (e.g., barcoded gRNAs) are sequenced, and the enriched barcodes are quantified, enabling identification of VLPs that possess the desired properties.

Thus, in one aspect, the present disclosure provides methods for generating a library of cells capable of producing VLPs comprising transfecting a plurality of polynucleotides encoding VLPs (e.g., the gag-cargo fusion, the gag-pro polyprotein, the envelope glycoprotein, and a barcoded nucleic acid molecule as described herein) into producer cells, wherein each polynucleotide encodes a variant of at least one component of the VLP or its cargo, wherein the variant comprises at least one amino acid substitution relative to a VLP that does not comprise a variant of at least one component of the VLP or its cargo; and wherein each polynucleotide encoding a variant of a VLP component or cargo is associated with a unique nucleic acid barcode sequence.

In another aspect, the present disclosure provides methods for generating a library of VLPs comprising (i) transfecting a plurality of polynucleotides encoding VLPs (e.g., the gag-cargo fusion, the gag-pro polyprotein, the envelope glycoprotein, and a barcoded nucleic acid molecule as described herein) into producer cells, wherein each polynucleotide encodes a variant of at least one component of the VLP or its cargo, wherein the variant comprises at least one amino acid substitution relative to a VLP that does not comprise a variant of at least one component of the VLP or its cargo; and wherein each polynucleotide encoding a VLP component or cargo variant is associated with a unique nucleic acid barcode sequence encoded by the polynucleotide (e.g., on a nucleic acid molecule, such as a gRNA, that is packaged into the VLP comprising that particular variant); (ii) producing VLPs from the producer cells; and (iii) isolating the library of VLPs.

In another aspect, the present disclosure provides methods for evolving VLPs comprising (i) transfecting a plurality of polynucleotides encoding VLPs (e.g., the gag-cargo fusion, the gag-pro polyprotein, the envelope glycoprotein, and a barcoded nucleic acid molecule as described herein) into producer cells, wherein each polynucleotide encodes a variant of at least one component of the VLP or its cargo, wherein the variant comprises at least one amino acid substitution relative to a VLP that does not comprise a variant of at least one component of the VLP or its cargo; and wherein each polynucleotide encoding a VLP component or cargo variant is associated with a unique nucleic acid barcode sequence encoded by the polynucleotide (e.g., on a nucleic acid molecule, such as a gRNA, that is packaged into the VLP comprising that particular variant); (ii) producing VLPs from the producer cells; and (iii) selecting VLPs having at least one improved property by determining the abundance of each barcode sequence relative to the abundance of a barcode sequence associated with a VLP that does not comprise a variant of at least one component of the VLP or its cargo; wherein VLPs comprising variants associated with barcode sequences that are present at higher abundance relative to the barcode sequence associated with the VLP that does not comprise a variant of at least one component of the VLP or its cargo have at least one improved property.

The present disclosure contemplates the evolution of VLPs comprising a variant of any component of the VLP, including the viral envelope glycoprotein, the gag-pro-polyprotein, or the gag protein (which includes the viral nucleocapsid protein). In some embodiments, the present disclosure contemplates the evolution of VLPs comprising a variant nucleocapsid protein portion of the gag protein. The present disclosure also contemplates the evolution of VLPs comprising a variant of a cargo that is packaged into the VLP (e.g., a protein or fusion protein such as a napDNAbp, a base editor, or a prime editor). In some embodiments, the VLPs evolved herein comprise variants of more than one component and/or cargo of the VLP.

In certain embodiments, the VLPs evolved using the methods described herein comprise a variant of the viral nucleocapsid protein. Such variants can be obtained, for example, from commercial vendors that produce and sell short DNA sequences. Sequences encoding all possible single-codon mutants of a viral nucleocapsid protein as an oligonucleotide pool can thus be obtained. Any method of mutagenesis known in the art (e.g., error-prone PCR, site-saturation mutagenesis, etc.) can also be used to generate VLP nucleocapsid protein variants, or variants of any other VLP component or cargo.

VLPs having various improved properties can be obtained using the methods provided herein. For example, the methods can be used to obtain VLPs that have a higher efficiency of packaging one or more cargo molecules and/or increased production in the producer cells. Such VLPs may be obtained by sequencing the barcode sequences after VLPs have been produced by producer cells and determining which barcode sequences are observed at the highest abundance. The barcode sequences with the highest abundance represent VLPs comprising variant components (e.g., a variant nucleocapsid protein) that were produced more efficiently and thus are present at higher levels.

The methods provided herein can also be used to evolve VLPs that have improved transduction efficiency into a particular target cell type. In some embodiments, a method further comprises isolating the VLPs from the producer cells, and transducing the VLPs into a target cell type prior to performing the selection. After the VLPs have been transduced into a target cell type of interest, the barcodes can be sequenced, and the barcodes that are present at higher abundance are determined to correspond to VLPs comprising variant components (e.g., a variant nucleocapsid protein) that were transduced into the target cell more efficiently and thus are present at higher levels. In some embodiments, VLPs are delivered into stem cells (e.g., human stem cells) such as, for example, pluripotent stem cells (e.g., human pluripotent stem cells including human induced pluripotent stem cells (hiPSCs)). In some embodiments, VLPs are delivered into any of the other cell types provided herein.

In some embodiments, the methods provided herein are used for evolving VLPs that are optimized for packaging a particular type of cargo molecule with high efficiency. In certain embodiments, the VLPs are optimized to package base editors, or a particular base editor of interest. In certain embodiments, the VLPs are optimized to package prime editors, or a particular prime editor of interest. In certain embodiments, the VLPs may be optimized to package any gene editing agent of interest.

In some embodiments, the methods provided herein are used for evolving the envelope proteins of a VLP in order to bias VLP transduction toward particular target cell types of interest over other non-target cell types. In some embodiments, the methods provided herein are used for evolving any surface-exposed targeting moiety of a VLP in order to bias VLP transduction toward particular target cell types of interest over other non-target cell types.

In some embodiments, polynucleotides encoding the VLPs are first packaged into viral particles prior to being transfected into producer cells. In some embodiments, VLPs are first packaged into lentiviral particles to produce a lentiviral library, and the lentiviral library is then transduced into producer cells. In some embodiments, polynucleotides (e.g., plasmids or mRNA) encoding the VLPs are transfected directly into producer cells.

Once polynucleotides encoding the VLPs have been transfected or transduced into producer cells, it is important that each producer cell is only capable of producing a single VLP library member (e.g., a VLP comprising a particular nucleocapsid protein variant). This ensures that the nucleocapsid protein variant and the barcode will remain associated with one another and be specific throughout the selection process. Thus, in some embodiments, a low multiplicity of infection is used when transfecting or transducing the polynucleotides encoding VLPs (e.g., the gag-cargo fusion, the gag-pro polyprotein, the envelope glycoprotein, and a barcoded nucleic acid molecule as described herein) into producer cells, ensuring that producer cells will be capable of encoding either zero or one VLP variant. In some embodiments, the methods provided herein further comprise a step of selecting for producer cells that contain polynucleotides encoding a VLP. In certain embodiments, such a selection comprises an antibiotic selection.

In some embodiments, the barcode sequence is included on a nucleic acid cargo molecule that is packaged into the VLP. In some embodiments, the nucleic acid cargo molecule is a gRNA. It has been demonstrated that <25 base pair barcode sequences can be inserted into the guide RNA at the P1 stem/tetraloop, without disrupting Cas9 binding or function (Zhu, Wei et al., Genome Biol. 20, 20 (2019); Shechner, Rinn et al., Nat. Methods 12, 664-670 (2015)). The barcode association with a particular variant (e.g., a nucleocapsid protein variant) must be maintained during VLP production. This requires only one library member to be introduced per producer cell, as discussed above. With only one library member per producer cell, each cell produces a single VLP variant packaging a single cognate barcode sequence, thereby maintaining a specific barcode association with each variant.

In some embodiments, a barcode sequence on a gRNA is associated with a particular variant of a viral nucleocapsid protein in a VLP. Such barcodes can be packaged into a VLP as it is produced and then sequenced and used, for example, to determine which VLPs comprise variants of a nucleocapsid protein or other component that confer one or more improved properties relative to a VLP that comprises a wild type nucleocapsid protein or other component of the VLP. In some embodiments, a barcode sequence is inserted in a portion of a gRNA outside of the Cas9-gRNA ribonucleoprotein complex (i.e., in a portion of the gRNA to which Cas9 does not bind). In some embodiments, a barcode sequence is inserted into the P1 stem/tetraloop portion of a gRNA. In some embodiments, a barcode sequence is 5-25, 6-24, 7-23, 8-22, 9-21, 10-20, 11-19, 12-18, 13-17, or 14-16 nucleotides long. In some embodiments, a barcode sequence is 5, 6, 7, 8, 9, 10, 11, 12, 13, 14, 15, 16, 17, 18, 19, 20, 21, 22, 23, 24, or 25 nucleotides long. In certain embodiments, a barcode sequence is 15 nucleotides long. Such gRNAs comprising a barcode sequence may be included on a plasmid or other vector along with a variant of a VLP component (such as a nucleocapsid protein variant) in the libraries provided herein. Each barcode sequence is thus associated with a particular variant, and as long as the association is maintained throughout the selection process, the barcode sequence will remain associated with a particular variant and allow identification of that variant (e.g., by sequencing) following selection.

In some embodiments, each library member transfected into producer cells comprises a polynucleotide that encodes 1) a viral nucleocapsid protein variant, and 2) a guide RNA comprising a unique nucleic acid barcode sequence encoding the identity of the viral nucleocapsid protein variant. In some embodiments, the polynucleotide further comprises a selection marker. In certain embodiments, the selection marker is an antibiotic resistance gene. In some embodiments, each library member transfected into a producer cell further comprises one or more additional polynucleotides encoding other components of the VLP, as described further below. In certain embodiments, polynucleotides encoding the various components of the VLPs are provided on four different vectors. In certain embodiments, polynucleotides encoding the various components of the VLPs are provided on three different vectors.

In another aspect, the present disclosure provides libraries of polynucleotides encoding VLPs (e.g., the gag-cargo fusion, the gag-pro polyprotein, the envelope glycoprotein, and a barcoded nucleic acid molecule as described herein), wherein each library member comprises a polynucleotide encoding a variant of at least one component of the VLP or its cargo comprising at least one amino acid substitution relative to a VLP that does not comprise a variant of at least one component of the VLP or its cargo, and wherein each polynucleotide encoding a VLP component or cargo variant is associated with a unique nucleic acid barcode sequence encoded by the polynucleotide (e.g., on a nucleic acid molecule, such as a gRNA, that is packaged into the VLP comprising that particular variant). Such libraries may be useful for performing the methods for evolving VLPs described herein.

In another aspect, the present disclosure provides libraries of cells capable of producing virus-like particles (VLPs), wherein each producer cell comprises a polynucleotide encoding a variant of at least one component of a VLP or its cargo comprising at least one amino acid substitution relative to a VLP that does not comprise a variant of at least one component of the VLP or its cargo, and wherein each polynucleotide encoding a VLP component or cargo variant is associated with a unique nucleic acid barcode sequence encoded by the polynucleotide (e.g., on a nucleic acid molecule, such as a gRNA, that is packaged into the VLP comprising that particular variant). Such libraries may be useful for performing the methods for evolving VLPs described herein.

### VLP Components

In various embodiments, the VLPs that make up the libraries used in the methods provided herein comprise a supra-molecular assembly comprising (a) an envelope comprising (i) a lipid membrane (e.g., single-layer or bi-layer membrane), and a (ii) viral envelope glycoprotein; and (b) a multi-protein core region enclosed by the envelope and comprising (i) a Gag protein, (ii) a Gag-Pro-Pol protein, and (iii) a Gag-cargo fusion protein comprising a Gag protein fused to a cargo (e.g., a cargo protein such as a napDNAbp, base editor, or prime editor) via a cleavable linker (e.g., a protease-cleavable linker). The gag protein comprises, among other components, a viral nucleocapsid protein (e.g., a variant viral nucleocapsid protein as described herein). In various embodiments, the cargo is a napDNAbp (e.g., Cas9). In other embodiments, the cargo is a base editor. In other embodiments, the cargo is a prime editor. In various other embodiments, the multi-protein core region of the VLPs further comprises one or more nucleic acid molecules comprising a unique barcode sequence as described herein. In various other embodiments, the multi-protein core region of the VLPs further comprises one or more guide RNA molecules (e.g., comprising a unique barcode sequence as described herein) which are complexed with the napDNAbp, base editor, or prime editor to form a ribonucleoprotein (RNP). In various embodiments, the VLPs are prepared in a producer cell that is transiently transformed with plasmid DNA that encodes the various protein and nucleic acid (gRNA) components of the VLPs. Without being bound by theory, the components self-assemble at the cell membrane and bud out in accordance with the naturally occurring mechanism of budding (e.g., retroviral budding or the budding mechanism of other envelope viruses) in order to release from the cell fully-matured VLPs. Once formed, the Gag-Pro-Pol cleaves the protease-sensitive linker of the Gag-cargo (i.e., [Gag]-[cleavable linker]-[cargo], wherein the cargo may in some embodiments be base editor RNP, prime editor RNP, or a napDNAbp RNP), thereby releasing the RNP or other cargo molecule within the VLP. Thus, in various embodiments, the present disclosure also provides VLPs in which the cargo has been cleaved off of the gag protein and released within the VLP. For example, the present disclosure provides VLPs comprising a group-specific antigen (gag) protease (pro) polyprotein, a cargo such as a cargo protein, and a fusion protein comprising a gag nucleocapsid protein and a nuclear export sequence (NES), encapsulated by a lipid membrane and a viral envelope glycoprotein. In some embodiments, the present disclosure provides VLPs comprising a mixture of cleaved and uncleaved products (i.e., a mixture of cargos that have been cleaved from the gag protein, and that have not yet been cleaved from the gag protein). In some embodiments, the cargo is fused to one or more additional domains such as one or more NLS.

Once the VLP is administered to a target cell and taken up by said target cell, the contents of the VLP are released. Once in the cell, the cargo may translocate to the nucleus of the cell (in particular, where NLSs are included on the cargo), where DNA editing may occur at target sites specified by a guide RNA. In some embodiments, the Gag-cargo fusion further comprises one or more nuclear export signals at one or more locations along the length of the fusion polypeptide protein which may be joined by a cleavable linker such that during VLP assembly in the producer cell, the Gag-cargo fusions (due to presence of competing NLS signals) do not accumulate in the nucleus of the producer cells but instead are available in the cytoplasm to undergo the VLP assembly process at the cell membrane. Once inside the matured VLPs following release from the producer cell, the NES may be cleaved by Gag-Pro-Pol thereby separating the cargo from the NES. Upon delivery to a target cell, therefore, the cargo will not comprise an NES element, which may otherwise prohibit the transport of the cargo into the nucleus and hinder gene editing activity.

In some embodiments, the VLPs comprise a gag-cargo:gag-pro-pol stoichiometry of 25% gag-cargo: 75% gag-pro-pol. In some embodiments, the ratio of gag-pro-polyprotein to gag-cargo is approximately 10:1, approximately 9:1, approximately 8:1, approximately 7:1, approximately 6:1, approximately 5:1, approximately 4:1, approximately 3:1, approximately 2:1, approximately 1.5:1, approximately 1:1, or approximately 0.5:1.

Accordingly, in some embodiments, the VLPs of the present disclosure comprise (a) an envelope, and (b) a multi-protein core, wherein the envelope comprises a lipid membrane (e.g., a lipid mono- or bi-layer membrane) and a viral envelope glycoprotein, and wherein the multi-protein core comprises a Gag (e.g., a retroviral Gag), a group-specific antigen (gag) protease (pro) polyprotein (i.e., “Gag-Pro-Pol”), and a fusion protein comprising a Gag-cargo (e.g., Gag-napDNAbp, Gag-base editor, or Gag-prime editor). In various embodiments, the Gag-cargo may comprise a ribonucleoprotein cargo, e.g., a napDNAbp, base editor, or prime editor complexed with a guide RNA. In still further embodiments, the Gag-cargo may comprise one or more NLS sequences and/or one or more NES sequences to regulate the cellular location of the cargo in a cell. An NLS sequence will facilitate the transport of the cargo into the cell's nucleus to facilitate editing. An NES will do the opposite, i.e., transport the cargo out from the nucleus, and/or prevent the transport of the cargo into the nucleus. In certain embodiments, the NES, or multiple NES, may be coupled to the fusion protein by a cleavable linker (e.g., a protease linker) such that during assembly in a producer cell, the one or more NES operate to keep the cargo in the cytoplasm and available for the packaging process. However, once matured VLPs are budded out or released from a producer cell in a mature form, the cleavable linker joining the NES may be cleaved, thereby removing the association of NES with the cargo. Thus, without an NES, the cargo will translocate to the nucleus with its NLS sequences, thereby facilitating editing. Various napDNAbps may be used in the systems of the present disclosure. In some embodiments, the napDNAbp is a Cas9 protein (e.g., a Cas9 nickase, dead Cas9 (dCas9), or another Cas9 variant). In some embodiments, the Cas9 protein is bound to a guide RNA (gRNA). The fusion protein may further comprise other protein domains, such as effector domains. In some embodiments, the fusion protein comprises a deaminase domain (e.g., an adenosine deaminase domain or a cytosine deaminase domain). In certain embodiments, the fusion protein comprises a base editor, such as ABE8e, or any of the other base editors described herein or known in the art. In certain embodiments, the fusion protein comprises a polymerase (e.g., a reverse transcriptase). In certain embodiments, the fusion protein comprises a prime editor as described herein, or any prime editor known in the art.

In some embodiments, the fusion protein comprises more than one NES (e.g., two NES, three NES, four NES, five NES, six NES, seven NES, eight NES, nine NES, or ten or more NES). In certain embodiments, the fusion protein further comprises a nuclear localization sequence (NLS), or more than one NLS (e.g., two NLS, three NLS, four NLS, five NLS, six NLS, seven NLS, eight NLS, nine NLS, or ten or more NLS). In certain embodiments, the fusion protein may comprise at least one NES and one NLS. In certain embodiments, the fusion protein comprises at least one more NES than NLS (e.g., three NES and one or two NLS).

The Gag-cargo fusion proteins described herein comprise one or more cleavable linkers. In one embodiment, the Gag-cargo fusion proteins comprise a cleavable linker joining the Gag to the cargo, such that once the Gag-cargo fusion has been packaged in mature VLPs (which will also contain the Gag-Pro-Pol), the protease activity can cleave the Gag-cargo cleavable linker, thereby releasing the cargo. In some embodiments, a cleavable linker may also be provided in such a location such that when the cleavable linker is cleaved (e.g., by the Gag-Pro-Pol protein), the NES is separated away from the cargo. Such an arrangement of the fusion protein allows the fusion protein to be exported from the nucleus of a producing cell during VLP production, and the NES can later be cleaved from the fusion protein after delivery to a target cell, or prior to delivery to the target cell but after packaging into the VLP, releasing the cargo and allowing it to enter the nucleus of the target cell. In some embodiments, the cleavable linker comprises a protease cleavage site (e.g., a Moloney murine leukemia virus (MMLV) protease cleavage site or a Friend murine leukemia virus (FMLV) protease cleavage site). Various protease cleavage sites can be used in the fusion proteins of the present disclosure. In certain embodiments, the protease cleavage site comprises the amino acid sequence TSTLLMENSS (SEQ ID NO: 1), PRSSLYPALTP (SEQ ID NO: 2), VQALVLTQ (SEQ ID NO: 3), PLQVLTLNIERR (SEQ ID NO: 4), or an amino acid sequence at least 90% identical to any one of SEQ ID NOs: 1-4. In some embodiments, the protease cleavage site comprises the amino acid sequence of any one of SEQ ID NOs: 1-4 comprising one mutation, two mutations, three mutations, four mutations, five mutations, or more than five mutations relative to one of SEQ ID NOs: 1-4. In some embodiments, the cleavable linker of the fusion protein is cleaved by the protease of the gag-pro polyprotein. In certain embodiments, the cleavable linker of the fusion protein is not cleaved by the protease of the gag-pro polyprotein until the VLP has been assembled and delivered into a target cell. In some embodiments, the gag-pro polyprotein of the VLPs described herein comprises an MMLV gag-pro polyprotein or an FMLV gag-pro polyprotein. In some embodiments, the gag nucleocapsid protein of the fusion protein in the VLPs described herein comprises an MMLV gag nucleocapsid protein or an FMLV gag nucleocapsid protein.

In certain embodiments, the fusion protein comprises the following non-limiting structures:
[gag nucleocapsid protein variant]-[1×-3×NES]-[cleavable linker]-[cargo], wherein each instance of]-[independently comprises an optional linker (e.g., an amino acid linker, or any of the linkers provided herein); or
[1×-3×NES]-[gag nucleocapsid protein variant]-[cleavable linker]-[cargo], wherein each instance of]-[independently comprises an optional linker (e.g., an amino acid linker, or any of the linkers provided herein).

In embodiments in which the cleavable linker has been cleaved by the protease within the VLP, the VLP may comprise a fusion protein comprising the structure: [gag nucleocapsid protein]-[1×-3×NES], and a free cargo.

In some embodiments, any of the constructs described herein comprise 1×NES. In some embodiments, any of the constructs described herein comprise 2×NES. In some embodiments, any of the constructs described herein comprise 3×NES. In some embodiments, any of the constructs described herein comprise 4×NES. In some embodiments, any of the constructs described herein comprise 5×NES.

The VLPs provided by the present disclosure comprise an outer encapsulation layer (or envelope layer) comprising a viral envelope glycoprotein. Any viral envelope glycoprotein described herein, or known in the art, may be used in the VLPs of the present disclosure. In some embodiments, the viral envelope glycoprotein is an adenoviral envelope glycoprotein, an adeno-associated viral envelope glycoprotein, a retroviral envelope glycoprotein, or a lentiviral envelope glycoprotein. In certain embodiments, the viral envelope glycoprotein is a retroviral envelope glycoprotein. In some embodiments, the viral envelope glycoprotein is a vesicular stomatitis virus G protein (VSV-G), a baboon retroviral envelope glycoprotein (BaEVRless), a FuG-B2 envelope glycoprotein, an HIV-1 envelope glycoprotein, or an ecotropic murine leukemia virus (MLV) envelope glycoprotein. In some embodiments, the viral envelope glycoprotein targets the system to a particular cell type (e.g., immune cells, neural cells, retinal pigment epithelium cells, etc.). For example, using different envelope glycoproteins in the VLPs described herein may alter their cellular tropism, allowing the VLPs to be targeted to specific cell types. In some embodiments, the viral envelope glycoprotein is a VSV-G protein, and the VSV-G protein targets the system to retinal pigment epithelium (RPE) cells. In some embodiments, the viral envelope glycoprotein is an HIV-1 envelope glycoprotein, and the HIV-1 envelope glycoprotein targets the system to CD4+ cells. In some embodiments, the viral envelope glycoprotein is a FuG-B2 envelope glycoprotein, and the FuG-B2 envelope glycoprotein targets the system to neurons.

It will be appreciated that general methods are known in the art for producing viral vector particles, which generally contain coding nucleic acids of interest, and may also be used for producing the virus-derived particles according to the present invention, which do not contain coding nucleic acids of interest but instead are designed to deliver a protein cargo.

Conventional viral vector particles encompass retroviral, lentiviral, adenoviral, and adeno-associated viral vector particles that are well known in the art. For a review of various viral vector particles that may be used, the one skilled in the art may notably refer to Kushnir et al. (2012, Vaccine, Vol. 31: 58-83), Zeltons (2013, Mol Biotechnol, Vol. 53: 92-107), Ludwig et al. (2007, Curr Opin Biotechnol, Vol. 18(no 6): 537-55) and Naskalaska et al. (2015, Vol. 64 (no 1): 3-13). Further, references to various methods using virus-derived particles for delivering proteins to cells are found by the one skilled in the art in the article of Maetzig et al. (2012, Current Gene Therapy, Vol. 12: 389-409), as well as the article of Kaczmarczyk et al. (2011, Proc Natl Acad Sci USA, Vol. 108 (no 41): 16998-17003).

Generally, a virus-like particle that is used according to the present disclosure, which virus-like particle may also be termed “virus-derived particle,” is formed by one or more virus-derived structural protein(s) and/or one more virus-derived envelope protein.

A virus-like particle that is used according to the present invention is replication incompetent in a host cell wherein it has entered.

In preferred embodiments, a virus-like particle is formed by one or more retrovirus-derived structural protein(s) and optionally one or more virus-derived envelope protein(s).

In preferred embodiments, the virus-derived structural protein is a retroviral Gag protein or a peptide fragment thereof. As it is known in the art, Gag and Gag/pol precursors are expressed from full length genomic RNA as polyproteins, which require proteolytic cleavage, mediated by the retroviral protease (PR), to acquire a functional conformation. Further, Gag, which is structurally conserved among the retroviruses, is composed of at least three protein units: matrix protein (MA), capsid protein (CA) and nucleocapsid protein (NC), whereas Pol consists of the retroviral protease, (PR), the retrotranscriptase (RT), and the integrase (IN).

In some embodiments, a virus-derived particle comprises a retroviral Gag protein but does not comprise a Pol protein.

As it is known in the art, the host range of retroviral vector, including lentiviral vectors, may be expanded or altered by a process known as pseudotyping. Pseudotyped lentiviral vectors consist of viral vector particles bearing glycoproteins derived from other enveloped viruses. Such pseudotyped viral vector particles possess the tropism of the virus from which the glycoprotein is derived.

In some embodiments, a virus-like particle is a pseudotyped virus-like particle comprising one or more viral structural protein(s) or viral envelope protein(s) imparting a tropism to the said virus-like particle for certain eukaryotic cells. A pseudotyped virus-like particle as described herein may comprise, as the viral protein used for pseudotyping, a viral envelope protein selected in a group comprising VSV-G protein, Measles virus HA protein, Measles virus F protein, Influenza virus HA protein, Moloney virus MLV-A protein, Moloney virus MLV-E protein, Baboon Endogenous retrovirus (BAEV) envelope protein, Ebola virus glycoprotein, and foamy virus envelope protein, or a combination of two or more of these viral envelope proteins.

A well-known illustration of pseudotyping viral vector particles consists of the pseudotyping of viral vector particles with the vesicular stomatitis virus glycoprotein (VSV-G). For the pseudotyping of viral vector particles, one skilled in the art may notably refer to Yee et al. (1994, Proc Natl Acad Sci, USA, Vol. 91: 9564-9568) Cronin et al. (2005, Curr Gene Ther, Vol. 5(no 4): 387-398), which are incorporated herein by reference.

For producing virus-like particles, and more precisely VSV-G pseudotyped virus-like particles, for delivering protein(s) of interest into target cells, one skilled in the art may refer to Mangeot et al. (2011, Molecular Therapy, Vol. 19 (no 9): 1656-1666).

In some embodiments, a virus-like particle further comprises a viral envelope protein, wherein either (i) the said viral envelope protein originates from the same virus as the viral structural protein, e.g., originates from the same virus as the viral Gag protein, or (ii) the said viral envelope protein originates from a virus distinct from the virus from which originates the viral structural protein, e.g., originates from a virus distinct from the virus from which originates the viral Gag protein.

As is readily understood by one skilled in the art, a virus-like particle that is used according to the disclosure may be selected in a group comprising Moloney murine leukemia virus-derived vector particles, Bovine immunodeficiency virus-derived particles, Simian immunodeficiency virus-derived vector particles, Feline immunodeficiency virus-derived vector particles, Human immunodeficiency virus-derived vector particles, Equine infection anemia virus-derived vector particles, Caprine arthritis encephalitis virus-derived vector particle, Baboon endogenous virus-derived vector particles, Rabies virus-derived vector particles, Influenza virus-derived vector particles, Norovirus-derived vector particles, Respiratory syncytial virus-derived vector particles, Hepatitis A virus-derived vector particles, Hepatitis B virus-derived vector particles, Hepatitis E virus-derived vector particles, Newcastle disease virus-derived vector particles, Norwalk virus-derived vector particles, Parvovirus-derived vector particles, Papillomavirus-derived vector particles, Yeast retrotransposon-derived vector particles, Measles virus-derived vector particles, and bacteriophage-derived vector particles.

In particular, a virus-like particle that is used according to the invention is a retrovirus-derived particle. Such retrovirus may be selected among Moloney murine leukemia virus, Bovine immunodeficiency virus, Simian immunodeficiency virus, Feline immunodeficiency virus, Human immunodeficiency virus, Equine infection anemia virus, and Caprine arthritis encephalitis virus.

In another embodiment, a virus-like particle that is used according to the disclosure is a lentivirus-derived particle. Lentiviruses belong to the retroviruses family and have the unique ability of being able to infect non-dividing cells.

Such lentivirus may be selected among Bovine immunodeficiency virus, Simian immunodeficiency virus, Feline immunodeficiency virus, Human immunodeficiency virus, Equine infection anemia virus, and Caprine arthritis encephalitis virus.

For preparing Moloney murine leukemia virus-derived vector particles, one skilled in the art may refer to the methods disclosed by Sharma et al. (1997, Proc Natl Acad Sci USA, Vol. 94: 10803+-10808), Guibingua et al. (2002, Molecular Therapy, Vol. 5(no 5): 538-546), which are incorporated herein by reference. Moloney murine leukemia virus-derived (MLV-derived) vector particles may be selected in a group comprising MLV-A-derived vector particles and MLV-E-derived vector particles.

For preparing Bovine Immunodeficiency virus-derived vector particles, one skilled in the art may refer to the methods disclosed by Rasmussen et al. (1990, Virology, Vol. 178(no 2): 435-451), which is incorporated herein by reference.

For preparing Simian immunodeficiency virus-derived vector particles, including VSV-G pseudotyped SIV virus-derived particles, one skilled in the art may notably refer to the methods disclosed by Mangeot et al. (2000, Journal of Virology, Vol. 71(no. 18): 8307-8315), Negre et al. (2000, Gene Therapy, Vol. 7: 1613-1623) Mangeot et al. (2004, Nucleic Acids Research, Vol. 32 (no. 12), e102), which are incorporated herein by reference.

For preparing Feline Immunodeficiency virus-derived vector particles, one skilled in the art may notably refer to the methods disclosed by Saenz et al. (2012, Cold Spring Harb Protoc, (1): 71-76; 2012, Cold Spring Harb Protoc, (1): 124-125; 2012, Cold Spring Harb Protoc, (1): 118-123), which are incorporated herein by reference.

For preparing Human immunodeficiency virus-derived vector particles, one skilled in the art may notably refer to the methods disclosed by Jalaguier et al. (2011, PlosOne, Vol. 6(no. 11), e28314), Cervera et al. (J. Biotechnol., Vol. 166(no. 4): 152-165), Tang et al. (2012, Journal of Virology, Vol. 86(no. 14): 7662-7676), which are incorporated herein by reference.

For preparing Equine infection anemia virus-derived vector particles, one skilled in the art may notably refer to the methods disclosed by Olsen (1998, Gene Ther, Vol. 5(no 11): 1481-1487), which are incorporated herein by reference.

For preparing Caprine arthritis encephalitis virus-derived vector particles, one skilled in the art may notably refer to the methods disclosed by Mselli-Lakhal et al. (2006, J Virol Methods, Vol. 136(no. 1-2): 177-184), which are incorporated herein by reference.

For preparing Baboon endogenous virus-derived vector particles, one skilled in the art may notably refer to the methods disclosed by Girard-Gagnepain et al. (2014, Blood, Vol. 124(no. 8): 1221-1231), which is incorporated herein by reference.

For preparing Rabies virus-derived vector particles, one skilled in the art may notably refer to the methods disclosed by Kang et al. (2015, Viruses, Vol. 7: 1134-1152, doi:10.3390/v7031134), Fontana et al. (2014, Vaccine, Vol. 32(no. 24): 2799-27804) or to the PCT application published under no WO 2012/0618, which is incorporated herein by reference.

For preparing Influenza virus-derived vector particles, one skilled in the art may notably refer to the methods disclosed by Quan et al. (2012, Virology, Vol. 430: 127-135) and to Latham et al. (2001, Journal of Virology, Vol. 75(no. 13): 6154-6155), which is incorporated herein by reference.

For preparing Norovirus-derived vector particles, one skilled in the art may notably refer to the methods disclosed by Tom6-Amat et al., (2014, Microbial Cell Factories, Vol. 13: 134-142), which is incorporated herein by reference.

For preparing Respiratory syncytial virus-derived vector particles, one skilled in the art may notably refer to the methods disclosed by Walpita et al. (2015, PlosOne, DOI: 10.1371/journal.pone.0130755), which is incorporated herein by reference.

For preparing Hepatitis B virus-derived vector particles, one skilled in the art may notably refer to the methods disclosed by Hong et al. (2013, Vol. 87(no. 12): 6615-6624), which is incorporated herein by reference.

For preparing Hepatitis E virus-derived vector particles, one skilled in the art may notably refer to the methods disclosed by Li et al. (1997, Journal of Virology, Vol. 71(no 10): 7207-7213), which is incorporated herein by reference.

For preparing Newcastle disease virus-derived vector particles, one skilled in the art may notably refer to the methods disclosed by Murawski et al. (2010, Journal of Virology, Vol. 84(no. 2): 1110-1123), which is incorporated herein by reference.

For preparing Norwalk virus-derived vector particles, one skilled in the art may notably refer to the methods disclosed by Herbst-Kralovetz et al. (2010, Expert Rev Vaccines, Vol. 9(no 3): 299-307), which is incorporated herein by reference.

For preparing Parvovirus-derived vector particles, one skilled in the art may notably refer to the methods disclosed by Ogasawara et al. (2006, In Vivo, Vol. 20: 319-324), which is incorporated herein by reference.

For preparing Papillomavirus-derived vector particles, one skilled in the art may notably refer to the methods disclosed by Wang et al. (2013, Expert Rev Vaccines, Vol. 12(no. 2): doi:10.1586/erv.12.151), which is incorporated herein by reference.

A virus-like particle that is used herein comprises a Gag protein, and most preferably a Gag protein originating from a virus selected from a group consisting of Rous Sarcoma Virus (RSV), Feline Immunodeficiency Virus (FIV), Simian Immunodeficiency Virus (SIV), Moloney Leukemia Virus (MLV), and Human Immunodeficiency Viruses (HIV-1 and HIV-2), especially Human Immunodeficiency Virus of type 1 (HIV-1).

In some embodiments, a virus-like particle may also comprise one or more viral envelope protein(s). The presence of one or more viral envelope protein(s) may impart to the said virus-derived particle a more specific tropism for the cells which are targeted, as it is known in the art. The one or more viral envelope protein(s) may be selected from a group consisting of envelope proteins from retroviruses, envelope proteins from non-retroviral viruses, and chimeras of these viral envelope proteins with other peptides or proteins. An example of a non-lentiviral envelope glycoprotein of interest is the lymphocytic choriomeningitis virus (LCMV) strain WE54 envelope glycoprotein. These envelope glycoproteins increase the range of cells that can be transduced with retroviral derived vectors.

### Evolved Nucleocapsid Proteins and VLPs

In some aspects, the present disclosure provides nucleocapsid proteins and VLPs evolved using the methods provided herein. As described herein, quantification of barcode enrichment in VLPs comprising nucleocapsid protein variants relative to producer cells in the screening methods provided herein was performed, and enriched barcodes were determined to correspond to variants that possess improved titer or cargo packaging compared to VLPs and/or improved transduction efficiency into one or more target cell types of interest.

In one aspect, the present disclosure provides group specific antigen (gag) proteins comprising viral nucleocapsid protein variants evolved using the methods described herein. In some embodiments, the gag protein (which comprises the viral nucleocapsid protein) is a variant that is at least 70%, at least 75%, at least 80%, at least 85%, at least 90%, at least 95%, at least 96%, at least 97%, at least 98%, or at least 99% identical to the amino acid sequence of SEQ ID NO: 100:

(SEQ ID NO: 100)
MGQTVTTPLSLTLGHWKDVERIAHNQSVDVKKRRWVTFCSAEWPT
FNVGWPRDGTFNRDLITQVKIKVFSPGPHGHPDQVPYIVTWEALA
FDPPPWVKPFVHPKPPPPLPPSAPSLPLEPPRSTPPRSSLYPALT
PSLGAKPKPQVLSDSGGPLIDLLTEDPPPYRDPRPPPSDRDGNGG
EATPAGEAPDPSPMASRLRGRREPPVADSTTSQAFPLRAGGNGQL
QYWPFSSSDLYNWKNNNPSFSEDPGKLTALIESVLITHQPTWDDC
QQLLGTLLTGEEKQRVLLEARKAVRGDDGRPTQLPNEVDAAFPLE
RPDWDYTTQAGRNHLVHYRQLLLAGLQNAGRSPTNLAKVKGITQG
PNESPSAFLERLKEAYRRYTPYDPEDPGQETNVSMSFIWQSAPDI
GRKLERLEDLKNKTLGDLVREAEKIFNKRETPEEREERIRRETEE
KEERRRTEDEQKEKERDRRRHREMSKLLATVVSGQKQDRQGGERR
RSQLDRDQCAYCKEKGHWAKDCPKKPRGPRGPRPQTSLLTLDD

In some embodiments, the amino acid sequence of the gag protein comprising a viral nucleocapsid protein variant comprises one or more substitutions at positions selected from the group consisting of 215, 216, 218, 219, 220, 221, 222, 223, 224, 225, 226, 227, 228, 229, 230, 231, 232, 233, 234, 235, 236, 237, 238, 239, 240, 241, 242, 244, 245, 246, 249, 250, 253, 254, 255, 256, 257, 258, 259, 260, 261, 262, 263, 264, 265, 266, 269, 271, 272, 274, 275, 276, 277, 279, 280, 281, 282, 283, 285, 286, 288, 289, 291, 292, 293, 294, 295, 296, 297, 298, 299, 300, 301, 302, 303, 304, 305, 307, 308, 310, 311, 418, 420, 421, 424, 427, 430, 432, 433, 435, 436, 437, 438, 440, 441, 443, 444, 446, 447, 448, 449, 452, 455, 458, 460, 463, 464, 466, 467, 468, 469, 470, 471, 472, 473, 474, 475, 477, 478, 479, 480, 481, 482, 483, 485, 486, 487, 488, 489, 490, 491, 492, 495, 496, 497, 498, 499, 500, 501, 502, 503, 504, 505, 506, 507, 508, 509, 510, 511, and 512 relative to the amino acid sequence provided in SEQ ID NO: 100, or at corresponding positions in a homologous viral nucleocapsid protein.

In certain embodiments, the amino acid sequence of the gag protein comprising a viral nucleocapsid protein variant comprises one or more substitutions selected from the group consisting of F215L, F215G, F215M, P216K, P216I, R218I, R218G, R218H, A219C, A219K, A219N, A219G, G220C, G221W, G221M, N222H, N222I, N222M, N222Y, N222V, G223K, G223A, G223C, G223D, Q224L, Q224R, Q224F, Q224I, L225Q, Q226P, Y227S, W228N, W228F, P229D, P229L, F230C, F230E, S231Y, S23iF, S232L, S232A, S233K, S233I, S233R, D234E, D234A, L235C, L235M, Y236E, Y236M, N237D, W238D, K239A, K239T, K239H, N240A, N240E, N240L, N240S, N240I, N241K, N241V, N242T, S244Y, S244M, S244T, F245H, F245R, F245W, S246L, S246H, S246F, S246V, S246Y, P249S, P249F, P249K, G250C, G250D, G250L, G250R, T253F, A254W, L255H, L255V, I256V, I256W, E257A, E257C, S258W, S258V, V259R, L260M, L260W, L2601, 1261K, I261W, I261Q, T262N, T262W, T262F, T262Q, H263I, Q264S, Q264T, P265G, P265F, T266C, D269G, Q27iF, Q271A, Q271D, Q272P, Q272G, L274W, G275W, T276R, T276V, T276M, L277W, T279G, G280W, G280D, G280Y, E281T, E281N, E281C, E282S, E282H, E282Y, K283F, R285V, V286H, L288D, L288K, L288A, E289C, R291K, K292L, A293H, A293Y, V294Q, R295M, G296D, D297N, D297A, D297M, D297P, D297W, D298Y, G299M, G299R, G299Y, R300L, P301S, P301L, T302V, Q303S, L304N, P305R, P305G, P305M, E307N, V308R, V308I, A310R, A311F, A311E, A311M, K418Y, L420C, G421D, G421A, G421R, V424Y, A427G, I430W, I430H, N432Y, K433P, E435A, T436N, P437K, E438H, R440C, R440P, E441M, R443P, I444E, R446G, R446I, E447H, E447S, T448M, T448W, T448V, E449D, E452S, R455Q, E458F, E460W, E463H, K464L, R466Q, R466A, D467E, R468Q, R468W, R468I, R468S, R469Q, R470A, R470M, R470W, H471M, H471D, H471G, H47iN, R472E, R472D, E473N, M474W, M474C, M474I, S475G, L477W, L477C, L477E, L478K, L478V, L478P, A479Q, A479L, A479K, A479S, A479Y, T480H, T480W, T480E, V48iT, V482L, V482M, S483W, S483L, S483R, Q485K, Q485G, Q485L, Q485W, K486L, K486I, K486V, K486E, K486F, Q487P, Q487E, D488Q, D488I, R489A, R489K, R489M, R489F, Q490T, G491C, G491Q, G491H, G492Q, G492W, G492H, R495Y, R495K, R495F, R495I, R496L, R496N, R496F, R496A, R496K, S497C, S497P, S497Q, S497T, S497N, Q498K, Q498V, L499Y, L499F, L499G, L499T, D500Q, D500G, D500M, D500I, R501D, R501I, R501L, D502Q, D502A, D502P, Q503T, C504N, C504S, A505M, A505Y, A505W, A505I, Y506L, Y506M, C507F, C507V, C507G, C507W, K508D, K508N, E509G, E509A, E509M, K510M, K510P, K510R, G511L, G511K, G511P, H512Q, H512S, and H512M relative to the amino acid sequence provided in SEQ ID NO: 100, or corresponding substitutions in a homologous viral nucleocapsid protein.

In some embodiments, the amino acid sequence of the gag protein comprising a viral nucleocapsid protein variant comprises one or more substitutions at positions which result in a VLP comprising the variant being produced at higher levels and/or packaging a cargo molecule more efficiently, as compared to a VLP comprising a nucleocapsid protein that does not comprise these mutations. In some embodiments, the nucleocapsid protein variant comprises an amino acid substitution at position 222 relative to SEQ ID NO: 100. In certain embodiments, the amino acid substitution is an N222H substitution. In certain embodiments, the amino acid substitution is an N222I substitution. In some embodiments, the nucleocapsid protein variant comprises an amino acid substitution at position 226 relative to SEQ ID NO: 100. In certain embodiments, the amino acid substitution is a Q226P substitution. In some embodiments, the nucleocapsid protein variant comprises an amino acid substitution at position 227 relative to SEQ ID NO: 100. In certain embodiments, the amino acid substitution is a Y227S substitution. In some embodiments, the nucleocapsid protein variant comprises an amino acid substitution at position 228 relative to SEQ ID NO: 100. In certain embodiments, the amino acid substitution is a W228N substitution. In some embodiments, the nucleocapsid protein variant comprises an amino acid substitution at position 229 relative to SEQ ID NO: 100. In certain embodiments, the amino acid substitution is a P229D substitution. In some embodiments, the nucleocapsid protein variant comprises an amino acid substitution at position 230 relative to SEQ ID NO: 100. In certain embodiments, the amino acid substitution is an F230C substitution. In some embodiments, the nucleocapsid protein variant comprises an amino acid substitution at position 235 relative to SEQ ID NO: 100. In certain embodiments, the amino acid substitution is an L235C substitution. In some embodiments, the nucleocapsid protein variant comprises an amino acid substitution at position 239 relative to SEQ ID NO: 100. In certain embodiments, the amino acid substitution is a K239A substitution. In some embodiments, the nucleocapsid protein variant comprises an amino acid substitution at position 240 relative to SEQ ID NO: 100. In certain embodiments, the amino acid substitution is an N240A substitution. In some embodiments, the nucleocapsid protein variant comprises an amino acid substitution at position 244 relative to SEQ ID NO: 100. In certain embodiments, the amino acid substitution is an S244Y substitution. In some embodiments, the nucleocapsid protein variant comprises an amino acid substitution at position 245 relative to SEQ ID NO: 100. In certain embodiments, the amino acid substitution is an F245H substitution. In certain embodiments, the amino acid substitution is an F245R substitution. In some embodiments, the nucleocapsid protein variant comprises an amino acid substitution at position 246 relative to SEQ ID NO: 100. In certain embodiments, the amino acid substitution is an S246L substitution. In some embodiments, the nucleocapsid protein variant comprises an amino acid substitution at position 246 relative to SEQ ID NO: 100. In certain embodiments, the amino acid substitution is an S246H substitution. In some embodiments, the nucleocapsid protein variant comprises an amino acid substitution at position 253 relative to SEQ ID NO: 100. In certain embodiments, the amino acid substitution is a T253F substitution. In some embodiments, the nucleocapsid protein variant comprises an amino acid substitution at position 254 relative to SEQ ID NO: 100. In certain embodiments, the amino acid substitution is an A254W substitution. In some embodiments, the nucleocapsid protein variant comprises an amino acid substitution at position 256 relative to SEQ ID NO: 100. In certain embodiments, the amino acid substitution is an I256V substitution. In certain embodiments, the amino acid substitution is an I256W substitution. In some embodiments, the nucleocapsid protein variant comprises an amino acid substitution at position 260 relative to SEQ ID NO: 100. In certain embodiments, the amino acid substitution is an L260M substitution. In certain embodiments, the amino acid substitution is an L260I substitution. In some embodiments, the nucleocapsid protein variant comprises an amino acid substitution at position 261 relative to SEQ ID NO: 100. In certain embodiments, the amino acid substitution is an I261K substitution. In some embodiments, the nucleocapsid protein variant comprises an amino acid substitution at position 272 relative to SEQ ID NO: 100. In certain embodiments, the amino acid substitution is a Q272P substitution. In some embodiments, the nucleocapsid protein variant comprises an amino acid substitution at position 276 relative to SEQ ID NO: 100. In certain embodiments, the amino acid substitution is a T276R substitution. In some embodiments, the nucleocapsid protein variant comprises an amino acid substitution at position 277 relative to SEQ ID NO: 100. In certain embodiments, the amino acid substitution is an L277W substitution. In some embodiments, the nucleocapsid protein variant comprises an amino acid substitution at position 279 relative to SEQ ID NO: 100. In certain embodiments, the amino acid substitution is a T279G substitution. In some embodiments, the nucleocapsid protein variant comprises an amino acid substitution at position 292 relative to SEQ ID NO: 100. In certain embodiments, the amino acid substitution is a K292L substitution. In some embodiments, the nucleocapsid protein variant comprises an amino acid substitution at position 293 relative to SEQ ID NO: 100. In certain embodiments, the amino acid substitution is an A293H substitution. In some embodiments, the nucleocapsid protein variant comprises an amino acid substitution at position 297 relative to SEQ ID NO: 100. In certain embodiments, the amino acid substitution is a D297N substitution. In certain embodiments, the amino acid substitution is a D297A substitution. In some embodiments, the nucleocapsid protein variant comprises an amino acid substitution at position 305 relative to SEQ ID NO: 100. In certain embodiments, the amino acid substitution is a P305R substitution. In some embodiments, the nucleocapsid protein variant comprises an amino acid substitution at position 308 relative to SEQ ID NO: 100. In certain embodiments, the amino acid substitution is a V308R substitution. In some embodiments, the nucleocapsid protein variant comprises an amino acid substitution at position 418 relative to SEQ ID NO: 100. In certain embodiments, the amino acid substitution is a K418Y substitution. In some embodiments, the nucleocapsid protein variant comprises an amino acid substitution at position 427 relative to SEQ ID NO: 100. In certain embodiments, the amino acid substitution is an A427G substitution. In some embodiments, the nucleocapsid protein variant comprises an amino acid substitution at position 463 relative to SEQ ID NO: 100. In certain embodiments, the amino acid substitution is an E463H substitution. In some embodiments, the nucleocapsid protein variant comprises an amino acid substitution at position 466 relative to SEQ ID NO: 100. In certain embodiments, the amino acid substitution is an R466Q substitution. In some embodiments, the nucleocapsid protein variant comprises an amino acid substitution at position 467 relative to SEQ ID NO: 100. In certain embodiments, the amino acid substitution is a D467E substitution. In some embodiments, the nucleocapsid protein variant comprises an amino acid substitution at position 470 relative to SEQ ID NO: 100. In certain embodiments, the amino acid substitution is an R470A substitution. In some embodiments, the nucleocapsid protein variant comprises an amino acid substitution at position 471 relative to SEQ ID NO: 100. In certain embodiments, the amino acid substitution is an H471M substitution. In some embodiments, the nucleocapsid protein variant comprises an amino acid substitution at position 472 relative to SEQ ID NO: 100. In certain embodiments, the amino acid substitution is an R472E substitution. In some embodiments, the nucleocapsid protein variant comprises an amino acid substitution at position 473 relative to SEQ ID NO: 100. In certain embodiments, the amino acid substitution is an E473N substitution. In some embodiments, the nucleocapsid protein variant comprises an amino acid substitution at position 477 relative to SEQ ID NO: 100. In certain embodiments, the amino acid substitution is an L477W substitution. In some embodiments, the nucleocapsid protein variant comprises an amino acid substitution at position 478 relative to SEQ ID NO: 100. In certain embodiments, the amino acid substitution is an L478K substitution. In some embodiments, the nucleocapsid protein variant comprises an amino acid substitution at position 479 relative to SEQ ID NO: 100. In certain embodiments, the amino acid substitution is an A479Q substitution. In certain embodiments, the amino acid substitution is an A479L substitution. In some embodiments, the nucleocapsid protein variant comprises an amino acid substitution at position 482 relative to SEQ ID NO: 100. In certain embodiments, the amino acid substitution is a V482M substitution. In some embodiments, the nucleocapsid protein variant comprises an amino acid substitution at position 483 relative to SEQ ID NO: 100. In certain embodiments, the amino acid substitution is an S483W substitution. In some embodiments, the nucleocapsid protein variant comprises an amino acid substitution at position 485 relative to SEQ ID NO: 100. In certain embodiments, the amino acid substitution is a Q485K substitution. In some embodiments, the nucleocapsid protein variant comprises an amino acid substitution at position 496 relative to SEQ ID NO: 100. In certain embodiments, the amino acid substitution is an R496L substitution. In some embodiments, the nucleocapsid protein variant comprises an amino acid substitution at position 497 relative to SEQ ID NO: 100. In certain embodiments, the amino acid substitution is an S497C substitution. In some embodiments, the nucleocapsid protein variant comprises an amino acid substitution at position 499 relative to SEQ ID NO: 100. In certain embodiments, the amino acid substitution is an L499Y substitution. In certain embodiments, the amino acid substitution is an L499F substitution. In some embodiments, the nucleocapsid protein variant comprises an amino acid substitution at position 500 relative to SEQ ID NO: 100. In certain embodiments, the amino acid substitution is a D500Q substitution. In some embodiments, the nucleocapsid protein variant comprises an amino acid substitution at position 505 relative to SEQ ID NO: 100. In certain embodiments, the amino acid substitution is an A505M substitution. In some embodiments, the nucleocapsid protein variant comprises an amino acid substitution at position 506 relative to SEQ ID NO: 100. In certain embodiments, the amino acid substitution is a Y506L substitution. In some embodiments, the nucleocapsid protein variant comprises an amino acid substitution at position 507 relative to SEQ ID NO: 100. In certain embodiments, the amino acid substitution is a C507F substitution.

In some embodiments, the amino acid sequence of the gag protein comprising a viral nucleocapsid protein variant comprises one or more substitutions at positions which result in a VLP comprising the variant having a higher transduction efficiency into target cells, as compared to a VLP comprising a nucleocapsid protein that does not comprise these mutations. In some embodiments, the nucleocapsid protein variant comprises an amino acid substitution at position 215 relative to SEQ ID NO: 100. In certain embodiments, the amino acid substitution is an F215G substitution. In some embodiments, the nucleocapsid protein variant comprises an amino acid substitution at position 219 relative to SEQ ID NO: 100. In certain embodiments, the amino acid substitution is an A219C substitution. In some embodiments, the nucleocapsid protein variant comprises an amino acid substitution at position 226 relative to SEQ ID NO: 100. In certain embodiments, the amino acid substitution is a Q226P substitution. In some embodiments, the nucleocapsid protein variant comprises an amino acid substitution at position 257 relative to SEQ ID NO: 100. In certain embodiments, the amino acid substitution is an E257C substitution. In some embodiments, the nucleocapsid protein variant comprises an amino acid substitution at position 261 relative to SEQ ID NO: 100. In certain embodiments, the amino acid substitution is an I261W substitution. In some embodiments, the nucleocapsid protein variant comprises an amino acid substitution at position 272 relative to SEQ ID NO: 100. In certain embodiments, the amino acid substitution is a Q272G substitution. In some embodiments, the nucleocapsid protein variant comprises an amino acid substitution at position 280 relative to SEQ ID NO: 100. In certain embodiments, the amino acid substitution is a G280W substitution. In some embodiments, the nucleocapsid protein variant comprises an amino acid substitution at position 283 relative to SEQ ID NO: 100. In certain embodiments, the amino acid substitution is a K283F substitution. In some embodiments, the nucleocapsid protein variant comprises an amino acid substitution at position 288 relative to SEQ ID NO: 100. In certain embodiments, the amino acid substitution is an L288A substitution. In some embodiments, the nucleocapsid protein variant comprises an amino acid substitution at position 293 relative to SEQ ID NO: 100. In certain embodiments, the amino acid substitution is an A293Y substitution. In some embodiments, the nucleocapsid protein variant comprises an amino acid substitution at position 310 relative to SEQ ID NO: 100. In certain embodiments, the amino acid substitution is an A310R substitution. In some embodiments, the nucleocapsid protein variant comprises an amino acid substitution at position 467 relative to SEQ ID NO: 100. In certain embodiments, the amino acid substitution is a D467E substitution. In some embodiments, the nucleocapsid protein variant comprises an amino acid substitution at position 469 relative to SEQ ID NO: 100. In certain embodiments, the amino acid substitution is an R469Q substitution. In some embodiments, the nucleocapsid protein variant comprises an amino acid substitution at position 471 relative to SEQ ID NO: 100. In certain embodiments, the amino acid substitution is an H471D substitution. In certain embodiments, the amino acid substitution is an H471M substitution. In some embodiments, the nucleocapsid protein variant comprises an amino acid substitution at position 472 relative to SEQ ID NO: 100. In certain embodiments, the amino acid substitution is an R472E substitution. In some embodiments, the nucleocapsid protein variant comprises an amino acid substitution at position 479 relative to SEQ ID NO: 100. In certain embodiments, the amino acid substitution is an A479K substitution. In some embodiments, the nucleocapsid protein variant comprises an amino acid substitution at position 480 relative to SEQ ID NO: 100. In certain embodiments, the amino acid substitution is a T480H substitution. In some embodiments, the nucleocapsid protein variant comprises an amino acid substitution at position 482 relative to SEQ ID NO: 100. In certain embodiments, the amino acid substitution is a V482M substitution. In some embodiments, the nucleocapsid protein variant comprises an amino acid substitution at position 485 relative to SEQ ID NO: 100. In certain embodiments, the amino acid substitution is a Q485K substitution. In certain embodiments, the amino acid substitution is a Q485L substitution. In some embodiments, the nucleocapsid protein variant comprises an amino acid substitution at position 490 relative to SEQ ID NO: 100. In certain embodiments, the amino acid substitution is a Q490T substitution. In some embodiments, the nucleocapsid protein variant comprises an amino acid substitution at position 492 relative to SEQ ID NO: 100. In certain embodiments, the amino acid substitution is a G492W substitution. In some embodiments, the nucleocapsid protein variant comprises an amino acid substitution at position 496 relative to SEQ ID NO: 100. In certain embodiments, the amino acid substitution is an R496F substitution. In some embodiments, the nucleocapsid protein variant comprises an amino acid substitution at position 497 relative to SEQ ID NO: 100. In certain embodiments, the amino acid substitution is an S497Q substitution. In some embodiments, the nucleocapsid protein variant comprises an amino acid substitution at position 500 relative to SEQ ID NO: 100. In certain embodiments, the amino acid substitution is a D500Q substitution. In some embodiments, the nucleocapsid protein variant comprises an amino acid substitution at position 501 relative to SEQ ID NO: 100. In certain embodiments, the amino acid substitution is an R501D substitution. In certain embodiments, the amino acid substitution is an R501I substitution. In some embodiments, the nucleocapsid protein variant comprises an amino acid substitution at position 502 relative to SEQ ID NO: 100. In certain embodiments, the amino acid substitution is a D502Q substitution. In some embodiments, the nucleocapsid protein variant comprises an amino acid substitution at position 504 relative to SEQ ID NO: 100. In certain embodiments, the amino acid substitution is a C504N substitution. In some embodiments, the nucleocapsid protein variant comprises an amino acid substitution at position 505 relative to SEQ ID NO: 100. In certain embodiments, the amino acid substitution is an A505M substitution. In certain embodiments, the amino acid substitution is an A505W substitution. In some embodiments, the nucleocapsid protein variant comprises an amino acid substitution at position 507 relative to SEQ ID NO: 100. In certain embodiments, the amino acid substitution is a C507V substitution. In certain embodiments, the amino acid substitution is a C507F substitution. In some embodiments, the nucleocapsid protein variant comprises an amino acid substitution at position 508 relative to SEQ ID NO: 100. In certain embodiments, the amino acid substitution is a K508D substitution.

In some embodiments, the present disclosure comprises gag proteins comprising one or more, two or more, three or more, four or more, or five or more substitutions at positions selected from the group consisting of 215, 219, 226, 233, 255, 256, 260, 261, 272, 280, 283, 288, 310, 440, 443, 444, 469, 471, 472, 478, 479, 480, 481, 485, 490, 492, 496, 497, 500, 501, 502, 505, and 507, optionally wherein when incorporated into a VLP the gag protein facilitates higher packaging of one or more cargo molecules in the VLP and/or increased production of the VLP in a producer cell, and/or optionally wherein when incorporated into a VLP the gag protein facilitates improved transduction efficiency of the VLP into a target cell type. In certain embodiments, the gag protein comprises one or more, two or more, three or more, four or more, or five or more of the amino acid substitutions F215G, A219C, Q226P, S233K, L255V, 1256W, L2601, 1261W, Q272G, G280W, K283F, L288A, A310R, R440P, R443P, 1444E, R469Q, H471D, H471M, R472E, L478K, A479K, T480H, V48iT, Q485L, Q490T, G492W, R496F, S497Q, D500Q, R501I, D502Q, A505M, A505W, C507F, and C507V, optionally wherein when incorporated into a VLP the gag protein facilitates higher packaging of one or more cargo molecules in the VLP and/or increased production of the VLP in a producer cell, and/or optionally wherein when incorporated into a VLP the gag protein facilitates improved transduction efficiency of the VLP into a target cell type. In certain embodiments, the gag protein comprises one or more, two or more, three or more, four or more, or five or more of the amino acid substitutions F215G, Q226P, L260I, G280W, A310R, 1444E, L478K, A479K, T480H, V481T, Q490T, G492W, A505W, and C507V, wherein when incorporated into a VLP the gag protein facilitates higher packaging of one or more cargo molecules in the VLP and/or increased production of the VLP in a producer cell. In certain embodiments, the gag protein comprises one or more, two or more, three or more, four or more, or five or more of the amino acid substitutions L255V, I256W, L260I, L288A, R440P, and R443P, wherein when incorporated into a VLP the gag protein facilitates improved transduction efficiency of the VLP into a target cell type.

In some embodiments, the nucleocapsid protein variant comprises amino acid substitutions at one or more positions selected from the group consisting of 226, 501, 502, 504, 505, 507, and 508 relative to the amino acid sequence provided in SEQ ID NO: 100, or at corresponding positions in a homologous viral nucleocapsid protein. In certain embodiments, the nucleocapsid protein variant comprises one or more amino acid substitutions selected from the group consisting of Q226P, R501D, D502Q, C504N, A505W, C507F, C507V, and K508D relative to the amino acid sequence provided in SEQ ID NO: 100, or corresponding substitutions in a homologous viral nucleocapsid protein. In some embodiments, the nucleocapsid protein variant comprises amino acid substitutions at two positions selected from the group consisting of 226, 501, 502, 504, 505, 507, and 508 relative to the amino acid sequence provided in SEQ ID NO: 100, or at corresponding positions in a homologous viral nucleocapsid protein. In certain embodiments, the nucleocapsid protein variant comprises two amino acid substitutions selected from the group consisting of Q226P, R501D, D502Q, C504N, A505W, C507F, C507V, and K508D relative to the amino acid sequence provided in SEQ ID NO: 100, or corresponding substitutions in a homologous viral nucleocapsid protein. In some embodiments, the nucleocapsid protein variant comprises an amino acid substitution at position 226 relative to the amino acid sequence provided in SEQ ID NO: 100, or at a corresponding position in a homologous viral nucleocapsid protein. In certain embodiments, the nucleocapsid protein variant comprises the amino acid substitution Q226P relative to the amino acid sequence provided in SEQ ID NO: 100, or a corresponding substitution in a homologous viral nucleocapsid protein. In some embodiments, the nucleocapsid protein variant comprises an amino acid substitution at position 501 relative to the amino acid sequence provided in SEQ ID NO: 100, or at a corresponding position in a homologous viral nucleocapsid protein. In certain embodiments, the nucleocapsid protein variant comprises the amino acid substitution R501D relative to the amino acid sequence provided in SEQ ID NO: 100, or a corresponding substitution in a homologous viral nucleocapsid protein. In certain embodiments, the nucleocapsid protein variant comprises the amino acid substitution R501I relative to the amino acid sequence provided in SEQ ID NO: 100, or a corresponding substitution in a homologous viral nucleocapsid protein. In some embodiments, the nucleocapsid protein variant comprises amino acid substitutions at positions 226 and 501 relative to the amino acid sequence provided in SEQ ID NO: 100, or at corresponding positions in a homologous viral nucleocapsid protein. In certain embodiments, the nucleocapsid protein variant comprises the amino acid substitutions Q226P and R501D relative to the amino acid sequence provided in SEQ ID NO: 100, or corresponding substitutions in a homologous viral nucleocapsid protein. In certain embodiments, the nucleocapsid protein variant comprises the amino acid substitutions Q226P and R501I relative to the amino acid sequence provided in SEQ ID NO: 100, or corresponding substitutions in a homologous viral nucleocapsid protein. In some embodiments, the nucleocapsid protein variant comprises an amino acid substitution at position 507 relative to the amino acid sequence provided in SEQ ID NO: 100, or at a corresponding position in a homologous viral nucleocapsid protein. In certain embodiments, the nucleocapsid protein variant comprises the amino acid substitution C507V relative to the amino acid sequence provided in SEQ ID NO: 100, or a corresponding substitution in a homologous viral nucleocapsid protein.

In certain embodiments, the present disclosure provides a nucleocapsid protein variant comprising the amino acid sequence of SEQ ID NO: 105, or an amino acid sequence at least 80%, at least 85%, at least 90%, at least 95%, at least 96%, at least 97%, at least 98%, or at least 99% identical to the amino acid sequence of SEQ ID NO: 105:

(SEQ ID NO: 105)
MGQTVTTPLSLTLGHWKDVERIAHNQSVDVKKRRWVTFCSAEWPT
FNVGWPRDGTFNRDLITQVKIKVFSPGPHGHPDQVPYIVTWEALA
FDPPPWVKPFVHPKPPPPLPPSAPSLPLEPPRSTPPRSSLYPALT
PSLGAKPKPQVLSDSGGPLIDLLTEDPPPYRDPRPPPSDRDGNGG
EATPAGEAPDPSPMASRLRGRREPPVADSTTSQAFPLRAGGNGQL
PYWPFSSSDLYNWKNNNPSFSEDPGKLTALIESVLITHQPTWDDC
QQLLGTLLTGEEKQRVLLEARKAVRGDDGRPTQLPNEVDAAFPLE
RPDWDYTTQAGRNHLVHYRQLLLAGLQNAGRSPTNLAKVKGITQG
PNESPSAFLERLKEAYRRYTPYDPEDPGQETNVSMSFIWQSAPDI
GRKLERLEDLKNKTLGDLVREAEKIFNKRETPEEREERIRRETEE
KEERRRTEDEQKEKERDRRRHREMSKLLATVVSGQKQDRQGGERR
RSQLDRDQCAYCKEKGHWAKDCPKKPRGPRGPRPQTSLLTLDD.

In certain embodiments, the present disclosure provides a nucleocapsid protein variant comprising the amino acid sequence of SEQ ID NO: 106, or an amino acid sequence at least 80%, at least 85%, at least 90%, at least 95%, at least 96%, at least 97%, at least 98%, or at least 99% identical to the amino acid sequence of SEQ ID NO: 106:

(SEQ ID NO: 106)
MGQTVTTPLSLTLGHWKDVERIAHNQSVDVKKRRWVTFCSAEWPT
FNVGWPRDGTFNRDLITQVKIKVFSPGPHGHPDQVPYIVTWEALA
FDPPPWVKPFVHPKPPPPLPPSAPSLPLEPPRSTPPRSSLYPALT
PSLGAKPKPQVLSDSGGPLIDLLTEDPPPYRDPRPPPSDRDGNGG
EATPAGEAPDPSPMASRLRGRREPPVADSTTSQAFPLRAGGNGQL
QYWPFSSSDLYNWKNNNPSFSEDPGKLTALIESVLITHQPTWDDC
QQLLGTLLTGEEKQRVLLEARKAVRGDDGRPTQLPNEVDAAFPLE
RPDWDYTTQAGRNHLVHYRQLLLAGLQNAGRSPTNLAKVKGITQG
PNESPSAFLERLKEAYRRYTPYDPEDPGQETNVSMSFIWQSAPDI
GRKLERLEDLKNKTLGDLVREAEKIFNKRETPEEREERIRRETEE
KEERRRTEDEQKEKERDRRRHREMSKLLATVVSGQKQDRQGGERR
RSQLDDDQCAYCKEKGHWAKDCPKKPRGPRGPRPQTSLLTLDD.

In certain embodiments, the present disclosure provides a nucleocapsid protein variant comprising the amino acid sequence of SEQ ID NO: 107, or an amino acid sequence at least 80%, at least 85%, at least 90%, at least 95%, at least 96%, at least 97%, at least 98%, or at least 99% identical to the amino acid sequence of SEQ ID NO: 107:

(SEQ ID NO: 107)
MGQTVTTPLSLTLGHWKDVERIAHNQSVDVKKRRWVTFCSAEWPT
FNVGWPRDGTFNRDLITQVKIKVFSPGPHGHPDQVPYIVTWEALA
FDPPPWVKPFVHPKPPPPLPPSAPSLPLEPPRSTPPRSSLYPALT
PSLGAKPKPQVLSDSGGPLIDLLTEDPPPYRDPRPPPSDRDGNGG
EATPAGEAPDPSPMASRLRGRREPPVADSTTSQAFPLRAGGNGQL
QYWPFSSSDLYNWKNNNPSFSEDPGKLTALIESVLITHQPTWDDC
QQLLGTLLTGEEKQRVLLEARKAVRGDDGRPTQLPNEVDAAFPLE
RPDWDYTTQAGRNHLVHYRQLLLAGLQNAGRSPTNLAKVKGITQG
PNESPSAFLERLKEAYRRYTPYDPEDPGQETNVSMSFIWQSAPDI
GRKLERLEDLKNKTLGDLVREAEKIFNKRETPEEREERIRRETEE
KEERRRTEDEQKEKERDRRRHREMSKLLATVVSGQKQDRQGGERR
RSQLDIDQCAYCKEKGHWAKDCPKKPRGPRGPRPQTSLLTLDD.

In certain embodiments, the present disclosure provides a nucleocapsid protein variant comprising the amino acid sequence of SEQ ID NO: 108, or an amino acid sequence at least 80%, at least 85%, at least 90%, at least 95%, at least 96%, at least 97%, at least 98%, or at least 99% identical to the amino acid sequence of SEQ ID NO: 108:

(SEQ ID NO: 108)
MGQTVTTPLSLTLGHWKDVERIAHNQSVDVKKRRWVTFCSAEWPT
FNVGWPRDGTFNRDLITQVKIKVFSPGPHGHPDQVPYIVTWEALA
FDPPPWVKPFVHPKPPPPLPPSAPSLPLEPPRSTPPRSSLYPALT
PSLGAKPKPQVLSDSGGPLIDLLTEDPPPYRDPRPPPSDRDGNGG
EATPAGEAPDPSPMASRLRGRREPPVADSTTSQAFPLRAGGNGQL
QYWPFSSSDLYNWKNNNPSFSEDPGKLTALIESVLITHQPTWDDC
QQLLGTLLTGEEKQRVLLEARKAVRGDDGRPTQLPNEVDAAFPLE
RPDWDYTTQAGRNHLVHYRQLLLAGLQNAGRSPTNLAKVKGITQG
PNESPSAFLERLKEAYRRYTPYDPEDPGQETNVSMSFIWQSAPDI
GRKLERLEDLKNKTLGDLVREAEKIFNKRETPEEREERIRRETEE
KEERRRTEDEQKEKERDRRRHREMSKLLATVVSGQKQDRQGGERR
RSQLDRDQCAYVKEKGHWAKDCPKKPRGPRGPRPQTSLLTLDD.

In certain embodiments, the present disclosure provides a nucleocapsid protein variant comprising the amino acid sequence of SEQ ID NO: 109, or an amino acid sequence at least 80%, at least 85%, at least 90%, at least 95%, at least 96%, at least 97%, at least 98%, or at least 99% identical to the amino acid sequence of SEQ ID NO: 109:

(SEQ ID NO: 109)
MGQTVTTPLSLTLGHWKDVERIAHNQSVDVKKRRWVTFCSAEWPT
FNVGWPRDGTFNRDLITQVKIKVFSPGPHGHPDQVPYIVTWEALA
FDPPPWVKPFVHPKPPPPLPPSAPSLPLEPPRSTPPRSSLYPALT
PSLGAKPKPQVLSDSGGPLIDLLTEDPPPYRDPRPPPSDRDGNGG
EATPAGEAPDPSPMASRLRGRREPPVADSTTSQAFPLRAGGNGQL
PYWPFSSSDLYNWKNNNPSFSEDPGKLTALIESVLITHQPTWDDC
QQLLGTLLTGEEKQRVLLEARKAVRGDDGRPTQLPNEVDAAFPLE
RPDWDYTTQAGRNHLVHYRQLLLAGLQNAGRSPTNLAKVKGITQG
PNESPSAFLERLKEAYRRYTPYDPEDPGQETNVSMSFIWQSAPDI
GRKLERLEDLKNKTLGDLVREAEKIFNKRETPEEREERIRRETEE
KEERRRTEDEQKEKERDRRRHREMSKLLATVVSGQKQDRQGGERR
RSQLDDDQCAYCKEKGHWAKDCPKKPRGPRGPRPQTSLLTLDD.

In certain embodiments, the present disclosure provides a nucleocapsid protein variant comprising the amino acid sequence of SEQ ID NO: 110, or an amino acid sequence at least 80%, at least 85%, at least 90%, at least 95%, at least 96%, at least 97%, at least 98%, or at least 99% identical to the amino acid sequence of SEQ ID NO: 110:

(SEQ ID NO: 110)
MGQTVTTPLSLTLGHWKDVERIAHNQSVDVKKRRWVTFCSAEWPT
FNVGWPRDGTFNRDLITQVKIKVFSPGPHGHPDQVPYIVTWEALA
FDPPPWVKPFVHPKPPPPLPPSAPSLPLEPPRSTPPRSSLYPALT
PSLGAKPKPQVLSDSGGPLIDLLTEDPPPYRDPRPPPSDRDGNGG
EATPAGEAPDPSPMASRLRGRREPPVADSTTSQAFPLRAGGNGQL
PYWPFSSSDLYNWKNNNPSFSEDPGKLTALIESVLITHQPTWDDC
QQLLGTLLTGEEKQRVLLEARKAVRGDDGRPTQLPNEVDAAFPLE
RPDWDYTTQAGRNHLVHYRQLLLAGLQNAGRSPTNLAKVKGITQG
PNESPSAFLERLKEAYRRYTPYDPEDPGQETNVSMSFIWQSAPDI
GRKLERLEDLKNKTLGDLVREAEKIFNKRETPEEREERIRRETEE
KEERRRTEDEQKEKERDRRRHREMSKLLATVVSGQKQDRQGGERR
RSQLDIDQCAYCKEKGHWAKDCPKKPRGPRGPRPQTSLLTLDD.

In another aspect, the present disclosure provides VLPs comprising any of the gag proteins comprising a nucleocapsid protein variant provided herein. Such VLPs may also comprise envelope glycoproteins, gag-pro-polyproteins, cargo molecules, and any other components as described herein. In some embodiments, the VLP comprises a gag-pro polyprotein and a fusion protein encapsulated by a viral envelope glycoprotein, wherein the fusion protein comprises a gag protein comprising any of the nucleocapsid protein variants disclosed herein, a cargo, a cleavable linker, and a nuclear export sequence (NES). In some embodiments, the VLP comprises a gag-pro polyprotein and a fusion protein encapsulated by a viral envelope glycoprotein, wherein the gag-pro polyprotein comprises a gag protein comprising any of the nucleocapsid protein variants disclosed herein, and wherein the fusion protein comprises a gag protein, a cargo, a cleavable linker, and a nuclear export sequence (NES). In some embodiments, the VLP comprises a gag-pro polyprotein and a fusion protein encapsulated by a viral envelope glycoprotein, wherein the gag-pro polyprotein comprises a gag protein comprising any of the nucleocapsid protein variants disclosed herein, and wherein the fusion protein comprises a gag protein comprising any of the nucleocapsid protein variants disclosed herein, a cargo, a cleavable linker, and a nuclear export sequence (NES).

In some embodiments, the present disclosure provides VLPs wherein the gag protein of either the gag-pro polyprotein and/or the gag fusion protein each comprise one or more substitutions at amino acid positions selected from the group consisting of 226, 501, 502, 504, 505, 507, and 508 relative to the amino acid sequence provided in SEQ ID NO: 100, or at corresponding positions in a homologous viral nucleocapsid protein. In certain embodiments, the gag protein of either the gag-pro polyprotein and/or the gag fusion protein each comprise one or more substitutions selected from the group consisting of Q226P, R501D, D502Q, C504N, A505W, C507F, C507V, and K508D relative to the amino acid sequence provided in SEQ ID NO: 100, or corresponding substitutions in a homologous viral nucleocapsid protein. In some embodiments, each of the gag proteins of the gag-pro polyprotein and the gag fusion protein independently comprise one, two, three, four, or five or more amino acid substitutions relative to a wild type protein. In certain embodiments, each of the gag proteins of the gag-pro polyprotein and the gag fusion protein comprise one amino acid substitution relative to a wild type protein. In certain embodiments, each of the gag proteins of the gag-pro polyprotein and the gag fusion protein comprise two amino acid substitutions relative to a wild type protein.

The VLPs provided herein may comprise the same nucleocapsid protein variant in the gag proteins of both the gag-pro polyprotein and the gag fusion protein. The VLPs provided herein may also comprise different nucleocapsid protein variants in the gag proteins of the gag-pro polyprotein and the gag fusion protein. In some embodiments, the present disclosure provides VLPs wherein the gag protein of either the gag-pro polyprotein and/or the gag fusion protein each comprise a substitution at amino acid position 226 relative to the amino acid sequence provided in SEQ ID NO: 100, or at a corresponding position in a homologous viral nucleocapsid protein; and wherein the gag protein of either the gag-pro polyprotein and/or the gag fusion protein each comprise a substitution at an amino acid position selected from the group consisting of 501, 502, 504, 505, 507, and 508 relative to the amino acid sequence provided in SEQ ID NO: 100, or at corresponding positions in a homologous viral nucleocapsid protein. In certain embodiments, the present disclosure provides VLPs wherein the gag protein of either the gag-pro polyprotein and/or the gag fusion protein each comprise the substitution Q226P relative to the amino acid sequence provided in SEQ ID NO: 100, or a corresponding substitution in a homologous viral nucleocapsid protein; and wherein the gag protein of either the gag-pro polyprotein and/or the gag fusion protein each comprise a substitution at an amino acid position selected from the group consisting of R501D, D502Q, C504N, A505W, C507F, C507V, and K508D relative to the amino acid sequence provided in SEQ ID NO: 100, or corresponding substitutions in a homologous viral nucleocapsid protein.

In some embodiments, the present disclosure provides VLPs wherein the gag protein of the gag-pro polyprotein comprises a substitution at amino acid position 226 relative to the amino acid sequence provided in SEQ ID NO: 100, or at a corresponding position in a homologous viral nucleocapsid protein; and wherein the gag protein of the gag fusion protein comprises a substitution at amino acid position 501 relative to the amino acid sequence provided in SEQ ID NO: 100, or at a corresponding position in a homologous viral nucleocapsid protein. In certain embodiments, the present disclosure provides VLPs wherein the gag protein of the gag-pro polyprotein comprises the substitution Q226P relative to the amino acid sequence provided in SEQ ID NO: 100, or a corresponding substitution in a homologous viral nucleocapsid protein; and wherein the gag protein of the gag fusion protein comprises the substitution R501D relative to the amino acid sequence provided in SEQ ID NO: 100, or a corresponding substitution in a homologous viral nucleocapsid protein.

In some embodiments, the present disclosure provides VLPs wherein the gag protein of the gag-pro polyprotein comprises a substitution at amino acid position 501 relative to the amino acid sequence provided in SEQ ID NO: 100, or at a corresponding position in a homologous viral nucleocapsid protein; and wherein the gag protein of the gag fusion protein comprises substitutions at amino acid positions 226 and 501 relative to the amino acid sequence provided in SEQ ID NO: 100, or at corresponding positions in a homologous viral nucleocapsid protein. In certain embodiments, the present disclosure provides VLPs wherein the gag protein of the gag-pro polyprotein comprises the substitution R501I relative to the amino acid sequence provided in SEQ ID NO: 100, or a corresponding substitution in a homologous viral nucleocapsid protein; and wherein the gag protein of the gag fusion protein comprises the substitutions Q226P and R501I relative to the amino acid sequence provided in SEQ ID NO: 100, or corresponding substitutions in a homologous viral nucleocapsid protein.

In some embodiments, the present disclosure provides VLPs wherein the gag protein of the gag-pro polyprotein comprises a substitution at amino acid position 226 relative to the amino acid sequence provided in SEQ ID NO: 100, or at a corresponding position in a homologous viral nucleocapsid protein; and wherein the gag protein of the gag fusion protein comprises a substitution at amino acid position 507 relative to the amino acid sequence provided in SEQ ID NO: 100, or at a corresponding position in a homologous viral nucleocapsid protein. In certain embodiments, the present disclosure provides VLPs wherein the gag protein of the gag-pro polyprotein comprises the substitution Q226P relative to the amino acid sequence provided in SEQ ID NO: 100, or a corresponding substitution in a homologous viral nucleocapsid protein; and wherein the gag protein of the gag fusion protein comprises the substitution C507V relative to the amino acid sequence provided in SEQ ID NO: 100, or a corresponding substitution in a homologous viral nucleocapsid protein.

It should be appreciated that any of the amino acid mutations described herein, (e.g., Q226P) from a first amino acid residue (e.g., Q) to a second amino acid residue (e.g., P) may also include mutations from the first amino acid residue to an amino acid residue that is similar to (e.g., conserved) the second amino acid residue. For example, mutation of an amino acid with a hydrophobic side chain (e.g., alanine, valine, isoleucine, leucine, methionine, phenylalanine, tyrosine, or tryptophan) may be a mutation to a second amino acid with a different hydrophobic side chain (e.g., alanine, valine, isoleucine, leucine, methionine, phenylalanine, tyrosine, or tryptophan). For example, a mutation of an alanine to a threonine may also be a mutation from an alanine to an amino acid that is similar in size and chemical properties to a threonine, for example, serine. As another example, mutation of an amino acid with a positively charged side chain (e.g., arginine, histidine, or lysine) may be a mutation to a second amino acid with a different positively charged side chain (e.g., arginine, histidine, or lysine). As another example, mutation of an amino acid with a polar side chain (e.g., serine, threonine, asparagine, or glutamine) may be a mutation to a second amino acid with a different polar side chain (e.g., serine, threonine, asparagine, or glutamine). Additional similar amino acid pairs include, but are not limited to, the following: phenylalanine and tyrosine; asparagine and glutamine; methionine and cysteine; aspartic acid and glutamic acid; and arginine and lysine.

The skilled artisan would recognize that such conservative amino acid substitutions will likely have minor effects on protein structure and are likely to be well tolerated without compromising function. In some embodiments, any of the amino acid mutations provided herein from one amino acid to a threonine may be an amino acid mutation to a serine. In some embodiments, any of the amino acid mutations provided herein from one amino acid to an arginine may be an amino acid mutation to a lysine. In some embodiments, any of the amino acid mutations provided herein from one amino acid to an isoleucine may be an amino acid mutation to an alanine, valine, methionine, or leucine. In some embodiments, any of the amino acid mutations provided herein from one amino acid to a lysine may be an amino acid mutation to an arginine. In some embodiments, any of the amino acid mutations provided herein from one amino acid to an aspartic acid may be an amino acid mutation to a glutamic acid or asparagine. In some embodiments, any of the amino acid mutations provided herein from one amino acid to a valine may be an amino acid mutation to an alanine, isoleucine, methionine, or leucine. In some embodiments, any of the amino acid mutations provided herein from one amino acid to a glycine may be an amino acid mutation to an alanine. It should be appreciated, however, that additional conserved amino acid residues would be recognized by the skilled artisan, and any of the amino acid mutations to other conserved amino acid residues are also within the scope of this disclosure.

### Pharmaceutical Compositions

Other aspects of the present disclosure relate to pharmaceutical compositions comprising any of the VLPs, nucleocapsid protein variants, polynucleotides, cells, and vectors provided herein. The term “pharmaceutical composition,” as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservatives, and antioxidants can also be present in the formulation. The terms, such as “excipient,” “carrier,” “pharmaceutically acceptable carrier,” or the like, are used interchangeably herein.

In some embodiments, the pharmaceutical composition is formulated for delivery to a subject, e.g., for delivering a gene editing agent using a VLP. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In another aspect, an article of manufacture containing VLPs and/or other materials useful for the treatment of the diseases is provided. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Kits and Cells

The VLPs, libraries, nucleocapsid protein variants, polynucleotides, and vectors of the present disclosure may be assembled into kits. In some embodiments, the kit comprises polynucleotides for expression and assembly of the VLPs described herein. In other embodiments, the kit comprises VLP libraries as described herein.

The kits described herein may include one or more containers housing components for performing the methods described herein, and optionally instructions for use. Any of the kits described herein may further comprise components needed for producing or delivering VLPs as described herein. Each component of the kits, where applicable, may be provided in liquid form (e.g., in solution) or in solid form, (e.g., a dry powder). In certain cases, some of the components may be reconstitutable or otherwise processible (e.g., to an active form), for example, by the addition of a suitable solvent or other species (for example, water), which may or may not be provided with the kit.

In some embodiments, the kits may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kits may include other components depending on the specific application, as described herein.

The kits may contain any one or more of the components described herein in one or more containers. The components may be prepared sterilely, packaged in a syringe, and shipped refrigerated. Alternatively, they may be housed in a vial or other container for storage. A second container may have other components prepared sterilely. Alternatively, the kits may include the active agents premixed and shipped in a vial, tube, or other container.

The kits may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box, or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. Some aspects of this disclosure provide kits comprising a nucleic acid construct comprising a nucleotide sequence encoding the various components of the VLPs described herein (e.g., including, but not limited to, the cargo, gag proteins, gRNAs, and viral envelope glycoproteins). In some embodiments, the nucleotide sequence(s) comprises a heterologous promoter (or more than a single promoter) that drives expression of the VLP system components. In some embodiments, the kit comprises a VLP library, or a library of cells comprising a VLP library.

Other aspects of this disclosure provide kits comprising one or more nucleic acid constructs encoding the various components of the VLPs described herein. In some embodiments, the nucleotide sequence comprises a heterologous promoter that drives expression of the VLP system components.

Cells that may contain any of the VLPs, libraries, nucleocapsid protein variants, polynucleotides, and vectors described herein include prokaryotic cells and eukaryotic cells. In various aspects relating to the production of VLPs, the disclosure provides for any suitable cells for use as a VLP-producer cell line, i.e., the cell line that in various embodiments becomes transiently transformed with the plasmids encoding the protein and nucleic acid components of the VLPs. In various other aspects relating to applications of VLPs, the disclosure provides for any suitable target or recipient cells, e.g., a diseased cell or tissue in a subject in need of treatment by way of base editing or prime editing as delivered by a VLP. The methods described herein may be used to deliver a cargo such as a base editor or prime editor into a eukaryotic cell (e.g., a mammalian cell, such as a human cell). In some embodiments, the cell is in vitro (e.g., cultured cell). In some embodiments, the cell is in vivo (e.g., in a subject such as a human subject). In some embodiments, the cell is ex vivo (e.g., isolated from a subject and may be administered back to the same or a different subject).

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### EXAMPLES

### Example 1. Directed Evolution of Engineered Virus-Like Particles (eVLPs) with Desired Properties

The ability to deliver macromolecules into target cells within the body is required for many emerging therapeutic modalities, including genome editing therapies (Raguram et al., Cell 2022, 185, 2806-2827). Existing viral methods for delivering genome editing agents, including adeno-associated virus (AAV)-mediated delivery, offer potent efficiencies and programmable tissue-targeting capabilities. However, viral delivery carries increased risks of off-target editing due to prolonged editor expression, as well as increased risks of oncogenic viral genome insertions in transduced cells (Raguram et al., Cell 2022, 185, 2806-2827). Existing non-viral methods, including lipid nanoparticle (LNP)-mediated delivery, offer the benefits of transient editor expression and minimized off-target editing, but are mostly restricted to liver delivery and are generally incompatible with ribonucleoprotein (RNP) delivery of genome editing agents (Raguram et al., Cell 2022, 185, 2806-2827).

Engineered virus-like particles (eVLPs), a new platform for delivering genome editing agents as RNPs that combines key advantages of both viral and non-viral delivery, were recently developed (Banskota et al., Cell 2022, 185, 250-265). In eVLPs, the desired cargo protein is fused to a retroviral capsid protein via a flexible linker sequence, which directs packaging of the cargo into particles as they form. This linker sequence is also cleaved by the co-packaged viral protease following particle formation, enabling cargo delivery into transduced cells. By systematically engineering VLP architectures to improve cargo release, cargo loading, and component stoichiometry, fourth-generation (v4) eVLPs were developed with substantially improved potency compared to first-generation (v1) designs. These improvements in potency were essential for achieving efficient in vivo delivery across multiple organs in mice. v4 eVLPs are therefore promising new delivery vehicles that compare favorably to current state-of-the-art delivery vectors in clinical use but with the additional benefits of transient protein delivery (Banskota et al., Cell 2022, 185, 250-265).

Continued improvements to eVLP properties would enhance their therapeutic potential. Specifically, improving the production yield or potency per particle would facilitate the use of eVLPs in large animal models or clinical studies. Additionally, expanding the cell-type targeting capabilities of eVLPs would enable new strategies to treat diseases for which the delivery of therapeutics to non-liver tissues is essential (Raguram et al., Cell 2022, 185, 2806-2827). A directed evolution approach, which would involve generating large libraries of eVLP variants followed by systematically identifying specific eVLP variants with desired properties, could provide a generalizable method for developing next-generation eVLPs. Indeed, similar directed evolution approaches have been used extensively to improve the properties of various viral delivery modalities (Tabebordbar et al., Cell 2021, 184, 4919-4938; Deverman et al., Nat. Biotechnol. 2016, 34, 204-209). In these approaches, researchers have generated libraries of viral variants such that the identity of a particular variant is encoded on the viral genome packaged within that particular variant, which enables the identification of desired variants by sequencing the genomes of viruses that are present post selection. However, a major challenge with applying similar directed evolution approaches to eVLPs is the fact that eVLPs do not package any viral genetic material, which complicates the identification of desired eVLP variants post selection. Therefore, to improve the properties of eVLPs using directed evolution, new strategies are needed.

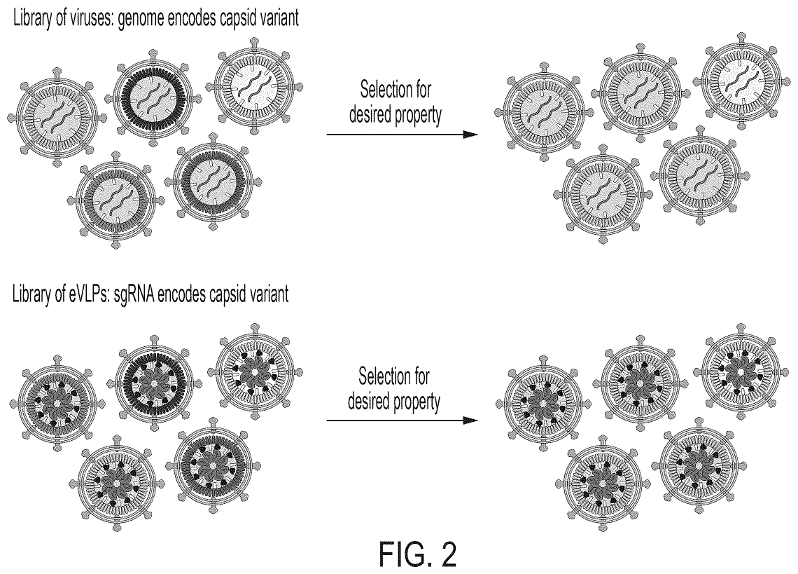

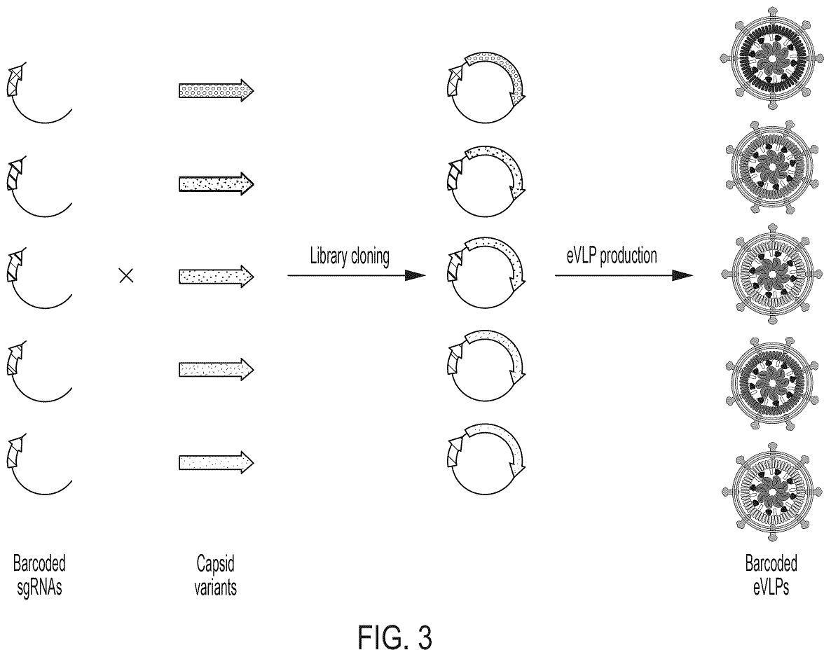

This Example describes the development of a platform for the directed evolution of engineered virus-like particles (eVLPs) with desired properties. In this platform, the identity of a particular eVLP variant is uniquely encoded by a barcode sequence present on the guide RNA molecules that are loaded within that particular eVLP variant (FIG. 3). Therefore, the identity of the eVLP variants that survive after a selection can be determined by sequencing the barcodes of the guide RNAs present after selection (FIG. 2). This scheme was applied to evolve eVLP capsid variants that exhibit improved properties compared to the previous-best v4 eVLPs. This work establishes the barcoded eVLP scheme as a promising and generalizable platform for evolving eVLP variants with desired properties. A number of next-generation eVLP variants that were identified by this platform and possess improved potency relative to the previously disclosed state-of-the-art version 4 (v4) eVLPs (e.g., as described in International Patent Application No. PCT/US2022/080834, filed Dec. 2, 2022) are also disclosed.

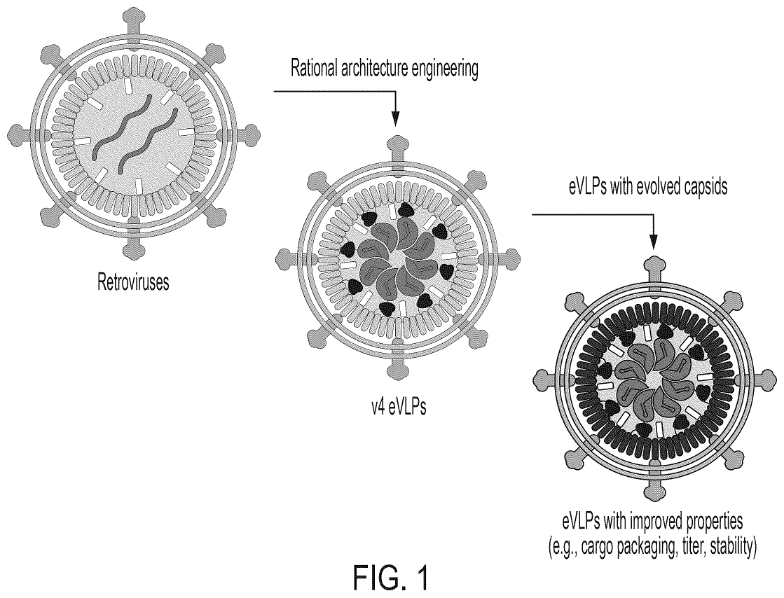

Previously, rational architecture engineering was used to convert natural retroviruses into engineered virus-like particles (eVLPs) that efficiently package and deliver therapeutic protein or RNA cargos. However, eVLPs still utilize wild-type viral capsid proteins that have been optimized by natural evolution to package viral genomes, not desired cargos. Therefore, a system that enables the directed evolution of eVLPs with desired properties would enable the eVLP capsid to be evolved to package desired cargos instead of viral genomes and also enables approaches to identify eVLPs with other improved properties (FIG. 1).

Because eVLPs lack genetic material, traditional directed evolution schemes (such as those commonly used for evolving viruses with desired properties) cannot be used. In virus-based directed evolution, each virus variant packages a genome that encodes that particular variant's identity (FIG. 2, top). After applying a selection for desired properties, the genomes of surviving viruses are sequenced, which enables the identification of viruses that possess that desired property. It was envisioned that eVLP libraries could be produced in such a way that each eVLP variant packages a guide RNA containing a barcode sequence that encodes that particular eVLP variant's identity (FIG. 2, bottom). After applying a selection for desired properties, the guide RNAs are sequenced, and the enriched barcodes are quantified, which enables the identification of eVLPs that possess that desired property.

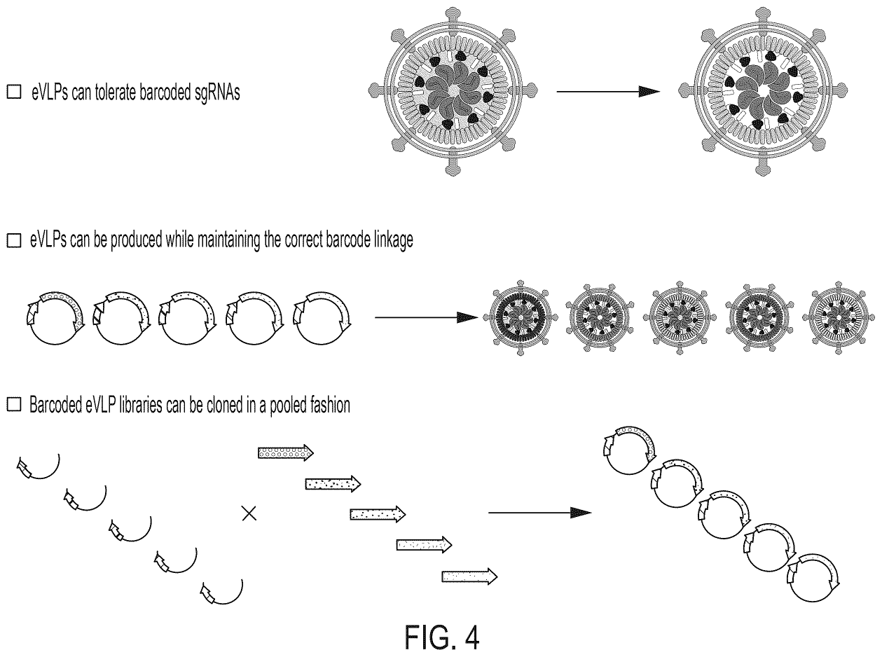

In this barcoded eVLP library approach, each capsid variant is paired with a unique barcode sequence through an optimized library cloning procedure. Then, these barcoded vectors are used to produce barcoded eVLPs in a pooled fashion, ensuring that each capsid variant only packages its cognate barcode, maintaining the specified variant:barcode linkage (FIG. 3). Key features of the barcoded eVLP evolution platform are shown in FIG. 4.

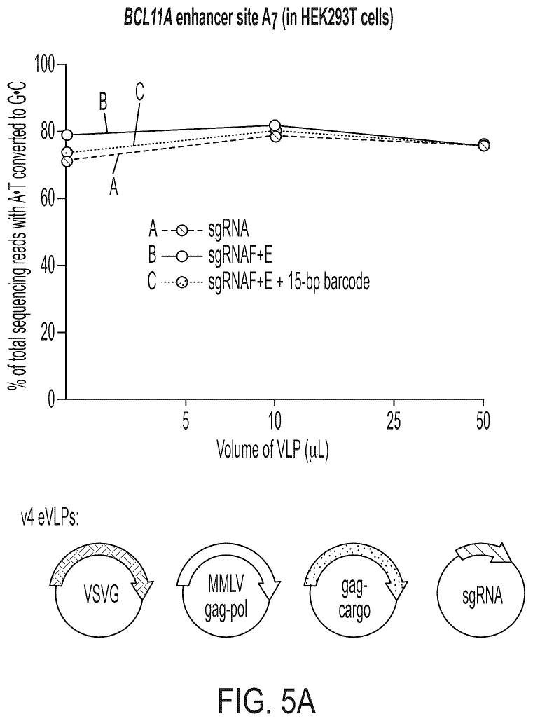

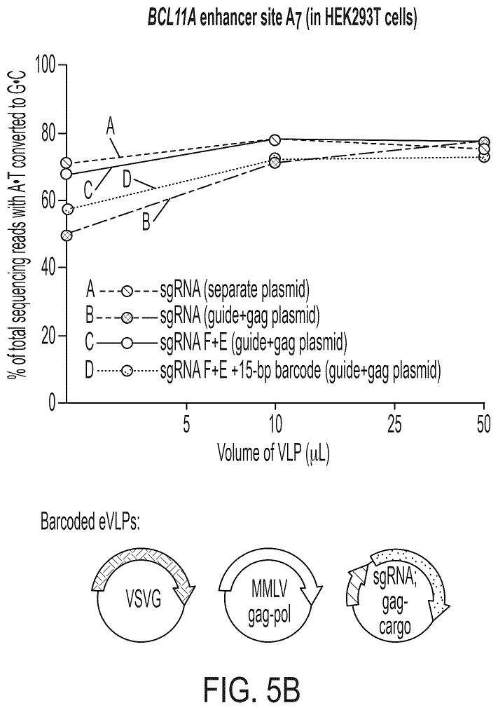

Next, the basic principles of this barcoded eVLP strategy were validated. First, it was confirmed that barcoded sgRNAs were tolerated in eVLPs. In principle, the barcode could be inserted into the sgRNA in the protospacer, as a 3′ extension, or internally within the sgRNA scaffold. The barcode was inserted internally within the sgRNA scaffold, as this enables facile PCR to retrieve the barcode sequences using primers that recognize each end of the sgRNA (which would be identical across all possible sgRNAs). Additionally, existing literature has demonstrated that short (<25-bp) barcode sequences can be inserted into the guide RNA at the P1 stem/tetraloop, without disrupting Cas9 function (Zhu, Wei et al. Genome Biol. 20, 20 (2019); Shechner, Rinn et al. Nat. Methods 12, 664-670 (2015)). Based on this precedent, a 15-bp barcode was inserted into the guide RNA, encoding for 4¹⁵~1 billion possible barcode sequences. eVLPs were produced packaging an ABE and either a canonical sgRNA, an sgRNA with the modified F+E scaffold (Chen et al., Cell 2013, 155, 1479-1491), or an sgRNA with the modified F+E scaffold along with a 15-bp tetraloop insertion. All three eVLP constructs exhibited virtually identical potency in HEK293T cells (FIGS. 5A-5B), confirming that barcoded sgRNAs were tolerated in eVLPs. Experimental validation that eVLPs can tolerate barcoded guide RNAs and can be produced using a 3-plasmid system, instead of the 4-plasmid system disclosed previously in v4 eVLPs, was also confirmed (FIGS. 5A-5B).

Next, the design of barcoded eVLP expression vectors was optimized. In barcoded eVLPs, the expression of a barcoded sgRNA must be linked to the expression of the eVLP component that varies during the directed evolution campaign. Since the original v4 eVLP production strategy employed separate expression vectors for each component of the eVLP architecture (envelope, gag-pro-pol, capsid-cargo fusion, and sgRNA) (Banskota et al., Cell 2022, 185, 250-265), it was validated that a single vector containing both an sgRNA cassette and a structural component could support efficient eVLP production. Placing the sgRNA cassette directly upstream of the capsid-cargo fusion cassette on a single vector led to efficient eVLP production at levels comparable to those achieved by the previous-standard dual-vector system (FIGS. 5A-5B). Collectively, these initial validation experiments suggested that eVLPs packaging barcoded sgRNAs could be efficiently produced.

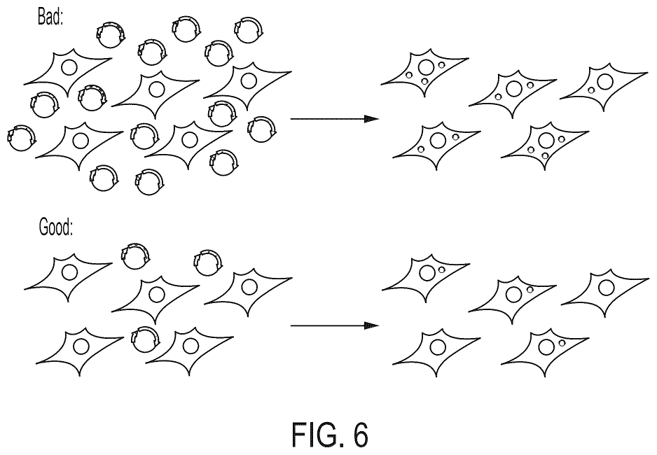

A key requirement of this platform is the fact that the barcode linkage must be maintained during eVLP production. This requires only one library member per producer cell (FIG. 6). If more than one library member is present in each producer cell, then each producer cell would produce a mix of eVLP variants that package a mix of barcodes instead of their single, cognate barcode. The illustration in FIG. 6 shows that eVLPs produced in this scenario would package non-cognate barcodes (each barcode on the guide RNA should be the same color as the capsid in a perfectly barcoded situation). With only one library member per producer cell, each cell produces a single eVLP variant packaging a single cognate barcode sequence, thereby maintaining barcode linkage.

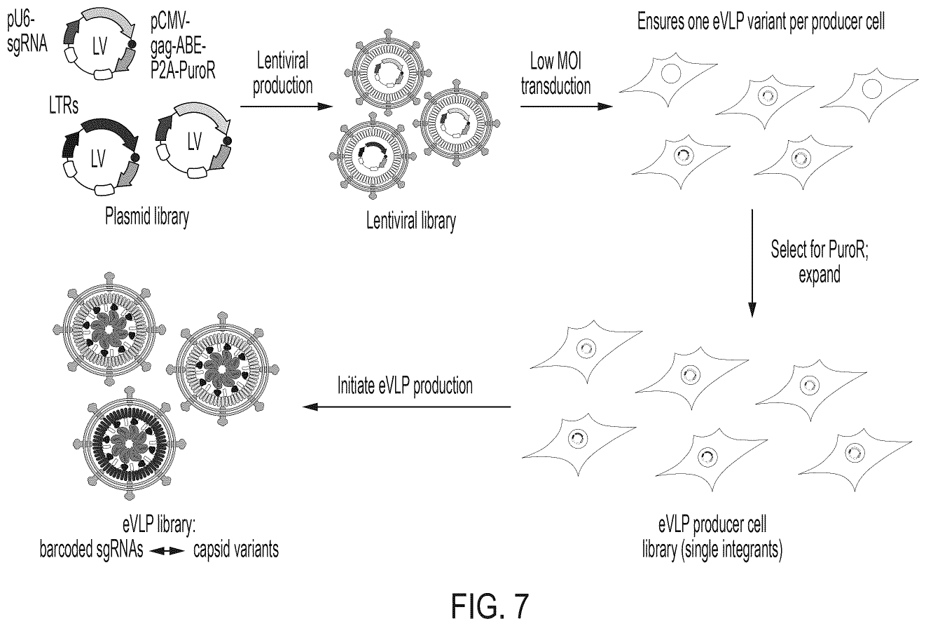

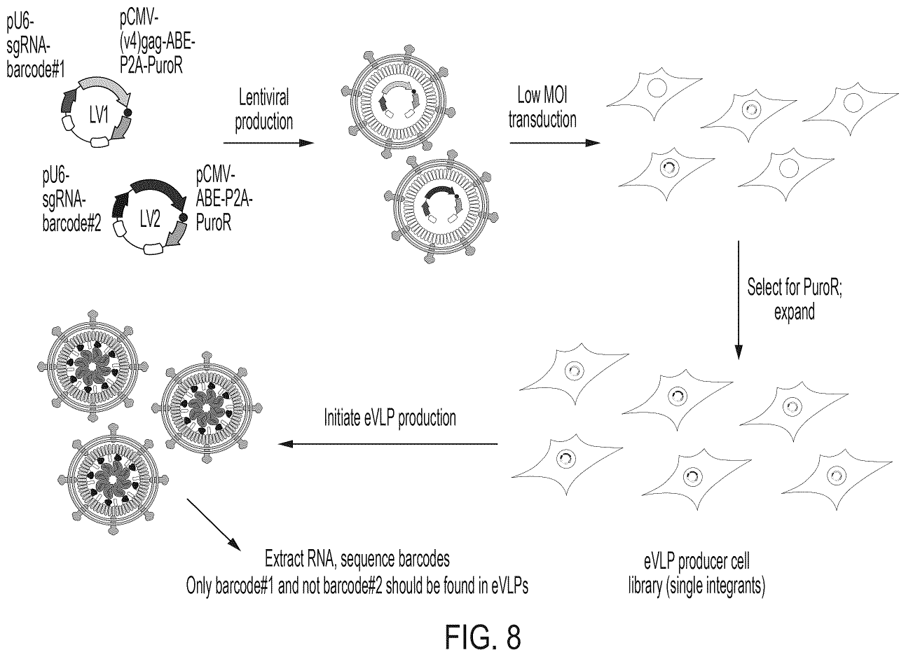

Low-MOI lentiviral transduction is a common method for ensuring a single vector per cell in pooled screens (Piccioni et al., Curr. Protoc. Mol. Biol. 2018, 121). A strategy for producing barcoded eVLP libraries was developed that first involved generating a barcoded producer cell library using lentiviral transduction, followed by initiating eVLP production from the singly integrated producer cell library (FIG. 7). FIG. 7 illustrates a scheme for generating barcoded eVLP libraries that maintain barcode linkage. First, a barcoded plasmid library is generated (using cloning methods described in FIG. 8). Next, this barcoded plasmid library is used to produce a lentiviral library. Then, this lentiviral library is used to generate a producer cell library in which each producer cell contains a single integration of a cassette expressing an eVLP capsid variant and its cognate barcode. Finally, this producer cell library is used to generate a pooled library of barcoded eVLPs.

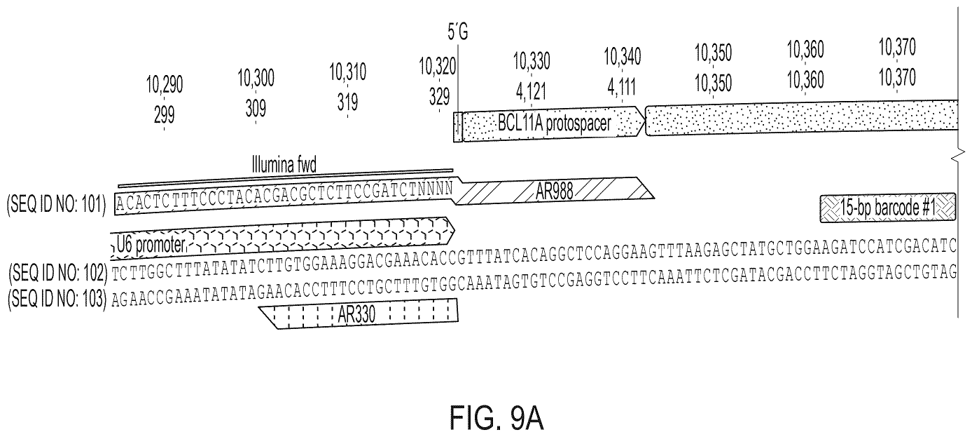

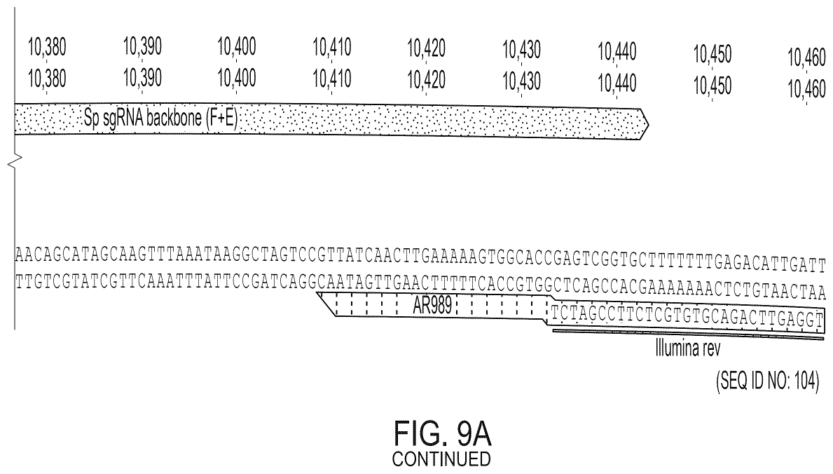

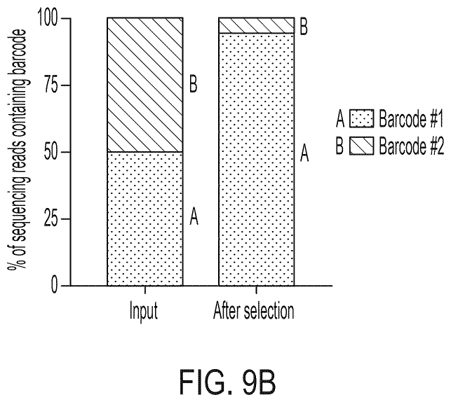

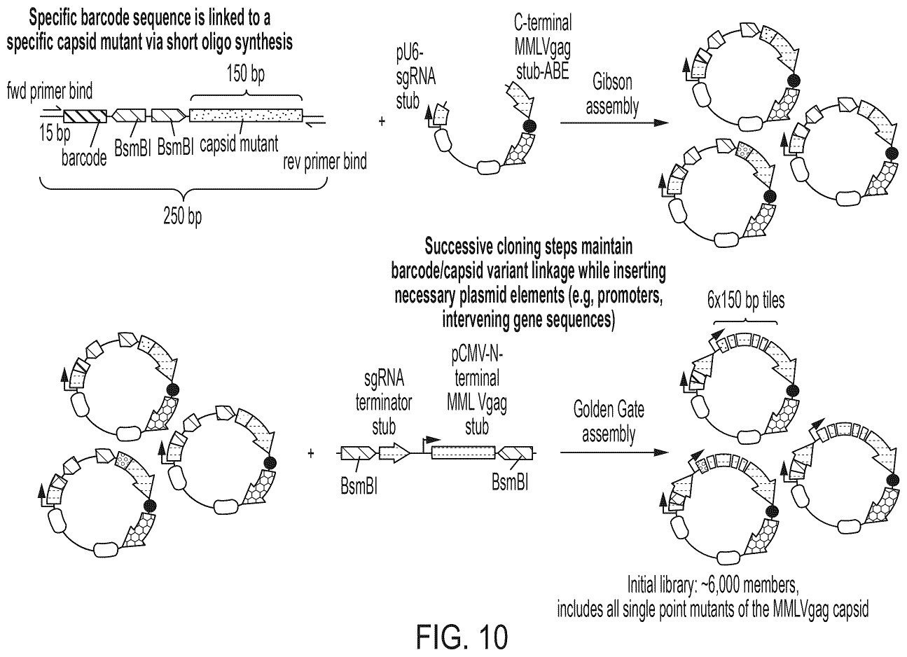

To validate that this scheme maintains barcode linkage, a simplified version of the scheme using only two barcodes and two capsid variants was performed. The first barcode was paired with the functional v4 eVLP capsid. The second barcode was paired with a non-functional eVLP capsid. Therefore, if barcode linkage is successfully maintained, then only the first barcode and not the second barcode should be found in eVLPs (FIG. 8). Following the generation of a mixture of producer cells in which each cell expressed one of these two constructs, eVLP production was initiated, and the sgRNAs packaged into the resulting eVLPs were sequenced. Indeed, upon sequencing guide RNAs packaged in eVLPs, it was observed that a 50:50 input mixture resulted in a 95:5 ratio of observed first barcode to second barcode in eVLPs, indicating that barcode linkage was successfully maintained (FIGS. 9A-9B). This result suggested that the lentiviral scheme does faithfully maintain barcode linkage by ensuring only one barcoded eVLP variant per producer cell and indicated that the scheme in FIG. 8 could be used to successfully generate barcoded eVLP libraries.

With a validated workflow for producing barcoded eVLPs in hand, libraries of eVLP variants that could be screened to identify variants that possess desired properties were designed and constructed. Initial efforts were focused on generating a barcoded library of eVLP capsid variants. In v4 eVLPs, the retroviral capsid proteins that are used are identical to the capsids used by wild-type retroviruses. These capsids have evolved in nature to be optimal for packaging viral genomes (Basyuk et al., J. Mol. Biol. 2005, 354, 330-339) but are likely not optimal for packaging large, non-native protein cargos in eVLPs. Remodeling the internal eVLP capsid surfaces to optimize for protein cargo packaging instead of viral genome packaging could therefore substantially improve eVLP properties, including potency per particle, overall particle yield or titer, and particle stability.

To begin to evolve eVLP capsids using the barcoded eVLP platform, an initial barcoded eVLP library was designed, containing ~4,000 single-residue mutants of the capsid and nucleocapsid domains of the retroviral gag used in the v4 eVLP architecture. A two-step cloning strategy was designed to enable library cloning in a pooled fashion while maintaining the specified linkage between a particular barcode sequence and a particular capsid mutant (FIG. 10). This library construction strategy ensures complete knowledge of barcode/mutant linkage a priori that can be used to decode the results of selections after barcode sequences alone are retrieved from eVLPs. The gene region for mutagenesis was divided into smaller chunks such that each chunk was short enough to be synthesized using commercially available high-throughput oligo synthesis methods. In this way, the entire library was divided into four sub-libraries of ~1,000 members each, and each sub-library oligo pool was synthesized separately. Each synthesized oligo within each pool contained a specified barcode/mutant pair, internal BsmBI recognition motifs, and common primer handles on either end to allow for oligo amplification during the cloning process. Barcode sequences were chosen such that they avoided BsmBI recognition motifs, homopolymers >2 bp, and excessively high or low GC content to ensure well-behaved sequences. Barcode sequences were also chosen such that no two barcode sequences were within four mismatches of each other to minimize the likelihood of incorrect barcode classification due to sequencing errors during the final barcode retrieval steps. Amplified oligo pools were ligated into a lentiviral vector backbone via Gibson assembly (FIG. 10). Then, in a second cloning step, each sub-library pool was subjected to Golden Gate assembly to drop in the intervening sequences: sgRNA terminator, CMV promoter, and N-terminal portion of gag (FIG. 10). This two-step cloning process enables complete control of barcode/mutant linkage by synthesizing them as user-defined short oligo pools and maintains the specified barcode linkage even while inserting multiple kilobases of additional DNA in between the barcode/mutant pieces.

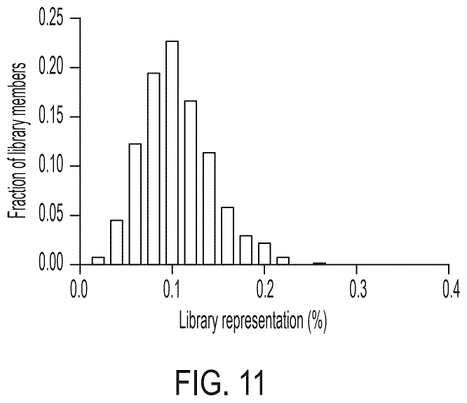

Overall, FIG. 10 details the optimized cloning scheme for generating a barcoded capsid library. First, a specified barcode sequence is linked to a specified capsid variant on a short (<300 bp) oligonucleotide via commercially available, high-throughput pooled oligo synthesis. In this way, the correspondence between barcode and capsid variant is defined from the beginning and maintained throughout the cloning process. This pool of barcoded oligonucleotides is amplified using PCR under highly optimized conditions (low cycle number, low template concentration) to minimize recombination and loss of barcode linkage. The PCR-amplified products are subsequently ligated into a digested backbone vector using Gibson assembly. In a second step, the pooled library is subjected to Golden Gate assembly using a donor sequence that replaces the intervening sequence between the barcode and capsid variant in order to provide essential sequences (e.g., guide RNA terminator, promoter to drive capsid expression, and N-terminal fragment of the capsid). To validate successful library construction, the barcodes present in the final assembled plasmid pool of one sub-library (~1,000 members) were sequenced. Libraries cloned using this procedure are high quality and possess good coverage of all barcode sequences with a median representation frequency of 0.1% (FIG. 11).

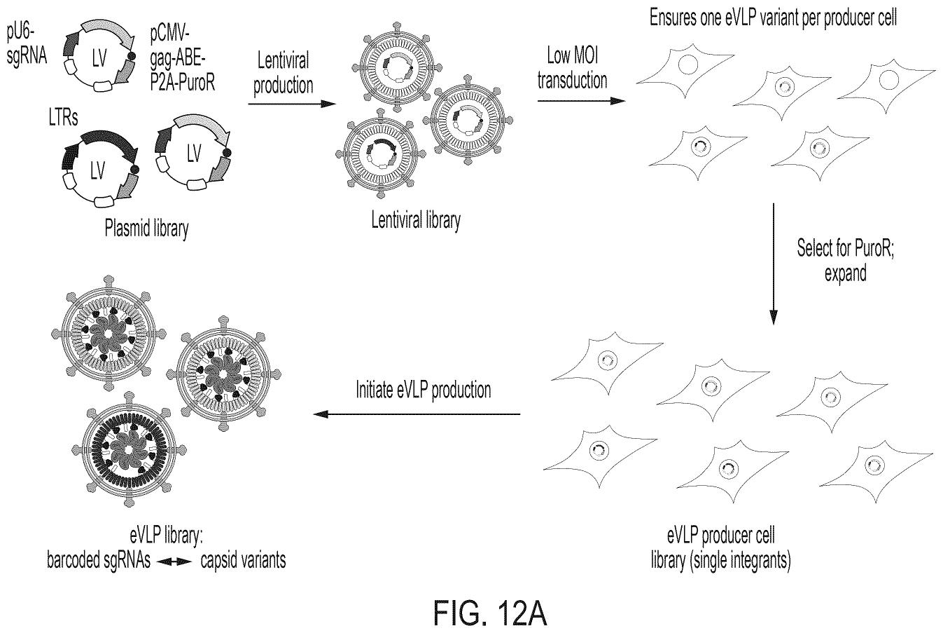

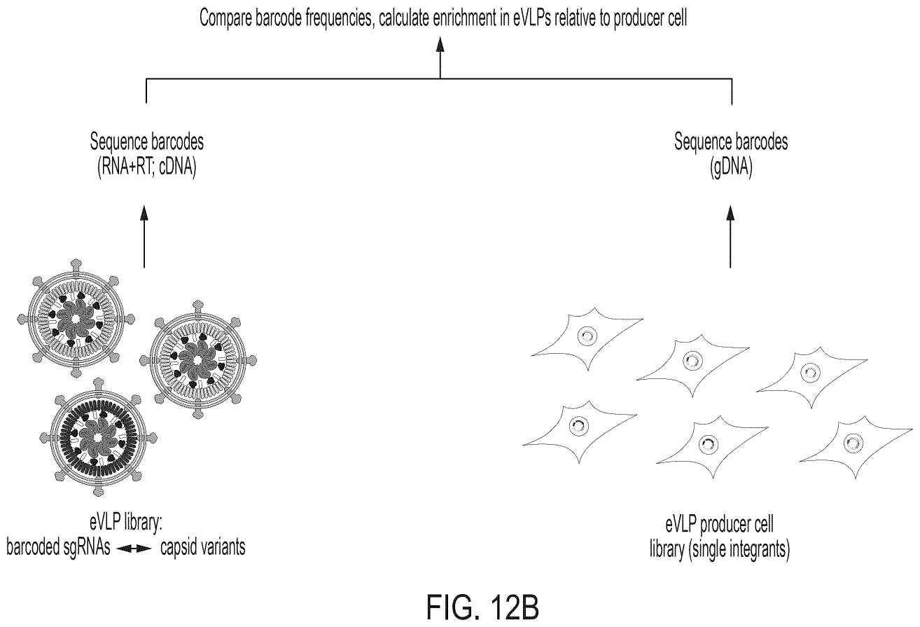

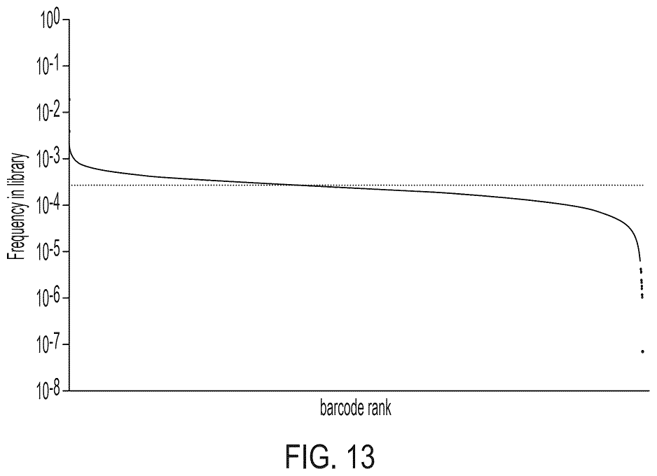

For initial screens, the built-in selection that is present when producing eVLPs from the producer cell library was used, as detailed in FIG. 12A. Barcode sequences that are enriched in eVLPs relative to producer cells correspond to eVLP variants that either package more cargo per particle or are produced at higher titer. Barcode sequences that are de-enriched in eVLPs relative to producer cells correspond to eVLP variants that are less functional and therefore undesired (FIG. 12B). Barcode coverage in producer cells was assessed using high-throughput sequencing (FIG. 13). First, the sgRNA barcodes present in the genomic DNA of the producer cell library generated by lentiviral transduction and puromycin selection were sequenced. This analysis revealed that ~99% of all barcode sequences were retrieved from producer cells, suggesting sufficient coverage of the library (FIG. 13). Next, the sgRNA barcodes present inside eVLPs that were generated from this producer cell library were sequenced, and the fold change in barcode frequency present in the eVLP RNA fraction compared to the producer cell gDNA fraction was calculated. Barcodes enriched in eVLPs relative to producer cells are expected to correspond to capsid variants that support higher-titer eVLP production or greater cargo packaging efficiency.

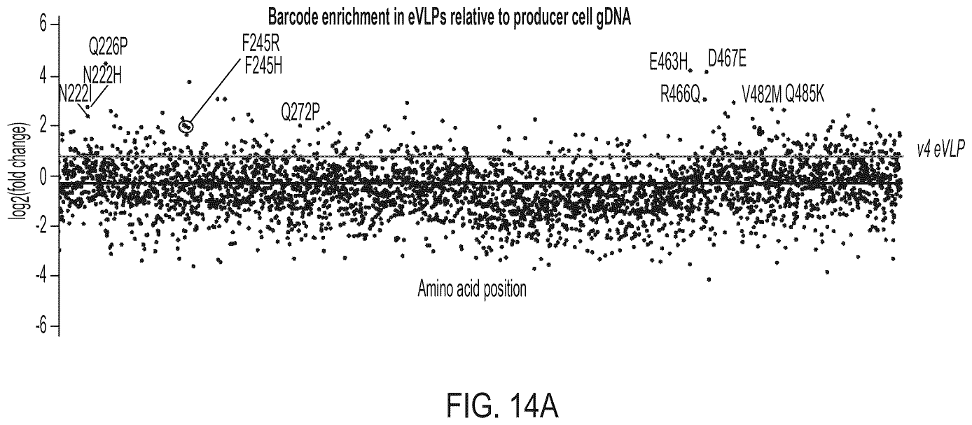

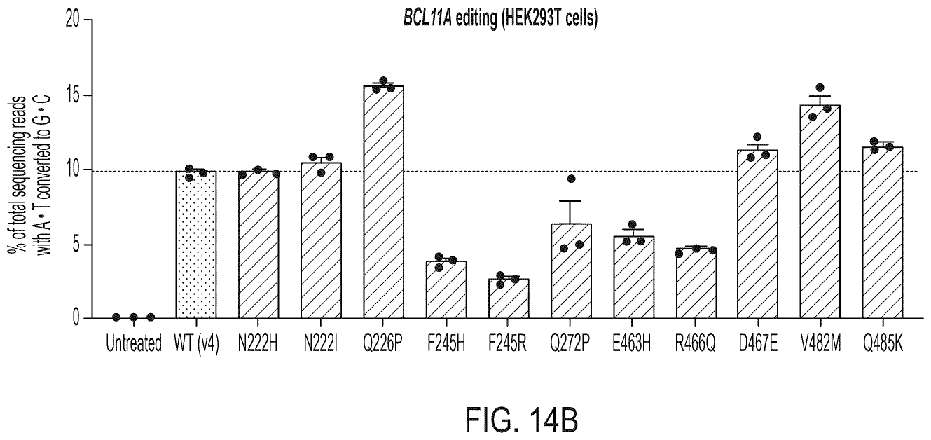

Quantification of barcode enrichment in eVLPs relative to producer cells was performed next. The value obtained for the v4 eVLP capsid is shown as a line labeled “v4 eVLP” (FIG. 14A). Barcodes enriched at frequencies greater than 2-fold (i.e., plotted above the line labeled “v4 eVLP”) correspond to variants that in principle possess improved titer or cargo packaging compared to v4 eVLPs and are therefore promising candidates for next-generation eVLPs. A complete list of all promising variants identified by this barcoded eVLP screen is provided in Table 1. A set of eVLP mutants corresponding to the enriched barcodes above were tested individually for their ability to transduce and edit the BCL11A enhancer locus in HEK293T cells. Importantly, several eVLP mutants (e.g., Q226P, D467E, V482M, and Q485K) exhibited improved potency relative to the previous best v4 eVLP (FIG. 14B). Additional beneficial mutants are found by applying a selection for target cell transduction instead of production only.

The barcode corresponding to the wild-type v4 eVLP capsid was enriched by approximately 2-fold, and the majority of capsid single mutants were enriched by less than 2-fold (FIG. 15A). This result suggested that the majority of mutants perform worse in eVLPs compared to the wild-type capsid, as most mutations might destabilize the eVLP capsid. Encouragingly, several barcodes were enriched by >2-fold, suggesting that their corresponding variants might support higher-titer eVLP production or greater cargo packaging efficiency compared to v4 eVLPs (FIG. 15A).

In addition to investigating the effect of capsid mutants on eVLP production, the effect of capsid mutants on eVLP transduction was also investigated. HEK293T cells were transduced with purified barcoded eVLPs, successfully delivered sgRNAs were retrieved from the transduced cells six hours post infection, the barcodes retrieved from the transduced cells were sequenced, and the fold change in barcode frequency in the retrieved barcodes compared to the initial purified eVLPs was calculated. The vast majority of barcodes enriched worse compared to the wild-type capsid (FIG. 15B). In fact, the results of both production and transduction screens revealed that almost all of the capsid mutants that supported improved production exhibited decreased transduction efficiencies relative to the wild-type capsid, although the magnitude of this decrease was not large in most cases (FIG. 15C).

To further investigate the properties of the eVLP capsid variants that enriched in the screens, a subset of enriched hits were cloned and tested individually to assess their delivery potency into HEK293T cells. A few of these variants exhibited improved potency relative to v4 eVLPs by up to 3-fold (FIG. 15D). Potency-improving mutants seemed to cluster at the N- and C-termini of the capsid protein, which represent two distinct subdomains of the capsid, raising the possibility that they might synergize to yield additive improvements in eVLP potency. These results indicate that barcoded eVLP production and transduction screens can successfully nominate eVLP capsid variants that exhibit improved potency compared to the previous state-of-the-art v4 eVLPs.

Overall, barcoded eVLP libraries provide a platform for identifying eVLPs with desired properties. Selections that include transduction of high-value cell types can be applied to identify eVLP variants that can successfully transduce those cell types (FIG. 15). The mutants listed in Table 1, which were discovered by the barcoded eVLP evolution platform described herein, represent next-generation eVLP variants with improved titer and/or cargo packaging abilities.

In principle, capsid mutants identified from the eVLP evolution platform described herein can be incorporated into either the gag-cargo (e.g., ABE) fusion protein, the gag-pro-polyprotein, or both (FIG. 16). A selection of the identified C-terminal capsid mutants in the gag-cargo fusion were paired with gag-pro-polyproteins containing the same C-terminal mutations, and several combinations were observed to exhibit improved potency (FIG. 17A). The R501I mutant displayed particularly improved potency, and combination with the Q226P mutant in the gag-cargo fusion protein showed additional improvements. Addition of the Q226P mutation into the gag-pro-polyproteins had different effects depending on whether Q226P was also present in the gag-cargo fusion (FIG. 17B). In general, addition of Q226P in the gag-pro-polyprotein positively impacts efficiency when paired with a Q226P mutation in the gag-cargo fusion (FIG. 17B, right side). The best improvements in potency were observed when only the Q226P mutation was incorporated into the gag-pro-polyprotein in combination with a C-terminal mutant (in particular, R501D) in the gag-cargo fusion protein (FIG. 17C). This suggests that the Q226P and C-terminal mutants influence eVLP potency by different mechanisms (e.g., different rates of capsid subunit cleavage in the gag-cargo fusion vs. gag-pro-pol, or differences in stability requirements of the gag-cargo fusion vs. gag-pro-pol).

The barcoded eVLP platform described herein provides a powerful strategy for developing next-generation eVLPs with desired properties. The development and successful implementation of optimized methods for designing, constructing, producing, and screening barcoded eVLP libraries reported herein will facilitate the continued improvement of the therapeutic applicability of eVLP technology.

Thus far, the application of barcoded eVLP screening to identify capsid variants that exhibit improved potency compared to previous-best v4 eVLPs has been demonstrated. These initial screens have illuminated the fitness landscape of single-substitution capsid mutants and their influence on both eVLP production and eVLP transduction. Assessing combinations of the identified single mutants revealed that mutations can synergize to further improve eVLP potency.

Beyond identifying capsid mutants that exhibit improved potency, this barcoded eVLP evolution platform can be used to evolve eVLPs with other desired properties. For example, eVLP capsids can be evolved to optimize the packaging of specific, high-value cargo proteins, including prime editors and other genome editing agents. Additionally, barcoded eVLPs can be used to evolve eVLP envelope proteins or other surface-exposed targeting moieties to bias eVLP transduction toward specific cell types over others.

TABLE 1
List of Next-Generation eVLP variants

| Mutation in gag protein of | Enrichment Value |
|---|---|
| SEQ ID NO: 100 | (correlates with potency) |
| Q226P | 4.533316327 |
| E463H | 4.258586212 |
| D467E | 4.206369733 |
| S246L | 3.832545315 |
| A254W | 3.17886706 |
| T253F | 3.178392658 |
| R466Q | 3.16392567 |
| E473N | 3.045722822 |
| D297N | 3.040151826 |
| N222H | 2.880386373 |
| V482M | 2.796592648 |
| Y506L | 2.76669783 |
| Q485K | 2.766526971 |
| Y227S | 2.724486615 |
| N240A | 2.665040308 |
| H471M | 2.642925704 |
| L260M | 2.597321045 |
| D500Q | 2.569855773 |
| P229D | 2.555322023 |
| N222I | 2.54032276 |
| R472E | 2.518807042 |
| D297A | 2.482222104 |
| S244Y | 2.449471544 |
| L477W | 2.443532447 |
| I256V | 2.419273735 |
| A427G | 2.41324953 |
| I261K | 2.35566142 |
| P305R | 2.353551131 |
| S497C | 2.330852904 |
| S483W | 2.313185013 |
| A505M | 2.278781084 |
| T279G | 2.266403917 |
| F245H | 2.21120559 |
| Q272P | 2.174297661 |
| F245R | 2.157350549 |
| K292L | 2.15077873 |
| W228N | 2.145952121 |
| L499Y | 2.134999313 |
| C507F | 2.13472049 |
| K418Y | 2.126246253 |
| L478K | 2.120102147 |
| L235C | 2.119463843 |
| R496L | 2.118485608 |
| K239A | 2.113254872 |
| R470A | 2.106827052 |
| L277W | 2.104132476 |
| V308R | 2.095989528 |
| S246H | 2.095149617 |
| F230C | 2.082237362 |
| A479Q | 2.077935516 |
| A293H | 2.034205106 |
| T276R | 2.026986282 |
| A479L | 2.012362261 |
| L499F | 2.011457752 |
| R495Y | 1.992886575 |
| K239T | 1.962274694 |
| A219C | 1.957926627 |
| E289C | 1.956289671 |
| R468Q | 1.944946585 |
| R218I | 1.93499506 |
| N240E | 1.933123366 |
| T480H | 1.926186261 |
| D234E | 1.912123736 |
| D298Y | 1.901111434 |
| S231Y | 1.894759942 |
| H512Q | 1.891780487 |
| E281T | 1.890440857 |
| A479K | 1.876501295 |
| P216K | 1.850728399 |
| A479S | 1.84866314 |
| C507V | 1.848487817 |
| G491C | 1.846155849 |
| L499G | 1.845797858 |
| Q485G | 1.83864374 |
| H471D | 1.835393322 |
| E438H | 1.834232819 |
| F215L | 1.831420913 |
| T436N | 1.825033238 |
| F245W | 1.824532592 |
| L288D | 1.824383011 |
| Q264S | 1.819315492 |
| K510M | 1.817691719 |
| G492Q | 1.806108574 |
| H512S | 1.791466251 |
| E452S | 1.788156056 |
| R501D | 1.784159215 |
| K508D | 1.783763777 |
| E458F | 1.779721363 |
| L288K | 1.767928293 |
| S483L | 1.752760749 |
| D500G | 1.746133559 |
| N237D | 1.738513492 |
| S483R | 1.7047682 |
| V424Y | 1.704495126 |
| Y236E | 1.69382589 |
| G223K | 1.675346771 |
| R495K | 1.675161281 |
| G421D | 1.666681039 |
| E441M | 1.666598767 |
| G492W | 1.664160025 |
| H471G | 1.662721942 |
| K486L | 1.662421177 |
| R446G | 1.661746377 |
| D502Q | 1.661166598 |
| G221W | 1.655990621 |
| R501I | 1.649261382 |
| I261W | 1.64655049 |
| E460W | 1.645777997 |
| V481T | 1.645518559 |
| P216I | 1.632008465 |
| S233K | 1.631592863 |
| E307N | 1.628079885 |
| V482L | 1.618601847 |
| Q272G | 1.617054121 |
| E509G | 1.606399618 |
| S258W | 1.602434457 |
| T266C | 1.59523586 |
| G511L | 1.595084515 |
| S231F | 1.594621917 |
| E449D | 1.575626817 |
| E281N | 1.570156052 |
| S233I | 1.549874727 |
| I430W | 1.545353264 |
| V259R | 1.544207503 |
| D488Q | 1.541092555 |
| T262N | 1.536001725 |
| C507G | 1.533130182 |
| W238D | 1.531576128 |
| Q487P | 1.529512529 |
| I444E | 1.522458558 |
| G280W | 1.521408095 |
| T276V | 1.519735185 |
| L255H | 1.496971073 |
| N240L | 1.495843945 |
| M474W | 1.493396505 |
| E509A | 1.492908411 |
| A505Y | 1.491008483 |
| R489A | 1.488667381 |
| V286H | 1.486937068 |
| E447H | 1.484016071 |
| G250C | 1.483019425 |
| C504N | 1.48121804 |
| G250D | 1.48089067 |
| R496N | 1.477834756 |
| D488I | 1.475120084 |
| G492H | 1.470538835 |
| G250L | 1.469064347 |
| Q490T | 1.468627161 |
| G511K | 1.46781923 |
| G296D | 1.461556001 |
| T448M | 1.461019563 |
| L274W | 1.457666683 |
| F215G | 1.456257782 |
| D502A | 1.442784078 |
| Q498K | 1.441560352 |
| Y236M | 1.439790766 |
| T262W | 1.435720479 |
| R446I | 1.421679355 |
| Q485L | 1.418756884 |
| W228F | 1.415971845 |
| G280D | 1.410808328 |
| N222M | 1.40720453 |
| H263I | 1.402992651 |
| D297M | 1.40164593 |
| S497P | 1.398516809 |
| R495F | 1.398184822 |
| R489K | 1.396967994 |
| Q224L | 1.382134248 |
| E257A | 1.381840994 |
| L304N | 1.378827467 |
| I261Q | 1.378357783 |
| S497Q | 1.377667043 |
| Q271F | 1.375898934 |
| R496F | 1.367623428 |
| P301S | 1.357964399 |
| G491Q | 1.354735904 |
| R466A | 1.353503028 |
| S246F | 1.352441209 |
| G299M | 1.350674602 |
| H471N | 1.349182384 |
| P249S | 1.337118245 |
| Q303S | 1.336384091 |
| P305G | 1.334127491 |
| S497T | 1.332367177 |
| R469Q | 1.328253517 |
| N242T | 1.3261498 |
| L225Q | 1.325465121 |
| S233R | 1.322701887 |
| A505W | 1.321796104 |
| A311F | 1.313433255 |
| A310R | 1.31261973 |
| R495I | 1.306315492 |
| R285V | 1.304961307 |
| T480W | 1.304557516 |
| R218G | 1.302974956 |
| K283F | 1.301078151 |
| G223A | 1.297649632 |
| R295M | 1.297484909 |
| P229L | 1.295263964 |
| K433P | 1.287420861 |
| A219K | 1.287205862 |
| R489M | 1.285541706 |
| G223C | 1.281294708 |
| N241K | 1.280579836 |
| V308I | 1.279986637 |
| G299R | 1.277832762 |
| P437K | 1.269525817 |
| N222Y | 1.269351515 |
| R218H | 1.268016053 |
| G280Y | 1.264367597 |
| K510P | 1.263245468 |
| R470M | 1.262160253 |
| A219N | 1.261850673 |
| N240S | 1.260347362 |
| D500M | 1.254887788 |
| S258V | 1.245793053 |
| A311E | 1.24354875 |
| K510R | 1.242736055 |
| K486I | 1.239860192 |
| A219G | 1.239825181 |
| S244M | 1.232120798 |
| P265G | 1.231440082 |
| E435A | 1.231166921 |
| Q503T | 1.228900134 |
| L420C | 1.227945248 |
| G299Y | 1.227062785 |
| Q498V | 1.223219882 |
| R468W | 1.218752783 |
| A293Y | 1.216875795 |
| E447S | 1.213439221 |
| N432Y | 1.211987923 |
| A479Y | 1.211574433 |
| R291K | 1.207653908 |
| Q487E | 1.207154278 |
| C507W | 1.204606111 |
| S497N | 1.204280963 |
| L235M | 1.20362646 |
| K508N | 1.202107909 |
| V294Q | 1.200140069 |
| D269G | 1.196340302 |
| D297P | 1.194672259 |
| S244T | 1.194487598 |
| S246V | 1.191327633 |
| R496A | 1.190246723 |
| S232L | 1.190028598 |
| G421A | 1.189718851 |
| E281C | 1.188401307 |
| C504S | 1.187305035 |
| G491H | 1.186479619 |
| A505I | 1.186316092 |
| R440C | 1.183001141 |
| Q271A | 1.181619447 |
| Q264T | 1.180238712 |
| E282S | 1.179337808 |
| L477C | 1.176846427 |
| K486V | 1.176271323 |
| Q271D | 1.175359899 |
| G223D | 1.172064687 |
| F230E | 1.170798066 |
| M474C | 1.170547978 |
| R468I | 1.170208259 |
| M474I | 1.165640521 |
| K486E | 1.163471566 |
| E257C | 1.161311047 |
| L478V | 1.154220722 |
| P249F | 1.153572526 |
| I430H | 1.15289081 |
| A311M | 1.147192037 |
| Y506M | 1.144231801 |
| G220C | 1.144046088 |
| T480E | 1.143710867 |
| Q224R | 1.141790054 |
| E509M | 1.141496537 |
| R470W | 1.133572146 |
| T276M | 1.130602763 |
| Q224F | 1.129772925 |
| R468S | 1.129665144 |
| G421R | 1.127849927 |
| R496K | 1.124389942 |
| D234A | 1.123090051 |
| S246Y | 1.122479549 |
| S475G | 1.119671598 |
| T302V | 1.114556782 |
| K464L | 1.113215494 |
| L478P | 1.110165096 |
| R472D | 1.10470966 |
| H512M | 1.104078276 |
| D500I | 1.103966136 |
| S232A | 1.102592621 |
| P301L | 1.101625723 |
| R501L | 1.100275081 |
| T448W | 1.099926501 |
| G250R | 1.095155705 |
| G511P | 1.094637943 |
| T262F | 1.093242771 |
| K486F | 1.092016456 |
| G275W | 1.091449267 |
| E282H | 1.084976515 |
| E282Y | 1.084800912 |
| N240I | 1.083724702 |
| L477E | 1.082666303 |
| N241V | 1.07922944 |
| G221M | 1.078415744 |
| R489F | 1.075029712 |
| K239H | 1.073663537 |
| D502P | 1.069162355 |
| P249K | 1.066810995 |
| R455Q | 1.066141272 |
| Q224I | 1.064864765 |
| L499T | 1.060562312 |
| D297W | 1.059081956 |
| P305M | 1.058913638 |
| F215M | 1.057926427 |
| T262Q | 1.056264883 |
| T448V | 1.055011742 |
| N222V | 1.053850748 |
| R300L | 1.053234364 |
| Q485W | 1.047943243 |
| L260W | 1.04740112 |
| P265F | 1.0461029 |
| WT (v4 eVLP) | 1.045918 |

### Methods

### Cell Culture Conditions.

HEK293T cells (ATCC; CRL-3216) and Gesicle Producer 293T cells (Takara; 632617) were maintained in DMEM+GlutaMAX (Life Technologies) supplemented with 10% (v/v) fetal bovine serum. Cells were cultured at 37° C. with 5% carbon dioxide and were confirmed to be negative for mycoplasma by testing with MycoAlert (Lonza Biologics).

General cloning. In general, plasmids used herein were cloned using USER cloning or KLD cloning as described previously²⁵². DNA was PCR-amplified using Phusion® U Green Multiplex PCR Master Mix (Thermo Fisher Scientific). Mach1 (Thermo Fisher Scientific) or NEB® Stable (New England Biolabs) chemically competent E. coli were used for plasmid propagation.

General eVLP Production and Purification.

eVLPs were produced as described previously²⁸⁹. Briefly, eVLPs were produced by transient transfection of Gesicle Producer 293T cells. For medium- to large-scale preparations, Gesicle cells were seeded in T-75 flasks (Corning) at a density of 5×10⁶ cells per flask. After 20-24 hours, cells were transfected using the jetPRIME® transfection reagent (Polyplus) according to the manufacturer's protocols. A mixture of plasmids expressing VSV-G (400 ng), MMLVgag-pro-pol (3,375 ng), MMLVgag-3×NES-ABE8e (1,125 ng), and an sgRNA (4,400 ng) were co-transfected per T-75 flask.

Forty to forty-eight hours post-transfection, producer cell supernatant was harvested and centrifuged for 5 minutes at 500 g to remove cell debris. The clarified eVLP-containing supernatant was filtered through a 0.45-μm PVDF filter. For eVLPs that were used in cell culture, unless otherwise stated, the filtered supernatant was concentrated 100-fold using PEG-it Virus Precipitation Solution (System Biosciences; LV825A-1) according to the manufacturer's protocols and resuspended in Opti-MEM serum-free media.

General eVLP Transduction and Genomic DNA Isolation.

Cells were plated for transduction in 48-well plates (Corning) at a density of 30,000-40,000 cells per well. After 20-24 hours, BE-eVLPs were added directly to the culture media in each well. Forty-eight to seventy-two hours post-transduction, cellular genomic DNA was isolated as previously reported²⁵². Briefly, cells were washed once with PBS and lysed in 150 μL of lysis buffer (10 mM Tris-HCl pH 8.0, 0.05% SDS, 25 μg mL⁻¹ Proteinase K (Thermo Fisher Scientific)) at 37° C. for 1 hour followed by heat inactivation at 80° C. for 30 minutes.

### High-Throughput Sequencing of Genomic DNA.

Genomic DNA was isolated as described above. Following genomic DNA isolation, 1 μL of the isolated DNA (1-10 ng) was used as input for the first of two PCR reactions. Genomic loci were amplified in PCR1 using Phusion® U polymerase (Thermo Fisher Scientific). PCR1 was performed as follows: 95° C. for 3 minutes; 30-35 cycles of 95° C. for 15 seconds, 61° C. for 20 seconds, and 72° C. for 30 seconds; 72° C. for 1 minute. PCR1 products were confirmed on a 1% agarose gel. 1 μL of PCR1 was used as an input for PCR2 to install Illumina barcodes. PCR2 was conducted for nine cycles of amplification using a Phusion® HS II kit (Life Technologies). Following PCR2, samples were pooled and gel purified in a 1% agarose gel using a Qiaquick Gel Extraction Kit (Qiagen). Library concentration was quantified using the Qubit High-Sensitivity Assay Kit (Thermo Fisher Scientific). Samples were sequenced on an Illumina MiSeq® instrument (paired-end read, read 1: 200-280 cycles, read 2: 0 cycles) using an Illumina MiSeq® 300 v2 Kit (Illumina).

### High-Throughput Sequencing Data Analysis.

Sequencing reads were demultiplexed using the MiSeq® Reporter software (Illumina) and were analyzed using CRISPResso2²⁴⁴ as previously described²⁵². Batch analysis mode (one batch for each unique amplicon and sgRNA combination analyzed) was used in all cases. Reads were filtered by minimum average quality score (Q>30) prior to analysis. The following quantification window parameters were used: -w 20 -wc -10. Base editing efficiencies are reported as the percentage of sequencing reads containing a given base conversion at a specific position. Prism 9 (GraphPad) was used to generate dot plots and bar plots.

### General Lentiviral Vector Production.

HEK293T/17 (ATCC CRL-11268) cells were maintained in antibiotic-free DMEM supplemented with 10% fetal bovine serum (v/v). On day 1, 5×10⁶ cells were plated in 10 mL of media in T75 flasks. The following day, cells were transfected with 6 μg of VSV-G envelope plasmid, 9 μg of psPAX2 (plasmid encoding viral packaging proteins) and 9 μg of transfer vector plasmid (plasmid encoding the gene of interest) diluted in 1,500 μL Opti- MEM with 70 μL of FuGENE®. Two days after transfection, media was centrifuged at 500 g for 5 minutes to remove cell debris following filtration using 0.45-μm PVDF vacuum filter. The filtered supernatant was concentrated 100-fold using PEG-it Virus Precipitation Solution (System Biosciences; LV825A-1) according to the manufacturer's protocols and resuspended in Opti-MEM serum-free media.

eVLP-Packaged sgRNA Extraction.

RNA was extracted from eVLPs using the QIAmp Viral RNA Mini Kit (Qiagen; 52904) according to the manufacturer's protocols. Extracted RNA was reverse transcribed using SuperScript® III First-Strand Synthesis SuperMix (Thermo Fisher Scientific; 18080400) and an sgRNA-specific DNA primer according to the manufacturer's protocols. The resulting cDNA was used as input for standard high-throughput sequencing preparation to sequence sgRNA barcodes.

Barcoded eVLP Capsid Library Vector Construction.

Oligonucleotide pools containing barcode/capsid variant pairs were synthesized by Twist Biosciences. Oligonucleotide pools were amplified using KAPA HiFi HotStart ReadyMix (Roche Diagnostics) supplemented with 3% (v/v) DMSO. Primers for amplification were added to a final concentration of 0.5 μM. 1 ng of oligonucleotide pool template was added per 25 μL reaction. Approximately 70-100 ng of total oligonucleotide pool was minimally amplified to reduce the probability of PCR crossover recombination that scrambles the linkage between barcode sequence and capsid mutant. Oligonucleotide pools were amplified by PCR using the following protocol: 95° C. for 3 minutes; 6 cycles of 98° C. for 20 seconds, 61° C. for 15 seconds, and 72° C. for 1 minute; 72° C. for 1 minute. Amplified oligonucleotide pools were purified and concentrated using the MinElute Reaction Cleanup Kit (Qiagen). Concentrated, amplified oligonucleotide pools were assembled with pre-digested and gel-purified acceptor vector plasmids via Gibson assembly using NEBuilder HiFi DNA Assembly Master Mix (New England Biolabs) according to the manufacturer's protocols. Assembled products were purified using the MinElute Reaction Cleanup Kit (Qiagen). Electrocompetent cells were generated from NEB® Stable (New England Biolabs) chemically competent E. coli by growing single colonies to mid-log phase, collecting cells by centrifugation at 5,000 g for 1 minute at 4° C., washing with cold 10% (v/v) glycerol, and repeating for a total of four washes. Freshly prepared electrocompetent cells were transferred to a chilled 0.1 cm electroporation cuvette (Bio-Rad) and mixed with the purified, assembled library plasmids. Cells were electroporated using a time-constant protocol with t=5 ms at 1.5 kV. Electroporated cells were recovered at 37° C. for 25 minutes with shaking.

Recovered cells were plated onto 500 cm² plates containing LB media+1.5% agar supplemented with 100 μg/mL carbenicillin and incubated for 16 hours at 37° C.

After overnight incubation, colonies were scraped into LB media, and cells were collected by centrifugation. Gibson-assembled library plasmids were purified using a Plasmid Plus Maxi Kit (Qiagen) according to the manufacturer's protocols. The purified plasmids were digested with BsmBI-v2 (New England Biolabs) overnight at 55° C. according to the manufacturer's protocols, and the digested product was subsequently purified by performing two successive gel extractions using the QIAquick Gel Extraction Kit (Qiagen). Purified digests were assembled with the appropriate PCR-amplified inserts using the NEBridge Golden Gate Assembly Kit BsmBI-v2 (New England Biolabs) according to the manufacturer's protocols. Electroporation, plating, and plasmid isolation from transformed colonies was performed as described above. Library quality was assessed using diagnostic digests to confirm uniform plasmid size, Sanger sequencing of 16-32 colonies to verify the correct barcode/mutant linkage, and high-throughput sequencing of the barcodes to ensure adequate coverage of all library members.

Barcoded eVLP Capsid Library Production Screens.

Lentiviral libraries were produced as described above. In parallel, Gesicle cells were seeded at a density of 3.4×10⁶ cells per T75 flask. Twenty-four hours after seeding, each flask of Gesicle cells was infected with 500 μL of concentrated lentivirus (from 10 mL of viral producer cell supernatant). Twenty-four hours after transduction, the media was changed, and puromycin selection was initiated at a final puromycin concentration of 1 μg/mL. Cell viability was monitored, and the media was exchanged or cells were expanded appropriately. The initial multiplicity of infection (MOI) was inferred by counting surviving cells at 1 week post transduction and assuming a doubling time of 24 hours. In all cases, MOIs between 0.01 and 0.1 were achieved.

After sufficient expansion of the integrated producer cell library, these cells were seeded for eVLP production in T75 flasks at a density of 5×10⁶ cells/flask. At this stage, 5×10⁵ cells were collected by centrifugation, lysed in lysis buffer as described above, and reserved for sequencing analysis of producer-cell integrated barcode sequences. Twenty-four hours after seeding, cells were transfected using the jetPRIME® transfection reagent (Polyplus) according to the manufacturer's protocols. A mixture of pUC19 (5,500 ng), VSV-G (400 ng), and MMLVgag-pro-pol (3,375 ng) plasmids were co-transfected per T-75 flask. Forty to forty-eight hours post transfection, eVLPs were harvested and filtered as described above. The filtered supernatant was concentrated 1000-fold by ultracentrifugation using a cushion of 20% (w/v) sucrose in PBS. Ultracentrifugation was performed at 26,000 rpm for 2 hours (4° C.) using an SW28 rotor in an Optima XPN Ultracentrifuge (Beckman Coulter). Following ultracentrifugation, eVLP pellets were resuspended in cold PBS (pH 7.4). RNA was extracted from purified eVLPs as described above, and extracted RNA was reverse transcribed as described above. The resulting cDNA was amplified by PCR using Phusion® HotStart II polymerase using 2 μL of cDNA input per 25 μL reaction and the following conditions: 95° C. for 3 minutes; 16 cycles of 95° C. for 15 seconds, 61° C. for 20 seconds, and 72° C. for 30 seconds; 72° C. for 1 minutes.

The producer cell genomic DNA collected above was purified from crude lysate using an Agencourt DNAdvance kit (Beckman Coulter) according to the manufacturer's protocols. The resulting purified gDNA was amplified by PCR using Phusion® HotStart II polymerase using 500 ng of gDNA input per 25 μL reaction and the following conditions: 95° C. for 3 minutes; 30 cycles of 95° C. for 15 seconds, 61° C. for 20 seconds, and 72° C. for 30 seconds; 72° C. for 1 minutes. Illumina barcodes were installed as described above, and samples were prepared for sequencing as described above. Samples were sequenced on an Illumina MiSeq® instrument (paired-end read, read 1: 150 cycles, read 2: 0 cycles) using an Illumina MiSeq® 150 v3 Kit (Illumina).

Barcoded eVLP Capsid Library Transduction Screens.

Barcoded eVLP capsid libraries were produced and purified as described above. In parallel, HEK293T cells were seeded in 48-well plates at a density of 40,000 cells/well. 18 h after seeding, treated wells were transduced with 20 μL of 1000-fold concentrated, purified eVLP libraries. 6 h post transduction, cells were washed with PBS, and RNA was extracted from cells using the RNeasy® Plus Mini Kit (Qiagen) according to the manufacturer's protocols. Extracted RNA was reverse transcribed and prepared for high-throughput sequencing as described above, with the modification of 23 cycles of PCR1 amplification.

Barcoded eVLP Capsid Library Screen Data Analysis.

Sequencing reads were demultiplexed using the MiSeq® Reporter software (Illumina). Reads were filtered to ensure an average quality Q>30 and to ensure that the reads contained the correct flanking sequences surrounding the 15-bp barcode sequence. The number of reads containing each unique barcode sequence were quantified using a custom Python script. For quantification purposes, to account for sequencing errors, any reads that contained a sequence that was within two mismatches of a particular barcode sequence in the library were marked as containing that particular barcode sequence.

For calculating barcode frequency enrichments in one population relative to another population (e.g., eVLP-packaged sgRNAs vs. producer cell gDNA), barcodes were first filtered to only analyze barcodes that were found in >100 total reads in both populations. Raw read counts were then converted into reads per million (RPM) with a pseudocount of 1 added for each analyzed barcode, and fold change values were calculated using the RPM values.

Capsid Mutant eVLP Potency Assay.

To assess the potency of individual capsid mutants independently but in parallel, eVLPs were produced in 96-well plates. Gesicle cells were seeded in 96-well plates at a density of 20,000 cells per well. After 24 hours, cells were transfected using the jetPRIME® transfection reagent (Polyplus) according to the manufacturer's protocols. A mixture of plasmids expressing VSV-G (4.3 ng), MMLVgag-pro-pol (36.3 ng), gag-ABE8e (12 ng), and an sgRNA (47.3 ng) were co-transfected per well. Edge wells were avoided. Twenty-four hours after transfection, HEK293T cells were seeded for transduction in separate 96-well plates at a density of 16,000 cells per well. Forty-eight hours after transfecting the Gesicle producer cells, the eVLP-containing supernatant was harvested and pipetted directly onto the seeded HEK293T cells without any additional concentration or purification. 10 μL of crude eVLP-containing supernatant was used to transduce each well of HEK293T cells. Forty-eight hours after transduction, genomic DNA was extracted in 60 μL of lysis buffer as described above. Genomic DNA was amplified and prepared for sequencing as described above to assess editing efficiency (eVLP potency).

### Example 2. Directed Evolution of Engineered Virus-Like Particles with Improved Potency

Engineered virus-like particles (eVLPs) are promising vehicles for delivering gene editing agents in vitro and in vivo that combine advantages of viral and non-viral delivery.

While current-generation v4 eVLPs enable efficient gene editing for certain applications, improvements to eVLP potency and related parameters would expand the scope of eVLPs for research and therapeutic applications. In this Example, a system for the laboratory evolution of eVLPs that enables the discovery of improved eVLP variants with desired properties is described. This evolution system uses barcoded guide RNAs loaded within eVLP-packaged cargos to uniquely encode the identity of each eVLP variant in a library, enabling the identification of improved eVLP variants following selections for desired properties. This system was applied to mutate and select eVLP capsids that support improved eVLP production or transduction, and the beneficial mutations were combined to generate v5 BE-eVLPs (base editor-delivering eVLPs), which exhibited a 3.7-fold increase in potency compared to previous state-of-the-art v4 eVLPs. Analyses of v5 eVLPs suggest that these mutations, including a mutation within the RNA-binding nucleocapsid domain, optimized packaging and delivery of desired ribonucleoprotein cargos instead of native viral genomes.

The ability to safely and efficiently deliver macromolecules into cells within the body (in vivo) is a requirement for the safe and effective use of many emerging therapeutic modalities. Current gene editing technologies, for example, hold great promise for treating genetic disorders^{1,2}, but their therapeutic potential is often constrained by the challenge of delivering gene editing agents into the relevant cell types in vivo³⁻⁵. Existing viral delivery technologies such as adeno-associated virus (AAV) have been used to deliver gene editing agents into several tissues in vivo, including in clinical trials^{3,6-12}. The therapeutic utility of AAV delivery, however, can sometimes be reduced by stringent cargo size restrictions¹³, the possibility of unwanted DNA cargo integration into the genomes of transduced cells^{14,15}, and the prolonged expression of gene editing agents in transduced cells, which increases risks of off-target editing^{3,9,16}. Prolonged expression and increased risks of off-target editing also apply to other viral delivery vectors including lentivirus and adenovirus, in addition to increased risk of immunogenicity compared to AAV in some cases^{3,4,17}. Some non-viral delivery methods, including lipid nanoparticle (LNP) delivery, offer reduced off-target editing by transiently delivering editor-encoding mRNA instead of DNA, but using LNP delivery to achieve therapeutic gene editing in tissues other than the liver remains challenging^{3,18,19}. The therapeutic potential of in vivo gene editing could thus be improved by the development of additional delivery strategies that overcome certain challenges of existing methods.

The use of virus-like particles (VLPs) as vehicles for delivering gene editing agents into cells in vitro or in vivo^{3,16,20-32} has been explored. VLPs comprise viral scaffolds that can package and deliver cargo proteins, ribonucleoproteins (RNPs), or mRNA instead of cargo-encoding viral genomes. For gene editing cargo, a major advantage of protein, RNP, or mRNA delivery is transient exposure of target cells to the editing agent instead of persistent expression that can result from viral delivery. Thus, VLP delivery offers the high transduction efficiency and programmable tissue tropism of viral delivery methods with the transient cargo expression and reduced off-target editing of non-viral delivery methods^{3,16} an ideal combination for gene editing applications.

Several VLP-based strategies for delivering gene editing agents into mammalian cells have been previously described, although few enable efficient in vivo genome editing^{3,16,20-32} Engineered virus-like particles (eVLPs) that enable efficient protein delivery and gene editing in cell culture and in the mouse liver and retina were developed¹⁶. In eVLPs, desired cargo proteins are fused to retroviral gag (capsid) proteins, which direct localization of the cargo into viral particles as they form in producer cells. The gag-cargo linker contains a sequence that is engineered to be cleaved by the co-packaged retroviral protease following particle formation, which releases the cargo inside the particles and subsequently into the transduced cells. Additionally, the cell-type specificity of eVLPs is determined by the envelope glycoprotein used to pseudotype the particles. An optimized fourth-generation (v4) eVLP architecture was developed by iteratively engineering eVLPs to improve cargo loading, cargo release, and component stoichiometry that facilitated efficient in vivo editing. v4 eVLP delivery offers equal or greater on-target editing but reduced off-target editing compared to AAV delivery. eVLP delivery can thus serve as a useful modality for the in vivo delivery of gene editing RNPs or other therapeutic proteins.

Additional improvements to the properties of eVLPs could be useful to maximize their therapeutic potential. In particular, increasing the per-particle potency of eVLPs would enable more efficient gene editing with lower eVLP doses, further improving the safety of eVLP delivery and simplifying the production of eVLPs for large-scale studies. A directed evolution approach, in which large libraries of eVLP variants are generated and subjected to selections for desired properties to identify variants that possess those properties, could provide a powerful and general strategy for developing improved eVLPs. Indeed, directed laboratory evolution has been used extensively to develop viral delivery vectors with desired properties, such as increased serum stability or specialized tissue tropism^{3,33-4}. Existing schemes for evolving viral vectors in the laboratory require each unique viral variant to package a viral genome that encodes that particular variant's identity; sequencing the viral genomes that survive following a selection identifies variants that possess the desired properties. Since eVLPs do not package any viral genetic material, however, they cannot be evolved using existing methods. Therefore, unlocking the potential of directed evolution to improve eVLPs requires a strategy to encode an eVLP variant's identity without using viral genetic material.

In this example, a general system for the directed evolution of eVLPs with desired properties is described. In this eVLP evolution system, each eVLP variant packages RNPs loaded with guide RNAs containing a barcode sequence that uniquely identifies that particular eVLP variant. Therefore, the identities of the eVLP variants that survive a selection are determined by sequencing the barcoded guide RNAs that are present post selection. Using this system, a library of eVLP capsid mutants was generated, and selections were performed to identify capsid mutants that support improved eVLP production from producer cells or improved eVLP transduction of target cells. By combining the beneficial capsid mutations, v5 eVLPs were generated, which exhibit increased RNP packaging, improved cargo release, and a 3.7-fold increase in potency compared to the previous-best v4 eVLPs. One mutation in v5 eVLPs abolishes an interaction that is involved in packaging viral genomes in wild-type viruses but is not required in RNP-packaging eVLPs that lack viral genomes, highlighting the benefits of mutating and selecting eVLP capsids to become more optimal for packaging non-native RNP cargos instead of genomes. These results established a platform for evolving next-generation eVLPs with improved properties.

Barcoded Guide RNAs Enable the Identification of eVLP Variants with Desired Properties.

All directed evolution systems require a way to determine the identity of successful variants following a selection for desired properties^{43,44}. When evolving viral vectors, this requirement can be easily met by ensuring that each unique viral variant packages a genome that encodes the identity of that particular variant⁴². For example, when evolving libraries of viral capsid mutants in which each capsid packages its corresponding viral genome, the identity of capsids that survive selection can be determined by sequencing the surviving viral genetic material. This strategy cannot be applied to determine the identity of eVLP variants that survive selection, however, since eVLPs do not package any viral genetic material.

To overcome this challenge, a strategy to encode the identity of each eVLP variant was employed using barcoded single-guide RNAs (sgRNAs) that are loaded inside eVLP-packaged RNP cargos (FIG. 19A). In this scheme, each eVLP production vector expressed both an eVLP variant and a barcoded sgRNA that uniquely identified that eVLP variant (FIG. 19A). These barcoded eVLP production vectors were introduced into producer cells while ensuring that each producer cell only received a single barcoded vector and therefore produced a single eVLP variant/barcoded sgRNA combination. This strategy generated barcoded eVLP libraries in which each unique eVLP variant packaged sgRNAs containing a unique corresponding barcode (FIG. 19A). After subjecting a barcoded eVLP library to a selection for a desired property, the successful eVLP variants were identified by sequencing the surviving sgRNAs and determining which barcodes were enriched in the post-selection population compared to the input population (FIG. 19A). This scheme for evolving barcoded eVLPs in principle can be used to evolve different eVLP components-including capsid, envelope, cargo, and other structural proteins-by placing the evolving component on the same vector as the barcoded sgRNA when constructing the library of eVLP production vectors. Additionally, this scheme is compatible with a wide range of possible selections for desired properties, including improved particle production, particle stability, or transduction of a particular cell type in vitro or in vivo.

It was validated that barcoded sgRNAs are compatible with functional eVLP production. A 15-bp barcode sequence was inserted into the tetraloop of the sgRNA scaffold (FIG. 23A), since previous studies showed that this location and length of insertion does not disrupt sgRNA function^{45,46}. Additionally, a barcode length of 15 bp would allow for 4¹⁵ (approximately 1 billion) possible barcode sequences, enabling a large maximum library size. For all validation experiments, previously developed v4 eVLPs¹⁶ that package a highly active adenine base editor (ABE8e) RNP cargo⁴⁷ were used. Standard v4 eVLPs were produced by co-transfecting four expression plasmids into producer cells (FIG. 23B), encoding the expression of (1) the gag-ABE fusion, (2) the sgRNA that directs on-target base editing in the transduced cells, (3) the Moloney murine leukemia virus (MMLV) gag-pro-pol polyprotein, which contains the required viral protease and other structural components, and (4) the vesicular stomatitis virus G (VSV-G) envelope protein.

v4 eVLPs containing canonical or tetraloop-barcoded sgRNAs were produced, and their potencies were compared by measuring the on-target base editing efficiencies at the BCL11A enhancer locus in eVLP-transduced HEK293T cells. It was observed that barcoded eVLPs exhibited identical potency compared to standard eVLPs (FIG. 23C). Next, because the evolution scheme requires that the barcoded sgRNA and evolving eVLP component are expressed from the same vector, it was confirmed that a single vector containing both an sgRNA expression cassette and a gag-ABE fusion could support efficient eVLP production and cargo delivery (FIG. 23C). These results indicated that barcoded eVLPs can be produced in a manner that preserves standard eVLP properties.

Finally, it was validated that barcoded eVLPs can be used to distinguish between eVLP variants with different functional properties. To do so, a mock selection was performed using two different eVLP cargo constructs: (1) a standard gag-ABE cargo construct used in v4 eVLPs and (2) a non-functional cargo construct containing an ABE but no gag fusion, which almost completely abolishes ABE cargo loading into eVLPs¹⁶. Each of these two cargo constructs was paired with a unique barcoded sgRNA and lentiviral integration was used to generate producer cells expressing either barcode 1 (gag-ABE) or barcode 2 (ABE only) (FIG. 19B). eVLP production was initiated from a 1:1 mixture of these producer cells by transfecting them with plasmids expressing the remaining eVLP components (MMLV gag-pro-pol and VSV-G). Only the barcode 1 (gag-ABE) producer cells and not barcode 2 (ABE producer cells) could produce functional eVLPs containing substantial amounts of ABE RNP cargo. Accordingly, it was observed that barcode 1 was strongly enriched (93% of sequencing reads) compared to barcode 2 (7% of sequencing reads) in eVLP-packaged sgRNAs, even though barcodes 1 and 2 were present and equally represented in the original producer cell mixture (FIG. 19C). These results demonstrated that barcoded sgRNAs can be used to tag different eVLP variants and that barcodes that are enriched following a selection-in this case, a selection for cargo-loaded eVLP production-identify variants with increased fitness. Collectively, these findings validate certain aspects of the barcoded eVLP evolution system and establish a framework for using barcoded sgRNAs to identify eVLP variants with desired properties.

Constructing and Evolving a Barcoded eVLP Capsid Library.

Next, the barcoded eVLP evolution system was used to evolve eVLP capsids with improved properties. The capsid proteins that are used in v4 eVLPs are identical to the capsid proteins used in wild-type MMLV, which have evolved in nature to be optimal for packaging viral genomes^{48,49}. Therefore, wild-type MMLV capsids are likely not optimal for packaging large, non-native protein cargos like ABEs in eVLPs. It was investigated whether remodeling the internal eVLP capsid surface to optimize for ABE RNP cargo packaging instead of viral genome packaging could substantially improve eVLP properties, including potency per particle, number of cargo molecules packaged per particle, overall particle yield or titer, and particle stability.

To evolve eVLP capsids to become more optimal for packaging ABE RNP cargo, a barcoded eVLP capsid library was designed and constructed. This library was designed to contain 3,762 single-residue mutants of the MMLV gag protein capsid (amino acids 215-313 and 413-479) and nucleocapsid (amino acids 480-513) domains in the gag-ABE cargo construct. In total, 198 gag residues were mutated to every possible other amino acid (FIG. 20A).

A library construction strategy that ensured knowledge of the association between barcodes and mutants was implemented to enable decoding of selection outcomes. Each pre-defined barcode-mutant pair was synthesized using commercial high-throughput pooled oligonucleotide synthesis. These pooled sequences were cloned into lentiviral vector backbones using an initial Gibson assembly followed by a Golden Gate assembly that maintained the correct barcode-mutant linkage even while inserting the intervening promoters and non-evolving gag sequences (FIGS. 24B and 24C). Barcodes were chosen such that no two barcode sequences were within four mismatches of each other to minimize the likelihood of incorrect barcode classification due to DNA sequencing errors during barcode retrieval or mutations during eVLP production. The library cloning protocol was extensively optimized to minimize recombination and ensure retention of the correct barcode/mutant linkage (see Methods).

The plasmid library was used to generate a library of barcoded eVLP capsid variants (FIG. 20A). Lentiviral transduction of producer cells at a low multiplicity of infection maximized the fraction of producer cells that each received a single barcode-capsid variant pair. The transduced cells were expanded into a library of barcoded eVLP producer cells (FIG. 20A). High-throughput sequencing analysis of genomic DNA isolated from the expanded producer cell library revealed that 99% of all barcode sequences were detected (FIG. 25). These results demonstrated the successful generation of barcoded eVLP plasmid and producer cell libraries, laying the foundation for eVLP evolution campaigns.

eVLP Capsid Evolution Revealed how Different Mutations Influence eVLP Properties.

The barcoded eVLP capsid library was evolved using two separate selections (FIG. 20B): (1) a selection for improved eVLP production from producer cells and (2) a selection for improved eVLP transduction of human HEK293T cells. To perform a selection for improved eVLP production, eVLP production was initiated from the barcoded producer cell library by transfecting plasmids expressing the remaining eVLP components (MMLV gag-pro-pol and VSV-G). The resulting library of barcoded eVLP capsid variants was purified, the eVLP-packaged sgRNAs were isolated, and the barcodes that were present after this production selection were sequenced. For each barcode sequence in the library, the eVLP production enrichment was calculated by comparing the frequency of that barcode in eVLP-packaged sgRNAs to the frequency of that barcode in the producer-cell gDNA. In this production selection, barcodes that displayed greater enrichment than the canonical eVLP capsid barcode identified candidate capsid mutants that support improved production compared to the canonical capsid (FIG. 26A). Enriched barcodes, for example, can indicate that those capsid mutants package more RNP cargo molecules per particle than the canonical capsid or are produced at a higher titer, either of which could explain why those particular sgRNAs were more abundant in the produced eVLPs relative to producer-cell genomic DNA.

Approximately 8% of all capsid mutants in the library exhibited an average production enrichment higher than that of the canonical eVLP capsid (FIG. 20C and FIG. 26B). Because the complete MMLV capsid comprises a complex assembly of thousands of capsid subunits, it is likely that the majority of capsid mutations disrupt the carefully orchestrated process of capsid assembly, explaining the rarity of mutants enriched beyond that of the canonical eVLP capsid in the eVLP production selection. The enrichment of a subpopulation of capsid mutants in the production selection beyond canonical eVLPs (FIG. 20C and FIG. 26B) suggested that the wild-type MMLV capsid is not optimal for RNP cargo packaging, and that eVLP capsids can be mutated and selected in the laboratory to become more optimal for this desired function.

In addition to improving eVLP production, the eVLP evolution system was also used to improve transduction of eVLPs into target cells (FIG. 20B). HEK293T cells were incubated with the purified barcoded eVLP capsid library for 6 hours, and sgRNAs that were successfully transduced into target cells were isolated. For each barcode sequence in the library, the eVLP transduction enrichment was calculated by comparing the frequency of that barcode in the transduced HEK293T cells to the frequency of that barcode in the eVLP-packaged sgRNAs prior to incubation with HEK293T cells. Barcodes that were enriched to a higher degree than the canonical v4 eVLP barcode identified capsid mutants that support improved transduction relative to the v4 eVLP capsid (FIG. 27A). Enriched barcodes, for example, could reflect capsid mutants that transduce target cells more potently because they are more stable, or enter target cells more efficiently. It was observed that only 0.7% of all capsid mutants in the library exhibited an average transduction enrichment greater than that of the canonical v4 eVLP capsid (FIG. 20C and FIG. 27B). These findings support a model in which capsid mutants are more likely to improve eVLP production or RNP cargo packaging but rarely improve particle stability, cell entry, or other characteristics that influence transduction.

By integrating the results from both the production and transduction selections, a landscape that reveals how each capsid mutant influences these two fundamental properties of eVLPs was generated (FIG. 20C). The vast majority of capsid mutants exhibited worse production and transduction compared to the canonical v4 eVLP capsid. While a handful of mutants showed selection enrichment that suggest improvements in either production or transduction, virtually no mutants exhibited improvements in both properties, suggesting that eVLP production and transduction efficiencies are dictated by distinct and potentially competing mechanisms.

Certain clusters of capsid mutations consistently impact eVLP production and transduction. For example, R440P or R443P improved transduction but negatively impacted production (FIG. 20C). Conversely, L478K, A479K, or T480H improved production but modestly impaired transduction (FIG. 20C). These observations suggest that remodeling the internal charged surfaces of the eVLP capsid can facilitate optimization of eVLP capsids to better package and deliver RNP cargo. Taken together, the results of the eVLP capsid evolution demonstrated the utility of the barcoded eVLP system, revealed new insights into how different capsid mutations influence eVLP properties, and nominated potentially improved capsid mutants for further characterization.

Evolved Capsid Mutations Improved eVLP Potency.

Based on the results of the production and transduction selections, a set of 36 evolved capsid mutants were selected for further characterization (FIG. 20C). These mutants were chosen based on their positive enrichments in both replicates of either the production or transduction selections; mutants that improved one property without substantially impairing the other property were prioritized (FIG. 20C). To perform a high-throughput assessment of the potency of multiple different variants simultaneously, different eVLP variants were produced in different wells of 96-well plates, HEK293T cells were transduced with the same volume of each eVLP variant at a sub-saturating dose (see Methods), and each variant's potency was determined by measuring adenine base editing efficiencies at the sgRNA-specified target BCL11A enhancer locus in the transduced cells.

Each of the 36 evolved capsid mutants was introduced into the v4 gag-ABE construct, and the other canonical components of the v4 eVLP architecture (wild-type MMLV gag-pro-pol, VSV-G, and standard sgRNA) were used to produce the evolved eVLP variants. In this context, it was observed that most of the evolved mutations did not improve eVLP potency compared with v4 eVLPs (FIG. 28A), indicating that incorporating the evolved capsid mutations into the gag-ABE construct alone was not sufficient to improve potency. Since the processed gag protein expressed in the gag-pro-pol construct, along with the processed gag protein expressed in the gag-ABE construct, both contribute to the overall eVLP capsid, however, the evolved capsid mutations were also incorporated into the gag-pro-pol construct used for eVLP production (FIG. 28B). The evolved Q226P mutation was first incorporated into the gag-pro-pol construct (hereafter referred to as gag^{Q226P}-pro-pol), since the Q226P mutation was the most strongly enriched mutation from the production selection that only modestly impaired transduction (FIG. 20C). Next, the potency of the same 36 evolved capsid mutants in the gag-ABE construct was assessed, but now using the evolved gag^{Q226P}-pro-pol instead of the wild-type MMLV gag-pro-pol. In this context, many of the evolved capsid mutants exhibited 2- to 3-fold increases in base editor delivery potency compared to v4 eVLPs (FIG. 21A). These results suggested that the various evolved capsid mutations incorporated into the gag-ABE construct synergized with the evolved gag^{Q226P}-pro-pol construct to yield improved potency.

In light of the discovery that different gag-ABE and gag-pro-pol capsid mutants might synergize, the effects of incorporating different combinations of evolved mutations into the gag-ABE or gag-pro-pol constructs were systematically evaluated. Five evolved gag-ABE mutants that exhibited the highest potency when paired with the gag^{Q226P}-pro-pol: R501I, D502Q, A505W, C507F, and C507V were selected (FIG. 21A). These highest-performing mutations were all located within the nucleocapsid domain at the C-terminus of gag and therefore might directly interact with the packaged RNP cargo. All possible combinations of each C-terminal mutant and Q226P mutant incorporated into either the gag-ABE only, gag-pro-pol only, or both gag-ABE and gag-pro-pol were investigated (FIG. 21B). Four out of the five C-terminal gag-ABE mutants still performed best when paired with the gag^{Q226P}-pro-pol instead of a matched gag-pro-pol containing that same C-terminal mutant (FIG. 21B). The evolved Q226P mutation, which greatly improves eVLP production without strongly impairing transduction, likely compensates for negative effects on transduction that arise from the evolved C-terminal mutations. These findings illuminated the complex interplay between different evolved capsid mutations and underscored the utility of assessing these mutations in several possible eVLP configurations.

Finally, the potency of these five evolved eVLP variants (R501I, D502Q, A505W, C507F, or C507V gag-ABE mutants paired with gag^{Q226P}-pro-pol) compared to v4 eVLPs across a range of doses in HEK293T cells was evaluated (FIG. 21C). All evolved eVLPs exhibited improved potency at all doses tested compared to v4 eVLPs (FIG. 21C). In particular, the gag^{C507V}-ABE+gag^{Q226P}-pro-pol combination exhibited an average overall 3.7-fold improvement in potency (EC₅₀), achieving the same base editing efficiencies at 3- to 4-fold lower doses compared to v4 eVLPs (FIG. 21C). This substantial improvement in potency is comparable to what was observed between v2 and v1 eVLPs or v3 and v2 eVLPs in a previous study¹⁶. Therefore, the gag^{C507V}-ABE/gag^{Q226P}-pro-pol combination was designated as v5 BE-eVLPs (FIG. 21C). Collectively, these results demonstrated that the barcoded eVLP evolution system successfully evolved eVLP capsid mutants with improved properties, enabling the generation of v5 eVLPs that exhibit 3.7-fold improved potency compared to previous-best v4 eVLPs.

v5 eVLPs Exhibited Improved Cargo Packaging and Release Compared to v4 eVLPs.

To further illuminate the effects of the evolved mutations in v5 eVLPs, their location within gag was analyzed. The evolved Q226P mutation is located at the N-terminus of the capsid domain of gag, directly upstream of the internal protease cleavage site that separates the capsid and p12 domains following particle maturation (FIG. 22A). Due to its proximity to this protease cleavage site, it is possible that the evolved Q226P mutation alters the rate of cleavage at this site, which could impact the kinetics of capsid formation to become more optimal for packaging large RNP cargos. In contrast, the evolved C507V mutation is located near the C-terminus of the nucleocapsid domain of gag (FIG. 22B). The evolved C507V mutation disrupted the second cysteine in the CCHC zinc finger motif within the nucleocapsid domain (FIG. 22B) that is known to be required for packaging and replicating viral genomes in wild-type MMLV⁵⁰⁻⁵². Because eVLPs lack viral genomes, this CCHC zinc finger motif is likely no longer required in eVLPs and is instead free to be mutated during selection for improved RNP cargo packaging. Thus, the barcoded eVLP evolution system identified a capsid mutation that removes a native viral function that is dispensable in eVLPs, further highlighting the benefits of evolving eVLP capsids to become more optimal for packaging non-native RNP cargos instead of genomes.

To experimentally characterize the effects of the evolved mutations, the protein and sgRNA content of v4 and v5 eVLPs was analyzed. In a previous study¹⁶, efficient cargo release was identified as a determinant of eVLP potency. To assess the efficiency of cargo release in v4 and v5 eVLPs, Western blots of lysed eVLPs were performed to determine the fraction of cleaved ABE cargo present after particle maturation (FIG. 22C and FIG. 29). These results revealed more efficient cleavage of the capsid-cargo linker in v5 eVLPs compared to v4 eVLPs (FIG. 22C and FIG. 29), indicating that improved cargo release in v5 eVLPs likely contributes to their improved potency. Next, the number of ABE protein molecules packaged per eVLP was quantified by ELISA. A 1.8-fold increase in protein packaging in v5 eVLPs compared to v4 eVLPs was observed (FIG. 22D). A 4.3-fold increase in the sgRNA packaging levels was also detected by RT-qPCR in v5 eVLPs compared to v4 eVLPs (FIG. 22E). The combined increases in protein and sgRNA packaging suggested that v5 eVLPs packaged substantially more active RNPs per particle compared to v4 eVLPs, which likely contributes to their improved potency.

Previous attempts to improve cargo packaging beyond that of v4 eVLPs resulted in increased protein packaging but not sgRNA packaging¹⁶, and so it is noteworthy that the v5 capsid mutations evolved to improve RNP packaging and not just protein packaging, likely because barcoded sgRNA abundance was used as the readout for all selections and thus directly rewarded improved sgRNA packaging levels. It was observed that v5 eVLPs are equally compatible with barcoded sgRNAs and standard sgRNAs, indicating that the selection outcomes were not strongly influenced by the use of barcoded sgRNAs (FIG. 30). Taken together, these results indicated that v5 eVLPs exhibited improved RNP cargo packaging and release compared to v4 eVLPs, suggesting a mechanistic basis for why the evolved capsid mutations support improved eVLP potency.

To further characterize the physical properties of v5 eVLPs, dynamic light scattering (DLS) and cryo-electron microscopy (cryoEM) analyses were performed. DLS analysis revealed that v5 eVLPs exhibited a similar distribution in particle sizes compared to v4 eVLPs, but v5 eVLPs were slightly larger in diameter on average (172.6±4.5 nm) compared to v4 eVLPs (156.5±4.2 nm) (FIG. 31). Collectively, these analyses illuminated the differences between v5 and v4 eVLPs that may contribute to the improved potency of v5 eVLPs.

Overall, this Example describes a system for the directed evolution of eVLPs with desired properties and application of this system to evolve eVLP capsid mutants with improved potency. The eVLP evolution system, which leveraged barcoded sgRNAs to identify eVLP variants that enrich during selections for desired properties, provided a powerful general approach for developing improved eVLPs. By mutating and selecting eVLP capsids toward enhanced eVLP production and transduction, new v5 BE-eVLPs were developed that exhibit improved RNP cargo packaging, improved cargo release, and a 3.7-fold increase in potency relative to the previous-best v4 eVLPs.

The results of eVLP capsid evolution emphasized the strengths of using directed evolution to improve the properties of eVLPs. Because eVLPs comprise multi-protein assemblies in which each component plays multiple structural and functional roles, it can be challenging to use rational protein engineering to endow eVLPs with specific properties. The approach described in the present Example, which used unbiased capsid mutagenesis followed by selections for improved production and transduction, yielded evolved capsid mutations that would have been very difficult to discover via rational engineering. While VLPs derived from different viruses have been previously described^{3,16,23,24,29}, the v5 eVLPs reported here are the first VLPs that do not use wild-type viral capsids and instead use capsids that were mutated and selected in the laboratory to package the desired RNP cargo. Indeed, using evolved capsids proved to be highly beneficial, since the evolved mutations led to improved RNP cargo packaging by remodeling native capsid:viral genome interactions that are now dispensable in genome-free eVLPs. The capsid mutation and selection campaign also revealed new insights into the properties of eVLPs, illuminating a possible tradeoff between mutations that enhance production versus transduction as well as the complex interplay between mutations incorporated into the gag-ABE versus gag-pro-pol constructs.

In addition to advances in eVLP delivery, these results established a technical framework for constructing barcoded eVLP libraries and performing barcoded eVLP selections. The requirement that each producer cell expresses a single combination of barcode and eVLP component variant facilitates maintaining the prescribed barcode-variant linkage during eVLP production. In this Example, this requirement was achieved using lentiviral transduction at a low multiplicity of infection, but recombination between lentiviral genomes during virus production might disrupt barcode-variant linkage,^{53,54} decreasing the signal-to-noise ratio in selections.

### Methods

### Cloning.

Plasmids used were cloned using USER cloning as described previously¹⁶. DNA was amplified via PCR using Phusion® U Green Multiplex PCR Master Mix (Thermo Fisher Scientific). Machi (Thermo Fisher Scientific) or NEB® Stable (New England Biolabs) chemically competent E. coli were used for plasmid propagation.

### Cell Culture.

HEK293T cells (ATCC; CRL-3216) and Gesicle Producer 293T cells (Takara; 632617) were maintained in DMEM+GlutaMAX (Life Technologies) supplemented with 10% (v/v) fetal bovine serum (Gibco). Cells were cultured at 37° C. with 5% carbon dioxide and were confirmed to be negative for mycoplasma by testing with MycoAlert (Lonza Biologics).

eVLP Production and Purification.

eVLPs were produced as described previously¹⁶. Briefly, eVLPs were produced by transient transfection of Gesicle Producer 293T cells. For medium- to large-scale preparations, Gesicle cells were seeded in T-75 flasks (Corning) at a density of 5×10⁶ cells per flask. After 20-24 hours, cells were transfected using the jetPRIME® transfection reagent (Polyplus) according to the manufacturer's protocols. A mixture of plasmids expressing VSV-G (400 ng), MMLV gag-pro-pol (3,375 ng), gag-ABE (1,125 ng), and an sgRNA (4,400 ng) were co-transfected per T-75 flask.

Forty to forty-eight hours post-transfection, producer cell supernatant was harvested and centrifuged for 5 minutes at 500 g to remove cell debris. The clarified eVLP-containing supernatant was filtered through a 0.45-μm PVDF filter. The filtered supernatant was concentrated 100-fold using PEG-it Virus Precipitation Solution (System Biosciences; LV825A-1) according to the manufacturer's protocols and resuspended in Opti-MEM serum-free media.

eVLP Transduction and Genomic DNA Isolation.

Cells were transduced with eVLPs as described previously¹⁶. Cells were plated for transduction in 48-well plates (Corning) at a density of 30,000-40,000 cells per well. After 20-24 hours, eVLPs were added directly to the culture media in each well. Forty-eight to seventy-two hours post-transduction, cellular genomic DNA was isolated as previously reported¹⁶. Briefly, cells were washed once with PBS and lysed in 150 μL of lysis buffer (10 mM Tris-HCl pH 8.0, 0.05% SDS, 25 μg mL⁻¹ Proteinase K (Thermo Fisher Scientific)) at 37° C. for 1 hour followed by heat inactivation at 80° C. for 30 minutes.

### High-Throughput Sequencing of Genomic DNA.

Genomic DNA was isolated as described above. Following genomic DNA isolation, 1 μL of the isolated DNA (1-10 ng) was used as input for the first of two PCR reactions. Genomic loci were amplified in PCR1 using Phusion® U polymerase (Thermo Fisher Scientific). PCR1 was performed as follows: 95° C. for 3 minutes; 30 cycles of 95° C. for 15 seconds, 61° C. for 20 seconds, and 72° C. for 30 seconds; 72° C. for 1 minute. PCR1 products were confirmed on a 1% agarose gel. 1 μL of PCR1 was used as an input for PCR2 to install Illumina barcodes. PCR2 was conducted for nine cycles of amplification using Phusion® HotStart II polymerase (Thermo Fisher Scientific). Following PCR2, samples were pooled and gel purified in a 1% agarose gel using a QIAquick Gel Extraction Kit (Qiagen). Library concentration was quantified using the Qubit High-Sensitivity Assay Kit (Thermo Fisher Scientific). Samples were sequenced on an Illumina MiSeq® instrument (paired-end read, read 1: 200-280 cycles, read 2: 0 cycles) using an Illumina MiSeq® 300 v2 Kit (Illumina).

### High-Throughput Sequencing Data Analysis for Base Editing Experiments.

Sequencing reads were demultiplexed using the MiSeq® Reporter software (Illumina) and were analyzed using CRISPResso2⁶² as previously described¹⁶. Batch analysis mode (one batch for each unique amplicon and sgRNA combination analyzed) was used in all cases. Reads were filtered by minimum average quality score (Q>30) prior to analysis. The following quantification window parameters were used: -w 20 -wc -10. Base editing efficiencies were reported as the percentage of sequencing reads containing a given base conversion at a specific position. Prism 10 (GraphPad) was used to generate dot plots and bar plots.

### Lentiviral Vector Production.

HEK293T/17 (ATCC CRL-11268) cells were maintained in antibiotic-free DMEM supplemented with 10% fetal bovine serum (v/v). On day 1, 5×10⁶ cells were plated in 10 mL of media in T-75 flasks. The following day, cells were transfected with 6 μg of VSV-G envelope plasmid, 9 μg of psPAX2 (plasmid encoding viral packaging proteins), and 9 μg of transfer vector plasmid (plasmid encoding the gene of interest) diluted in 1,500 μL Opti-MEM with 70 μL of FuGENE® HD transfection reagent (Promega). Two days after transfection, media was centrifuged at 500 g for 5 minutes to remove cell debris following filtration using 0.45-μm PVDF vacuum filter. The filtered supernatant was concentrated using PEG-it Virus Precipitation Solution (System Biosciences; LV825A-1) according to the manufacturer's protocols and resuspended in Opti-MEM serum-free media.

eVLP-Packaged sgRNA Extraction.

RNA was extracted from eVLPs as described previously¹⁶. Briefly, the QIAmp Viral RNA Mini Kit (Qiagen; 52904) was used according to the manufacturer's protocols. Extracted RNA was reverse transcribed using SuperScript™ III First-Strand Synthesis SuperMix (Thermo Fisher Scientific; 18080400) and an sgRNA-specific DNA primer according to the manufacturer's protocols. The resulting cDNA was used as input for standard high-throughput sequencing preparation described above to sequence sgRNA barcodes.

Barcoded eVLP Capsid Library Vector Construction.

Oligonucleotide pools containing barcode/capsid variant pairs were synthesized by Twist Biosciences. Oligonucleotide pools were amplified using KAPA HiFi HotStart ReadyMix (Roche Diagnostics) supplemented with 3% (v/v) DMSO. Primers for amplification were added to a final concentration of 0.5 μM. 1 ng of oligonucleotide pool template was added per 25 μL reaction. ~70-100 ng of total oligonucleotide pool was minimally amplified to reduce the probability of PCR crossover recombination that could scramble the linkage between barcode sequence and capsid mutant. Oligonucleotide pools were amplified by PCR using the following protocol: 95° C. for 3 minutes; 6 cycles of 98° C. for 20 seconds, 61° C. for 15 seconds, and 72° C. for 1 minute; 72° C. for 1 minute. Amplified oligonucleotide pools were purified and concentrated using the MinElute Reaction Cleanup Kit (Qiagen). Concentrated, amplified oligonucleotide pools were assembled with pre-digested and gel-purified acceptor vector plasmids via Gibson assembly using NEBuilder HiFi DNA Assembly Master Mix (New England Biolabs) according to the manufacturer's protocols. Assembled products were purified using the MinElute Reaction Cleanup Kit (Qiagen). Electrocompetent cells were generated from NEB® Stable (New England Biolabs) chemically competent E. coli by growing single colonies to mid-log phase, collecting cells by centrifugation at 5,000 g for 1 minute at 4° C., washing with cold 10% (v/v) glycerol, and repeating for a total of four washes. Freshly prepared electrocompetent cells were transferred to a chilled 0.1 cm electroporation cuvette (Bio-Rad) and mixed with the purified, assembled library plasmids. Cells were electroporated using a time-constant protocol with t=5 ms at 1.5 kV. Electroporated cells were recovered at 37° C. for 25 minutes with shaking. Recovered cells were plated onto 500 cm² plates containing LB media+1.5% agar supplemented with 100 μg/mL carbenicillin and incubated for 16 hours at 37° C.

After overnight incubation, colonies were scraped into LB media, and cells were collected by centrifugation. Gibson-assembled library plasmids were purified using a Plasmid Plus Maxi Kit (Qiagen) according to the manufacturer's protocols. The purified plasmids were digested with BsmBI-v2 (New England Biolabs) overnight at 55° C. according to the manufacturer's protocols, and the digested product was subsequently purified by performing two successive gel extractions using the QIAquick Gel Extraction Kit (Qiagen). Purified digests were assembled with the appropriate PCR-amplified inserts using the NEBridge Golden Gate Assembly Kit BsmBI-v2 (New England Biolabs) according to the manufacturer's protocols. Electroporation, plating, and plasmid isolation from transformed colonies was performed as described above. Library quality was assessed using diagnostic digests to confirm uniform plasmid size, Sanger sequencing of 16-32 colonies to verify the correct barcode/mutant linkage, and high-throughput sequencing of the barcodes to ensure adequate coverage of all library members. Library cloning was performed separately to generate four distinct sub-libraries in which each sub-library contained every capsid mutant within a 150 bp region.

Barcoded eVLP Capsid Library Evolution.

Lentiviral libraries were produced as described above. In parallel, Gesicle cells were seeded at a density of 3.4×10⁶ cells per T-75 flask. Twenty-four hours after seeding, each flask of Gesicle cells was infected with 500 μL of concentrated lentivirus (from 10 mL of viral producer cell supernatant). Twenty-four hours after transduction, the media was changed, and puromycin selection was initiated at a final puromycin concentration of 1 μg/mL. Cell viability was monitored, and cells were expanded upon reaching confluency. The initial multiplicity of infection (MOI) was inferred by counting surviving cells at 1 week post transduction and assuming a doubling time of 24 hours. In all cases, MOIs between 0.01 and 0.1 were achieved.

For production selections, after sufficient expansion of the integrated producer cell library, these cells were seeded for eVLP production in T-75 flasks at a density of 5×10⁶ cells/flask. At this stage, 5×10⁵ cells were collected by centrifugation, lysed in lysis buffer as described above, and reserved for sequencing analysis of producer-cell integrated barcode sequences. Twenty-four hours after seeding, cells were transfected using the jetPRIME® transfection reagent (Polyplus) according to the manufacturer's protocols. A mixture of pUC19 (5,500 ng), VSV-G (400 ng), and MMLV gag-pro-pol (3,375 ng) plasmids were co-transfected per T-75 flask. Forty to forty-eight hours post transfection, eVLPs were harvested and filtered as described above. The filtered supernatant was concentrated 1000-fold by ultracentrifugation using a cushion of 20% (w/v) sucrose in PBS. Ultracentrifugation was performed at 26,000 rpm for 2 hours (4° C.) using an SW28 rotor in an Optima XPN Ultracentrifuge (Beckman Coulter). Following ultracentrifugation, eVLP pellets were resuspended in cold PBS (Gibco, pH 7.4). RNA was extracted from purified eVLPs as described above, and extracted RNA was reverse transcribed as described above. The resulting cDNA was amplified by PCR using Phusion® HotStart II polymerase using 2 μL of cDNA input per 25 μL reaction and the following conditions: 95° C. for 3 minutes; 16 cycles of 95° C. for 15 seconds, 61° C. for 20 seconds, and 72° C. for 30 seconds; 72° C. for 1 minute.

The producer cell genomic DNA collected above was purified from crude lysate using an Agencourt DNAdvance kit (Beckman Coulter) according to the manufacturer's protocols. The resulting purified gDNA was amplified by PCR using Phusion® HotStart II polymerase using 500 ng of gDNA input per 25 μL reaction and the following conditions: 95° C. for 3 minutes; 30 cycles of 95° C. for 15 seconds, 61° C. for 20 seconds, and 72° C. for 30 seconds; 72° C. for 1 minute. Illumina barcodes were installed as described above, and samples were prepared for sequencing as described above. Samples were sequenced on an Illumina MiSeq® instrument (paired-end read, read 1: 150 cycles, read 2: 0 cycles) using an Illumina MiSeq® 150 v3 Kit (Illumina).

For transduction selections, barcoded eVLP capsid libraries were produced and purified as described above. In parallel, HEK293T cells were seeded in 48-well plates at a density of 40,000 cells/well. Eighteen hours after seeding, treated wells were transduced with 20 μL of 1000-fold concentrated, purified eVLP libraries. Six hours post transduction, cells were washed with PBS, and RNA was extracted from cells using the RNeasy® Plus Mini Kit (Qiagen) according to the manufacturer's protocols. Extracted RNA was reverse transcribed and prepared for high-throughput sequencing as described above, with the modification of 23 cycles of PCR1 amplification.

Barcoded eVLP Capsid Library Evolution Enrichment Analysis.

Sequencing reads were demultiplexed using the MiSeq® Reporter software (Illumina). Reads were filtered using fastp63 to ensure an average quality Q>30 and using seqkit⁶⁴ to ensure that the reads contained the correct flanking sequences surrounding the 15-bp barcode sequence. The numbers of reads containing each unique barcode sequence were quantified using a custom Python script. For quantification purposes, to account for sequencing errors, any reads that contained a sequence that was within two mismatches of a particular barcode sequence in the library were marked as containing that particular barcode sequence. For calculating barcode frequency enrichments in one population relative to another population (e.g., eVLP-packaged sgRNAs vs. producer cell gDNA), raw read counts were converted into reads per million (RPM) with a pseudocount of 1 added for each barcode, and fold change values were calculated using the RPM values. Only barcodes that were found in >100 total reads in both pre-selection and post-selection populations were analyzed.

### High-Throughput Capsid Mutant Potency Assay.

To assess the potency of individual capsid mutants independently in a high-throughput fashion, eVLPs were produced in 96-well plates. Gesicle cells were seeded in 96-well plates at a density of 20,000 cells per well. After 24 hours, cells were transfected using the jetPRIME® transfection reagent (Polyplus) according to the manufacturer's protocols. A mixture of plasmids expressing VSV-G (4.3 ng), evolved or wild-type MMLVgag-pro-pol (36.3 ng), evolved gag-ABE8e (12 ng), and an sgRNA targeting the BCL11A enhancer site (47.3 ng) were co-transfected per well. Edge wells were avoided. Twenty-four hours after transfection, HEK293T cells were seeded for transduction in separate 96-well plates at a density of 16,000 cells per well. Forty-eight hours after transfecting the Gesicle producer cells, the eVLP-containing supernatant was harvested and pipetted directly onto the seeded HEK293T cells without any additional concentration or purification. 10 μL of crude eVLP-containing supernatant was used to transduce each well of HEK293T cells. Forty-eight hours after transduction, genomic DNA was extracted in 60 μL of lysis buffer as described above. Genomic DNA was amplified and prepared for sequencing as described above to assess editing efficiency. The fold change in potency relative to v4 eVLPs was calculated by dividing the editing efficiency of the capsid variant by the editing efficiency of v4 eVLPs in the same experiment.

Western Blot Analysis of eVLP Protein Content.

Western blots to analyze the percent of cleaved ABE cargo in v4 versus v5 eVLPs were performed as described previously¹⁶. eVLPs were lysed in Laemmli sample buffer (50 mM Tris-HCl pH 7.0, 2% sodium dodecyl sulfate (SDS), 10% (v/v) glycerol, 2 mM dithiothreitol (DTT)) by heating at 95° C. for 15 minutes. Protein extracts were separated by electrophoresis at 150 V for 45 minutes on a NuPAGE 3-8% Tris-Acetate gel (Thermo Fisher Scientific) in NuPAGE Tris-Acetate SDS running buffer (Thermo Fisher Scientific). Transfer to a PVDF membrane was performed using an iBlot 2 Gel Transfer Device (Thermo Fisher Scientific) at 20 V for 7 minutes. The membrane was blocked for 1 hour at room temperature with rocking in blocking buffer: 1% bovine serum albumin (BSA) in TBST (150 mM NaCl, 0.5% Tween-20, and 50 mM Tris-HCl). After blocking, the membrane was incubated overnight at 4° C. with rocking with mouse anti-Cas9 (Cell Signaling Technology; 14697, 1:1000 dilution). The membrane was washed three times with 1×TBST for 10 minutes each time at room temperature, then incubated with goat anti-mouse antibody (LI-COR IRDye 680RD; 926-68070, 1:10000 dilution in blocking buffer) for 1 hour at room temperature with rocking. The membrane was washed as before and imaged using an Odyssey Imaging System (LI-COR). The relative amounts of cleaved ABE and full-length gag-ABE were quantified by densitometry using ImageJ, and the percent of cleaved ABE relative to total (cleaved+full-length) ABE was calculated.

eVLP Protein Content Quantification.

eVLP protein content quantification was performed as described previously¹⁶. Briefly, eVLPs were lysed in Laemmli sample buffer as described above. The concentration of ABE protein in ultracentrifuge-purified v4 or v5 eVLPs was quantified using the FastScan™ Cas9 (S. pyogenes) ELISA kit (Cell Signaling Technology; 29666C) according to the manufacturer's protocols. Recombinant Cas9 (S. pyogenes) nuclease protein (New England Biolabs; M0386) was used to generate the standard curve for quantification. The concentration of MLV p30 protein in purified eVLPs was quantified using the MuLV Core Antigen ELISA kit (Cell Biolabs; VPK-156) according to the manufacturer's protocols. The number of ABE protein molecules per eVLP was calculated by determining the ratio of Cas9 molecules to p30 molecules and assuming a copy number of 1800 molecules of p30 per eVLP as previously described¹⁶.

eVLP sgRNA Abundance Quantification.

RNA was extracted from eVLPs and reverse transcribed as described above. qPCR analysis of the resulting cDNA was performed using a CFX96 Touch Real-Time PCR Detection System (Bio-Rad) with SYBR green dye (Lonza; 50512). The amount of cDNA input was normalized to MLV p30 content, and the relative sgRNA abundance per eVLP was calculated as log 2[fold change](ΔC_{q}) relative to v4 eVLPs.

Dynamic Light Scattering (DLS) Analysis of eVLPs.

DLS was performed with a Zetasizer (Malvern Panalytical). 5 μL of v4 or v5 eVLPs purified by ultracentrifugation were diluted in 800 μL of PBS. Polystyrene latex standards of 30 nm, 50 nm, and 100 nm were used to calibrate the instrument. Samples were analyzed in cuvettes.

### Statistical Analysis.

Data are presented as mean and standard error of the mean (s.e.m.). No statistical methods were used to predetermine sample size. Statistical analysis was performed using GraphPad Prism software. Sample sizes are described in the figure legends.

### References for Example 2

1. Anzalone, A. V., Koblan, L. W. & Liu, D. R. Genome editing with CRISPR-Cas nucleases, base editors, transposases and prime editors. Nat Biotechnol 38, 824-844 (2020).
2. Doudna, J. A. The promise and challenge of therapeutic genome editing. Nature 578, 229-236 (2020).
3. Raguram, A., Banskota, S. & Liu, D. R. Therapeutic in vivo delivery of gene editing agents. Cell 185, 2806-2827 (2022).
4. van Haasteren, J., Li, J., Scheideler, O. J., Murthy, N. & Schaffer, D. V. The delivery challenge: fulfilling the promise of therapeutic genome editing. Nat Biotechnol 38, 845-855 (2020).
5. Wei, T. et al. Delivery of Tissue-Targeted Scalpels: Opportunities and Challenges for In Vivo CRISPR/Cas-Based Genome Editing. ACS Nano 14, 9243-9262 (2020).
6. Davis, J. R. et al. Efficient prime editing in mouse brain, liver and heart with dual AAVs. Nat Biotechnol (2023).
7. Davis, J. R. et al. Efficient in vivo base editing via single adeno-associated viruses with size-optimized genomes encoding compact adenine base editors. Nat Biomed Eng 6, 1272-1283 (2022).
8. Levy, J. M. et al. Cytosine and adenine base editing of the brain, liver, retina, heart and skeletal muscle of mice via adeno-associated viruses. Nat Biomed Eng 4, 97-110 (2020).
9. Newby, G. A. & Liu, D. R. In vivo somatic cell base editing and prime editing. Mol Ther 29, 3107-3124 (2021).
10. Wang, D., Zhang, F. & Gao, G. CRISPR-Based Therapeutic Genome Editing: Strategies and In Vivo Delivery by AAV Vectors. Cell 181, 136-150 (2020).
11. Maeder, M. L. et al. Development of a gene-editing approach to restore vision loss in Leber congenital amaurosis type 10. Nat Med 25, 229-233 (2019).
12. Li, T. et al. CRISPR/Cas9 therapeutics: progress and prospects. Signal Transduct Target Ther 8, 36 (2023).
13. Wu, Z., Yang, H. & Colosi, P. Effect of Genome Size on AAV Vector Packaging. Molecular Therapy 18, 80-86 (2010).
14. Chandler, R. J., Sands, M. S. & Venditti, C. P. Recombinant Adeno-Associated Viral Integration and Genotoxicity: Insights from Animal Models. Hum Gene Ther 28, 314-322 (2017).
15. Greig, J. A. et al. Integrated vector genomes may contribute to long-term expression in primate liver after AAV administration. Nat Biotechnol (2023).
16. Banskota, S. et al. Engineered virus-like particles for efficient in vivo delivery of therapeutic proteins. Cell 185, 250-265 e216 (2022).
17. Shirley, J. L., de Jong, Y. P., Terhorst, C. & Herzog, R. W. Immune Responses to Viral Gene Therapy Vectors. Mol Ther 28, 709-722 (2020).
18. Loughrey, D. & Dahlman, J. E. Non-liver mRNA Delivery. Acc Chem Res 55, 13-23 (2022).
19. Paunovska, K., Loughrey, D. & Dahlman, J. E. Drug delivery systems for RNA therapeutics. Nat Rev Genet 23, 265-280 (2022).
20. Campbell, L. A. et al. Gesicle-Mediated Delivery of CRISPR/Cas9 Ribonucleoprotein Complex for Inactivating the HIV Provirus. Mol Ther 27, 151-163 (2019).
21. Choi, J. G. et al. Lentivirus pre-packed with Cas9 protein for safer gene editing. Gene Ther 23, 627-633 (2016).
22. Gee, P. et al. Extracellular nanovesicles for packaging of CRISPR-Cas9 protein and sgRNA to induce therapeutic exon skipping. Nat Commun 11, 1334 (2020).
23. Hamilton, J. R. et al. Targeted delivery of CRISPR-Cas9 and transgenes enables complex immune cell engineering. Cell Rep 35, 109207 (2021).
24. Indikova, I. & Indik, S. Highly efficient ‘hit-and-run’ genome editing with unconcentrated lentivectors carrying Vpr.Prot.Cas9 protein produced from RRE-containing transcripts. Nucleic Acids Res 48, 8178-8187 (2020).
25. Lu, Z. et al. Lentiviral Capsid-Mediated Streptococcus pyogenes Cas9 Ribonucleoprotein Delivery for Efficient and Safe Multiplex Genome Editing. CRISPR J 4, 914-928 (2021).
26. Lyu, P., Javidi-Parsijani, P., Atala, A. & Lu, B. Delivering Cas9/sgRNA ribonucleoprotein (RNP) by lentiviral capsid-based bionanoparticles for efficient ‘hit-and-run’ genome editing. Nucleic Acids Res 47, e99 (2019).
27. Lyu, P. et al. Adenine Base Editor Ribonucleoproteins Delivered by Lentivirus-Like Particles Show High On-Target Base Editing and Undetectable RNA Off-Target Activities. CRISPR J 4, 69-81 (2021).
28. Lyu, P., Wang, L. & Lu, B. Virus-Like Particle Mediated CRISPR/Cas9 Delivery for Efficient and Safe Genome Editing. Life (Basel) 10 (2020).
29. Mangeot, P. E. et al. Genome editing in primary cells and in vivo using viral-derived Nanoblades loaded with Cas9-sgRNA ribonucleoproteins. Nat Commun 10, 45 (2019).
30. Yao, X. et al. Engineered extracellular vesicles as versatile ribonucleoprotein delivery vehicles for efficient and safe CRISPR genome editing. J Extracell Vesicles 10, e12076 (2021).
31. Strebinger, D. et al. Cell type-specific delivery by modular envelope design. Nat Commun 14, 5141 (2023).
32. Haldrup, J. et al. Engineered lentivirus-derived nanoparticles (LVNPs) for delivery of CRISPR/Cas ribonucleoprotein complexes supporting base editing, prime editing and in vivo gene modification. Nucleic Acids Res 51, 10059-10074 (2023).
33. Ogden, P. J., Kelsic, E. D., Sinai, S. & Church, G. M. Comprehensive AAV capsid fitness landscape reveals a viral gene and enables machine-guided design. Science 366, 1139-1143 (2019).
34. Byrne, L. C. et al. In vivo-directed evolution of adeno-associated virus in the primate retina. JCI Insight 5 (2020).
35. Dalkara, D. et al. In vivo-directed evolution of a new adeno-associated virus for therapeutic outer retinal gene delivery from the vitreous. Sci Transl Med 5, 189ra176 (2013).
36. Deverman, B. E. et al. Cre-dependent selection yields AAV variants for widespread gene transfer to the adult brain. Nat Biotechnol 34, 204-209 (2016).
37. Maheshri, N., Koerber, J. T., Kaspar, B. K. & Schaffer, D. V. Directed evolution of adeno-associated virus yields enhanced gene delivery vectors. Nat Biotechnol 24, 198-204 (2006).
38. Tabebordbar, M. et al. Directed evolution of a family of AAV capsid variants enabling potent muscle-directed gene delivery across species. Cell 184, 4919-4938 e4922 (2021).
39. Hwang, B. Y. & Schaffer, D. V. Engineering a serum-resistant and thermostable vesicular stomatitis virus G glycoprotein for pseudotyping retroviral and lentiviral vectors. Gene Ther 20, 807-815 (2013).
40. Vu, H. N., Ramsey, J. D. & Pack, D. W. Engineering of a stable retroviral gene delivery vector by directed evolution. Mol Ther 16, 308-314 (2008).
41. Chan, K. Y. et al. Engineered AAVs for efficient noninvasive gene delivery to the central and peripheral nervous systems. Nat Neurosci 20, 1172-1179 (2017).
42. Bartel, M. A., Weinstein, J. R. & Schaffer, D. V. Directed evolution of novel adeno-associated viruses for therapeutic gene delivery. Gene Ther 19, 694-700 (2012).
43. Wang, Y. et al. Directed Evolution: Methodologies and Applications. Chem Rev 121, 12384-12444 (2021).
44. Packer, M. S. & Liu, D. R. Methods for the directed evolution of proteins. Nat Rev Genet 16, 379-394 (2015).
45. Shechner, D. M., Hacisuleyman, E., Younger, S. T. & Rinn, J. L. Multiplexable, locus-specific targeting of long RNAs with CRISPR-Display. Nat Methods 12, 664-670 (2015).
46. Zhu, S. et al. Guide RNAs with embedded barcodes boost CRISPR-pooled screens. Genome Biol 20, 20 (2019).
47. Richter, M. F. et al. Phage-assisted evolution of an adenine base editor with improved Cas domain compatibility and activity. Nat Biotechnol 38, 883-891 (2020).
48. Qu, K. et al. Structure and architecture of immature and mature murine leukemia virus capsids. Proc Natl Acad Sci USA 115, E11751-E11760 (2018).
49. Basyuk, E., Boulon, S., Skou Pedersen, F., Bertrand, E. & Vestergaard Rasmussen, S. The packaging signal of MLV is an integrated module that mediates intracellular transport of genomic RNAs. J Mol Biol 354, 330-339 (2005).
50. Gorelick, R. J. et al. Characterization of the block in replication of nucleocapsid protein zinc finger mutants from moloney murine leukemia virus. J Virol 73, 8185-8195 (1999).
51. D'Souza, V. & Summers, M. F. Structural basis for packaging the dimeric genome of Moloney murine leukaemia virus. Nature 431, 586-590 (2004).
52. Yu, Q. & Darlix, J. L. The zinc finger of nucleocapsid protein of Friend murine leukemia virus is critical for proviral DNA synthesis in vivo. J Virol 70, 5791-5798 (1996).
53. Adamson, B., Norman, T. M., Jost, M. & Weissman, J. S. Approaches to maximize sgRNA-barcode coupling in Perturb-seq screens. bioRxiv (2018).
54. Feldman, D., Singh, A., Garrity, A. J. & Blainey, P. C. Lentiviral co-packaging mitigates the effects of intermolecular recombination and multiple integrations in pooled genetic screens. bioRxiv (2018).
55. Xu, C. et al. piggyBac mediates efficient in vivo CRISPR library screening for tumorigenesis in mice. Proc Natl Acad Sci USA 114, 722-727 (2017).
56. Anzalone, A. V. et al. Programmable deletion, replacement, integration and inversion of large DNA sequences with twin prime editing. Nat Biotechnol 40, 731-740 (2022).
57. Hamilton, J. R. et al. Programmable enveloped delivery vehicles for human genome engineering in vivo. bioRxiv (2023).
58. Dobson, C. S. et al. Antigen identification and high-throughput interaction mapping by reprogramming viral entry. Nat Methods 19, 449-460 (2022).
59. Yu, B. et al. Engineered cell entry links receptor biology with single-cell genomics. Cell 185, 4904-4920 e4922 (2022).
60. Dahlman, J. E. et al. Barcoded nanoparticles for high throughput in vivo discovery of targeted therapeutics. Proc Natl Acad Sci USA 114, 2060-2065 (2017).
61. Sago, C. D. et al. High-throughput in vivo screen of functional mRNA delivery identifies nanoparticles for endothelial cell gene editing. Proc Natl Acad Sci USA 115, E9944-E9952 (2018).
62. Clement, K. et al. CRISPResso2 provides accurate and rapid genome editing sequence analysis. Nat Biotechnol 37, 224-226 (2019).
63. Chen, S., Zhou, Y., Chen, Y. & Gu, J. fastp: an ultra-fast all-in-one FASTQ preprocessor. Bioinformatics 34, i884-i890 (2018).
64. Shen, W., Le, S., Li, Y. & Hu, F. SeqKit: A Cross-Platform and Ultrafast Toolkit for FASTA/Q File Manipulation. PLoS One 11, e0163962 (2016).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method for generating a library of cells capable of producing virus-like particles (VLPs) comprising:
transfecting a plurality of polynucleotides encoding VLPs into producer cells,
wherein each polynucleotide encodes a variant of at least one component of the VLP or its cargo,
wherein the variant comprises at least one amino acid substitution relative to a VLP that does not comprise a variant of at least one component of the VLP or its cargo; and
wherein each polynucleotide encoding a VLP component or cargo variant is associated with a unique nucleic acid barcode sequence.

2. (canceled)

3. A method for evolving virus-like particles (VLPs) comprising:
(i) transfecting a plurality of polynucleotides encoding VLPs into producer cells,
wherein each polynucleotide encodes a variant of at least one component of the VLP or its cargo, wherein each variant comprises at least one amino acid substitution relative to a VLP that does not comprise a variant of at least one component of the VLP or its cargo; and
wherein each polynucleotide encoding a VLP component or cargo variant is associated with a unique nucleic acid barcode sequence;
(ii) producing VLPs from the producer cells; and
(iii) selecting VLPs having at least one improved property by determining the abundance of each barcode sequence relative to the abundance of a barcode sequence associated with a VLP that does not comprise a variant of at least one component of the VLP or its cargo;
wherein VLPs comprising variants associated with barcode sequences that are present at higher abundance relative to the barcode sequence associated with the VLP that does not comprise a variant of at least one component of the VLP or its cargo have at least one improved property.

4-64. (canceled)

65. A group specific antigen (gag) protein comprising a viral nucleocapsid protein variant that comprises an amino acid sequence at least 80% identical to the amino acid sequence of SEQ ID NO: 100, wherein the amino acid sequence of the viral nucleocapsid protein variant comprises one or more substitutions at positions selected from the group consisting of 215, 216, 218, 219, 220, 221, 222, 223, 224, 225, 226, 227, 228, 229, 230, 231, 232, 233, 234, 235, 236, 237, 238, 239, 240, 241, 242, 244, 245, 246, 249, 250, 253, 254, 255, 256, 257, 258, 259, 260, 261, 262, 263, 264, 265, 266, 269, 271, 272, 274, 275, 276, 277, 279, 280, 281, 282, 283, 285, 286, 288, 289, 291, 292, 293, 294, 295, 296, 297, 298, 299, 300, 301, 302, 303, 304, 305, 307, 308, 310, 311, 418, 420, 421, 424, 427, 430, 432, 433, 435, 436, 437, 438, 440, 441, 443, 444, 446, 447, 448, 449, 452, 455, 458, 460, 463, 464, 466, 467, 468, 469, 470, 471, 472, 473, 474, 475, 477, 478, 479, 480, 481, 482, 483, 485, 486, 487, 488, 489, 490, 491, 492, 495, 496, 497, 498, 499, 500, 501, 502, 503, 504, 505, 506, 507, 508, 509, 510, 511, and 512 relative to the amino acid sequence provided in SEQ ID NO: 100, or at corresponding positions in a homologous viral nucleocapsid protein.

66. The gag protein of claim 65, wherein the amino acid sequence of the viral nucleocapsid protein comprises one or more substitutions selected from the group consisting of F215L, F215G, F215M, P216K, P2161, R2181, R218G, R218H, A219C, A219K, A219N, A219G, G220C, G221W, G221M, N222H, N2221, N222M, N222Y, N222V, G223K, G223A, G223C, G223D, Q224L, Q224R, Q224F, Q224I, L225Q, Q226P, Y227S, W228N, W228F, P229D, P229L, F230C, F230E, S231Y, S23iF, S232L, S232A, S233K, S233I, S233R, D234E, D234A, L235C, L235M, Y236E, Y236M, N237D, W238D, K239A, K239T, K239H, N240A, N240E, N240L, N240S, N240I, N241K, N241V, N242T, S244Y, S244M, S244T, F245H, F245R, F245W, S246L, S246H, S246F, S246V, S246Y, P249S, P249F, P249K, G250C, G250D, G250L, G250R, T253F, A254W, L255H, L255V, I256V, I256W, E257A, E257C, S258W, S258V, V259R, L260M, L260W, L2601, 1261K, I261W, I261Q, T262N, T262W, T262F, T262Q, H263I, Q264S, Q264T, P265G, P265F, T266C, D269G, Q27iF, Q271A, Q271D, Q272P, Q272G, L274W, G275W, T276R, T276V, T276M, L277W, T279G, G280W, G280D, G280Y, E281T, E281N, E281C, E282S, E282H, E282Y, K283F, R285V, V286H, L288D, L288K, L288A, E289C, R291K, K292L, A293H, A293Y, V294Q, R295M, G296D, D297N, D297A, D297M, D297P, D297W, D298Y, G299M, G299R, G299Y, R300L, P301S, P301L, T302V, Q303S, L304N, P305R, P305G, P305M, E307N, V308R, V308I, A310R, A311F, A311E, A311M, K418Y, L420C, G421D, G421A, G421R, V424Y, A427G, I430W, I430H, N432Y, K433P, E435A, T436N, P437K, E438H, R440C, R440P, E441M, R443P, I444E, R446G, R446I, E447H, E447S, T448M, T448W, T448V, E449D, E452S, R455Q, E458F, E460W, E463H, K464L, R466Q, R466A, D467E, R468Q, R468W, R468I, R468S, R469Q, R470A, R470M, R470W, H471M, H471D, H471G, H47iN, R472E, R472D, E473N, M474W, M474C, M474I, S475G, L477W, L477C, L477E, L478K, L478V, L478P, A479Q, A479L, A479K, A479S, A479Y, T480H, T480W, T480E, V48iT, V482L, V482M, S483W, S483L, S483R, Q485K, Q485G, Q485L, Q485W, K486L, K486I, K486V, K486E, K486F, Q487P, Q487E, D488Q, D488I, R489A, R489K, R489M, R489F, Q490T, G491C, G491Q, G491H, G492Q, G492W, G492H, R495Y, R495K, R495F, R495I, R496L, R496N, R496F, R496A, R496K, S497C, S497P, S497Q, S497T, S497N, Q498K, Q498V, L499Y, L499F, L499G, L499T, D500Q, D500G, D500M, D500I, R501D, R501I, R501L, D502Q, D502A, D502P, Q503T, C504N, C504S, A505M, A505Y, A505W, A505I, Y506L, Y506M, C507F, C507V, C507G, C507W, K508D, K508N, E509G, E509A, E509M, K510M, K510P, K510R, G511L, G511K, G511P, H512Q, H512S, and H512M relative to the amino acid sequence provided in SEQ ID NO: 100, or corresponding substitutions in a homologous viral nucleocapsid protein.

67. The gag protein of claim 65, wherein the amino acid sequence of the viral nucleocapsid protein variant comprises one or more substitutions at positions selected from the group consisting of 222, 226, 227, 228, 229, 230, 235, 239, 240, 244, 245, 246, 253, 254, 256, 260, 261, 272, 276, 277, 279, 292, 293, 297, 305, 308, 418, 427, 440, 443, 463, 466, 467, 470, 471, 472, 473, 477, 478, 479, 482, 483, 485, 496, 497, 499, 500, 505, 506, and 507 relative to the amino acid sequence provided in SEQ ID NO: 100, or at corresponding positions in a homologous viral nucleocapsid protein.

68. The gag protein of claim 67, wherein the amino acid sequence of the viral nucleocapsid protein comprises one or more substitutions selected from the group consisting of N222H, N222I, Q226P, Y227S, W228N, P229D, F230C, L235C, K239A, N240A, S244Y, F245H, F245R, S246L, S246H, T253F, A254W, I256V, L260M, I261K, Q272P, T276R, L277W, T279G, K292L, A293H, D297N, D297A, P305R, V308R, K418Y, A427G, R440P, R443P, E463H, R466Q, D467E, R470A, H471M, R472E, E473N, L477W, L478K, A479Q, A479L, V482M, S483W, Q485K, R496L, S497C, L499Y, L499F, D500Q, A505M, Y506L, C507F relative to the amino acid sequence provided in SEQ ID NO: 100, or corresponding substitutions in a homologous viral nucleocapsid protein.

69. The gag protein of claim 67, wherein when incorporated into a VLP the gag protein facilitates higher packaging of one or more cargo molecules in the VLP and/or increased production of the VLP in a producer cell.

70-71. (canceled)

72. The gag protein of claim 65, wherein the amino acid sequence of the viral nucleocapsid protein variant comprises one or more substitutions at positions selected from the group consisting of 215, 219, 226, 22, 261, 272, 280, 283, 288, 293, 310, 46, 469, 471, 472, 478, 479, 480, 482, 485, 490, 492, 496, 497, 500, 501, 502, 504, 505, 507, and 508 relative to the amino acid sequence provided in SEQ ID NO: 100, or at corresponding positions in a homologous viral nucleocapsid protein.

73. The gag protein of claim 72, wherein the amino acid sequence of the viral nucleocapsid protein comprises one or more substitutions selected from the group consisting of F215G, A219C, Q226P, E257C, I261W, Q272G, G280W, K283F, L288A, A293Y, A310R, D467E, R469Q, H471D, H471M, R472E, L478K, A479K, T480H, V482M, Q485L, 0485K, Q490T, G492W, R496F, S497Q, D500Q, R501D, R5011, D502Q, C504N, A505M, A505W, C507F, C507V, and K508D relative to the amino acid sequence provided in SEQ ID NO: 100, or corresponding substitutions in a homologous viral nucleocapsid protein.

74. The gag protein of claim 72, wherein when incorporated into a VLP the gag protein facilitates improved transduction efficiency of the VLP into a target cell type.

75-80. (canceled)

81. The gag protein of claim 65, wherein the gag protein comprises an amino acid substitution at position 226 relative to SEQ ID NO: 100, or at a corresponding position in a homologous viral nucleocapsid protein.

82. (canceled)

83. The gag protein of claim 65, wherein the gag protein comprises an amino acid substitution at position 501 relative to SEQ ID NO: 100, or at a corresponding position in a homologous viral nucleocapsid protein.

84-85. (canceled)

86. The gag protein of claim 65, wherein the gag protein comprises an amino acid substitution at position 507 relative to SEQ ID NO: 100, or at a corresponding position in a homologous viral nucleocapsid protein.

87. (canceled)

88. The gag protein of claim 65, wherein the gag protein comprises amino acid substitutions at positions 226 and 501 relative to SEQ ID NO: 100, or at corresponding positions in a homologous viral nucleocapsid protein.

89. The gag protein of claim 88, wherein the amino acid substitutions are Q226P and R501D, or Q226P and R501I.

90-94. (canceled)

95. A VLP comprising a gag protein of claim 65.

96-127. (canceled)

128. A polynucleotide encoding the gag protein of claim 65.

129. A vector comprising the polynucleotide of claim 128.

130-131. (canceled)

132. A pharmaceutical composition comprising the VLP of claim 95.

133. (canceled)

134. A method comprising transfecting or transducing a target cell with the VLP of claim 95.

135-139. (canceled)


SEQUENCE LISTING (112 sequences)

SEQ ID NO: 1 - protein, 10 aa, Synthetic construct
TSTLLMENSS

SEQ ID NO: 2 - protein, 11 aa, Synthetic construct
PRSSLYPALTP

SEQ ID NO: 3 - protein, 8 aa, Synthetic construct
VQALVLTQ

SEQ ID NO: 4 - protein, 12 aa, Synthetic construct
PLQVLTLNIERR

SEQ ID NO: 5 - protein, 8 aa, Synthetic construct
ASPRAGGK

SEQ ID NO: 6 - protein, 8 aa, Synthetic construct
YEADSLEE

SEQ ID NO: 7 - protein, 6 aa, Synthetic construct
YQYRAL

SEQ ID NO: 8 - protein, 6 aa, Synthetic construct
LGVLIV

SEQ ID NO: 9 - protein, 18 aa, Synthetic construct
EGRGSLLTCGDVEENPGP

SEQ ID NO: 10 - protein, 19 aa, Synthetic construct
ATNFSLLKQAGDVEENPGP

SEQ ID NO: 11 - protein, 20 aa, Synthetic construct
QCTNYALLKLAGDVESNPGP

SEQ ID NO: 12 - protein, 22 aa, Synthetic construct
VKQTLNFDLLKLAGDVESNPGP

SEQ ID NO: 13 - protein, 7 aa, Synthetic construct
PKKKRKV

SEQ ID NO: 14 - protein, 30 aa, Synthetic construct
MDSLLMNRRKFLYQFKNVRWAKGRRETYLC

SEQ ID NO: 15 - protein, 9 aa, Synthetic construct
PAAKRVKLD

SEQ ID NO: 16 - protein, 18 aa, Synthetic construct
KRTADGSEFESPKKKRKV

SEQ ID NO: 17 - protein, 17 aa, Synthetic construct
KRTADGSEFEPKKKRKV

SEQ ID NO: 18 - protein, 19 aa, Synthetic construct
NLSKRPAAIKKAGQAKKKK

SEQ ID NO: 19 - protein, 11 aa, Synthetic construct
RQRRNELKRSF

SEQ ID NO: 20 - protein, 38 aa, Synthetic construct
NQSSNFGPMKGGNFGGRSSGPYGGGGQYFAKPRNQGGY

SEQ ID NO: 21 - protein, 16 aa, Xenopus sp.
  VARIANT 3..12: note = X may be any naturally occurring amino acid
KRXXXXXXXXXXKKKL

SEQ ID NO: 22 - protein, 14 aa, Synthetic construct
MEELSQALASSFSV

SEQ ID NO: 23 - protein, 12 aa, Synthetic construct
PLQLPPLERLTL

SEQ ID NO: 24 - protein, 12 aa, Synthetic construct
NELALKLAGLDI

SEQ ID NO: 25 - protein, 15 aa, Synthetic construct
ERFEMFRELNEALEL

SEQ ID NO: 26 - protein, 15 aa, Synthetic construct
DHAEKVAEKLEALSV

SEQ ID NO: 27 - protein, 15 aa, Synthetic construct
QLVEELLKIICAFQL

SEQ ID NO: 28 - protein, 15 aa, Synthetic construct
TNLEALQKKLEELEL

SEQ ID NO: 29 - protein, 15 aa, Synthetic construct
DVKEEMTSALATMRV

SEQ ID NO: 30 - protein, 15 aa, Synthetic construct
STNGSLAAEFRHLQL

SEQ ID NO: 31 - protein, 15 aa, Synthetic construct
PSVQELTEQIHRLLM

SEQ ID NO: 32 - protein, 15 aa, Synthetic construct
MNFKELKDFLKELNI

SEQ ID NO: 33 - protein, 15 aa, Synthetic construct
ENFEILMKLKESLEL

SEQ ID NO: 34 - protein, 14 aa, Synthetic construct
FETVYELTKMCTIR

SEQ ID NO: 35 - protein, 15 aa, Synthetic construct
SGKASSSLGLQDFDL

SEQ ID NO: 36 - protein, 15 aa, Synthetic construct
PKYSDIDVDGLCSEL

SEQ ID NO: 37 - protein, 15 aa, Synthetic construct
VDLACTPTDVRDVDI

SEQ ID NO: 38 - protein, 15 aa, Synthetic construct
YGEKTTQRDLTELEI

SEQ ID NO: 39 - protein, 15 aa, Synthetic construct
RRIYDITNVLEGIGL

SEQ ID NO: 40 - protein, 15 aa, Synthetic construct
AKIIPYSGLLLVITV

SEQ ID NO: 41 - protein, 15 aa, Synthetic construct
LRSEEVHWLHVDMGV

SEQ ID NO: 42 - protein, 15 aa, Synthetic construct
LQSEEVHWLHLDMGV

SEQ ID NO: 43 - protein, 15 aa, Synthetic construct
LQVRKYSLDLASLIL

SEQ ID NO: 44 - protein, 15 aa, Synthetic construct
AGVEAIIRILQQLLF

SEQ ID NO: 45 - protein, 15 aa, Synthetic construct
TGVEALIRILQQLLF

SEQ ID NO: 46 - protein, 15 aa, Synthetic construct
IVLNQLCVRFFGLDL

SEQ ID NO: 47 - protein, 15 aa, Synthetic construct
SLGGFEITPPVVLRL

SEQ ID NO: 48 - protein, 15 aa, Synthetic construct
EAIQDLCLAVEEVSL

SEQ ID NO: 49 - protein, 15 aa, Synthetic construct
DELLQVLRMMVGVNI

SEQ ID NO: 50 - protein, 15 aa, Synthetic construct
SVMLAVQEGIDLLTF

SEQ ID NO: 51 - protein, 10 aa, Synthetic construct
LSSHFQELSI

SEQ ID NO: 52 - protein, 15 aa, Synthetic construct
QSTHVDIRTLEDLLM

SEQ ID NO: 53 - protein, 15 aa, Synthetic construct
ESSAEDLRTLQQLFL

SEQ ID NO: 54 - protein, 15 aa, Synthetic construct
EFSLPTHHTVRLIRV

SEQ ID NO: 55 - protein, 15 aa, Synthetic construct
MSSGYYLGEILRLAL

SEQ ID NO: 56 - protein, 15 aa, Synthetic construct
DTVLDILRDFFELRL

SEQ ID NO: 57 - protein, 15 aa, Synthetic construct
NSVNEILSEFYYVRL

SEQ ID NO: 58 - protein, 15 aa, Synthetic construct
CAFLSVKKQFEELTL

SEQ ID NO: 59 - protein, 15 aa, Synthetic construct
ISPEHVIQALESLGF

SEQ ID NO: 60 - protein, 15 aa, Synthetic construct
AHWMRQLVSFQKLKL

SEQ ID NO: 61 - protein, 15 aa, Synthetic construct
ATRELDELMASLSDF

SEQ ID NO: 62 - protein, 15 aa, Synthetic construct
YQNIELITFINALKL

SEQ ID NO: 63 - protein, 15 aa, Synthetic construct
FNATAVVRHMRKLQL

SEQ ID NO: 64 - protein, 15 aa, Synthetic construct
SGIFGLVTNLEELEV

SEQ ID NO: 65 - protein, 15 aa, Synthetic construct
EESYTLNSDLARLGV

SEQ ID NO: 66 - protein, 15 aa, Synthetic construct
EESYDLTSHLARLGV

SEQ ID NO: 67 - protein, 15 aa, Synthetic construct
GIQQAHAEQLANMRI

SEQ ID NO: 68 - protein, 15 aa, Synthetic construct
AAEPVILDLRDLFQL

SEQ ID NO: 69 - protein, 10 aa, Synthetic construct
MEGCVSNLMV

SEQ ID NO: 70 - protein, 9 aa, Synthetic construct
EGCVSNLMV

SEQ ID NO: 71 - protein, 15 aa, Synthetic construct
DMDFLRNLFSQTLSL

SEQ ID NO: 72 - protein, 15 aa, Synthetic construct
EQLLEIVHDLENLSL

SEQ ID NO: 73 - protein, 15 aa, Synthetic construct
NVMKYFTDLFDYLPL

SEQ ID NO: 74 - protein, 15 aa, Synthetic construct
KVYPIILRLGSNLSL

SEQ ID NO: 75 - protein, 15 aa, Synthetic construct
YAGFSLPHAILRIDL

SEQ ID NO: 76 - protein, 15 aa, Synthetic construct
EIVRDIKEKLCYVAL

SEQ ID NO: 77 - protein, 15 aa, Synthetic construct
EAINKLESNLRELQI

SEQ ID NO: 78 - protein, 15 aa, Synthetic construct
EAINKLENNLRELQI

SEQ ID NO: 79 - protein, 15 aa, Synthetic construct
SDQKQEQLLLKKMYL

SEQ ID NO: 80 - protein, 15 aa, Synthetic construct
KQVLWDRTFSLFQQL

SEQ ID NO: 81 - protein, 15 aa, Synthetic construct
AQLQNLTKRIDSLPL

SEQ ID NO: 82 - protein, 15 aa, Synthetic construct
NDENEHQLSLRTVSL

SEQ ID NO: 83 - protein, 15 aa, Synthetic construct
ISFTEFVKVLEKVDV

SEQ ID NO: 84 - protein, 14 aa, Synthetic construct
MESAITLWQFLLQL

SEQ ID NO: 85 - protein, 15 aa, Synthetic construct
VPKELMQQIENFEKI

SEQ ID NO: 86 - protein, 15 aa, Synthetic construct
QARFILEKIDGKIII

SEQ ID NO: 87 - protein, 15 aa, Synthetic construct
QVKFIKMIIEKELTV

SEQ ID NO: 88 - protein, 15 aa, Synthetic construct
NHRMKNLREISQLGI

SEQ ID NO: 89 - protein, 15 aa, Synthetic construct
NHRVKKLNEISKLGI

SEQ ID NO: 90 - protein, 15 aa, Synthetic construct
TEKHLQKYLRQDLRL

SEQ ID NO: 91 - protein, 15 aa, Synthetic construct
RQERKRPLLDLHIEL

SEQ ID NO: 92 - protein, 15 aa, Synthetic construct
ANMRIQDLKVSLKPL

SEQ ID NO: 93 - protein, 15 aa, Synthetic construct
ATMRVDYEQIKIKKI

SEQ ID NO: 94 - protein, 15 aa, Synthetic construct
LQGEEFVCLKSIILL

SEQ ID NO: 95 - protein, 15 aa, Synthetic construct
THYGQKAILFLPLPV

SEQ ID NO: 96 - protein, 15 aa, Synthetic construct
PSAHEITGLADSLQL

SEQ ID NO: 97 - protein, 15 aa, Synthetic construct
VRLHDVLHSDKKLTL

SEQ ID NO: 98 - protein, 15 aa, Synthetic construct
LINRNGELKLANFGL

SEQ ID NO: 99 - protein, 15 aa, Synthetic construct
LEPLKKLECLKSLDL

SEQ ID NO: 100 - protein, 538 aa, unidentified
  source 1..538: note = Moloney murine leukemia virus
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDD

SEQ ID NO: 101 - DNA, 37 bp, Synthetic construct
  source 1..37: mol_type = other DNA
acactctttccctacacgacgctcttccgatctnnnn

SEQ ID NO: 102 - DNA, 179 bp, Synthetic construct
  source 1..179: mol_type = other DNA
tcttggctttatatatcttgtggaaaggacgaaacaccgtttatcacaggctccaggaagtttaagagctatgctggaagatccatcgacatcaacagcatagcaagtttaaataaggctagtccgttatcaacttgaaaaagtggcaccgagtcggtgctttttttgagacattgatt

SEQ ID NO: 103 - DNA, 179 bp, Synthetic construct
  source 1..179: mol_type = other DNA
aatcaatgtctcaaaaaaagcaccgactcggtgccactttttcaagttgataacggactagccttatttaaacttgctatgctgttgatgtcgatggatcttccagcatagctcttaaacttcctggagcctgtgataaacggtgtttcgtcctttccacaagatatataaagccaaga

SEQ ID NO: 104 - DNA, 29 bp, Synthetic construct
  source 1..29: mol_type = other DNA
tggagttcagacgtgtgctcttccgatct

SEQ ID NO: 105 - protein, 538 aa, Synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLPYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDD

SEQ ID NO: 106 - protein, 538 aa, Synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDDDQCAYCKEKGHWAKDCPKKPRGPRGPRPQTSLLTLDD

SEQ ID NO: 107 - protein, 538 aa, Synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDIDQCAYCKEKGHWAKDCPKKPRGPRGPRPQTSLLTLDD

SEQ ID NO: 108 - protein, 538 aa, Synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYVKEKGHWAKDCPKKPRGPRGPRPQTSLLTLDD

SEQ ID NO: 109 - protein, 538 aa, Synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLPYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDDDQCAYCKEKGHWAKDCPKKPRGPRGPRPQTSLLTLDD

SEQ ID NO: 110 - protein, 538 aa, Synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLPYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDIDQCAYCKEKGHWAKDCPKKPRGPRGPRPQTSLLTLDD

SEQ ID NO: 111 - protein, 31 aa, Synthetic construct
PVADSTTSQAFPLRAGGNGQLPYWPFSSSDL

SEQ ID NO: 112 - protein, 31 aa, Synthetic construct
RRRSQLDRDQCAYVKEKGHWAKDCPKKPRGP